(19) United States
(12) Patent Application Publication
(10) Pub. No.: US 2015/0250733 A1
(43) Pub. Date: Sep. 10, 2015

(54) ORAL DRUG DELIVERY FORMULATIONS

(71) Applicant: Isa ODIDI, Toronto (CA)

(72) Inventor: Isa ODIDI, Toronto (CA)

(21) Appl. No.: 14/685,214

(22) Filed: Apr. 13, 2015

Related U.S. Application Data
(63) Continuation of application No. PCT/CA2013/000610, filed on Jun. 28, 2013.
(60) Provisional application No. 61/714,182, filed on Oct. 15, 2012.

(51) Int. Cl. A61K 9/28; A61K 31/485; A61K 9/20; A61K 31/437; A61K 31/197; A61K 31/137; A61K 31/138; A61K 31/167

(52) U.S. Cl. CPC A61K 9/2846; A61K 31/138; A61K 31/485; A61K 31/167; A61K 31/437; A61K 31/197; A61K 31/137; A61K 9/2086

(57) ABSTRACT

In an aspect, a formulation is provided that comprises at least one active substance and at least one coat comprising Eudragit E (dimethylaminoethyl methacrylate copolymer), wherein the formulation is free of any active substance external to the coat. The formulation is effective in preventing significant dose dumping in alcoholic/non-alcoholic beverage(s). In another aspect, a formulation is provided that comprises a loading dose having at least one active substance, wherein the release of the at least one active substance shows a Point Of Divergence (POD), in a dissolution profile, with the loading dose representing a point in a timeline where the history of the dissolution or release rate changes from an onset of action to another set of points in the timeline represented by a controlled release. The formulation may be used for releasing up to about 55% of a total dose as a loading dose in order to manage pain.

### RELATED APPLICATIONS

This application is a continuation U.S. Application under 35 U.S.C. §365 of International Patent Application No. PCT/CA2013/000610, filed on Jun. 28, 2013, which claims priority to U.S. Provisional Patent Application No. 61/714,182 filed on Oct. 15, 2012, the disclosures of each of which are incorporated by reference herein in their entirety.

### FIELD OF THE INVENTION

The present invention relates to drug delivery formulations, in particular, oral drug delivery formulations, uses thereof and methods of making same.

### BACKGROUND

Modified release, delayed release, controlled release or extended release formulations, in particular solid oral formulations, are often presented as compressed or formed dosage units (as opposed to fine discrete particles or granules) with well-appointed physical geometries which may be expressed internally as matrices and externally as round or shaped tablets, capsules, rods or beads. These geometries help present a physical form and barrier to quick or sudden entry of fluids to the micro environments within these systems when these formulations are brought in contact with a liquid milieu. When compromised, quick and sudden exposure to fluids results in rapid loss of modified release, delayed release, controlled release or extended release properties.

It is well known that with modified release, delayed release, controlled release or extended release formulations, the release of active ingredient depends on certain physicochemical properties of the retarding/gelling agents and or polymeric and non-polymeric agents used to construct the formulations. However, it is also well known that in order for these types of formulations to act in accordance with their properties (such as retard the release of active ingredients in a controlled and or predictable manner), all the Ingredients must be brought into close proximity by compaction. The presentation of these formulations as an Intact matrix (with a solid physical geometry), whether formed or compressed, guarantees this close proximity which in turn provides a conducive environment for the active ingredient release retardation mechanism to kick in effectively.

There has always been the concern that patients may be, inadvertently, administered crushed, modified release, delayed release, controlled release or extended release formulations despite the very clear direction in product literature inserts or prescription labels/instructions not to crush the tablets before use. There have been reports of patients mistakenly sub-dividing or crushing and ingesting these tablets despite instructions not to do so, and suffering serious adverse effects as a result.

A major issue of great concern; however, is that the need for a solid physical geometry for modified release, delayed release, controlled release or extended release formulations to operate effectively is now being exploited by unscrupulous individuals to tamper with or perturb these types of modified release, delayed release, controlled release or extended release formulations in order to rapidly release the active ingredients contained in them. The perturbation or tampering involves one or a combination of crushing, milling, pulverizing, grinding, flattening, heating, microwaving, freezing and cutting to obtain finely divided powders, granules or coarse particles in order to instantaneously obtain the benefit of the total active ingredient present in the slow or delayed release formulation by releasing or dumping all the active ingredient at once in the presence of a liquid milieu. Products containing active ingredients that will produce an emotional, psychological, euphoric, depressive or generally psychedelic experiences are particularly vulnerable.

As an example of this practice, is the well-documented abuse of modified release, delayed release, controlled release or extended release medicinal opioid formulations. This has almost become a way of life to a rapidly growing segment of the world population, especially in the United States and Canada, so much so that, the abuse of opioid products by perturbation or tampering, is now a growing public health concern in the United States and Canada.

Tampering or perturbation of modified release, delayed release, controlled release or extended release solid formulations of opioid analgesic taught in prior art and currently commercialized, occur via heating, microwaving, freezing and/or perturbation or pulverization or crushing or grinding or milling or cutting into one or more sizes ranging from very fine to coarse particles, granules or spheres thereby making it available for instantaneous wetting and thus easy to be abused by the parenteral, nasal or oral route. Another route of abuse is by chewing or licking.

Yet another route of abuse which has become of serious concern is snorting of fine powder obtained from crushed opioid formulation or the oral ingestion of finely crushed extended release oral formulation in order to instantaneously obtain the benefit of the total opioid present in the slow release formulation.

Currently, many formulations and methods currently taught can be compromised and destroyed leading to the loss of controlled release effects and complete release or dose dumping of its opioid content and the rising incidence of opioid abuse. The increase in opioid abuse is particularly evident among young people. In light of this, the Food and Drug Administration (FDA) in the United States has encouraged the development of novel interventions to prevent this abuse, while recognizing the importance of maintaining the availability of these important drug products for the millions of patients who suffer from chronic pain. Numerous stakeholders have also recommended the development of tamper-resistant formulations ever since the first reports of extensive tampering of a commercially available extended release formulation of Oxycodone surfaced.

Unfortunately, to the best of our knowledge, successful new formulations have been elusive or non-existent. Particularly elusive are formulations which do not show complete loss or which show none or insignificant loss in controlled release properties when heated, microwaved, freezed and/or perturbed, pulverized or crushed or ground or milled or cut Into one or more sizes ranging from very fine to coarse particles, granules or spheres. Others that are equally elusive are successful new formulations that do not instantaneously release all or significant amounts of active content when heated, microwaved, freezed and/or pulverized or crushed or ground or milled or cut into one or more sizes ranging from very fine to coarse particles, granules or spheres. Also elusive are successful or effective new formulations which do not release significant amounts or all of its active content over a short period of time when heated, microwaved, freezed and/or pulverized or crushed or ground or milled or cut into one or more sizes ranging from very fine to coarse particles, granules or spheres; formulations with a loading dose having quick onset of action and sustained action that are effective and do not release significant amounts or all of its active content over a short period of time when heated, microwaved, freezed and/or pulverized or crushed or ground or milled or cut into one or more sizes ranging from very fine to coarse particles, granules or spheres; and formulations with a loading dose having quick onset of action and sustained action that are effective and do not dose dump in the presence of alcohol (i.e. alcoholic beverage).

Attempts have been made in the past to control the abuse potential associated with opioid analgesics. Parenteral dose of opioid analgesics are more potent as compared to the same dose administered orally. Therefore, drug abuse is often carried out by the extraction of the opioid from the formulation, and the subsequent injection of the opioid (using any “suitable” vehicle for injection) in order to achieve a “high.” Attempts to curtail abuse have therefore typically centered on the inclusion in the oral formulation of an opioid antagonist which is not orally active but which will substantially block the analgesic effects of the opioid if one attempts to dissolve the opioid and administer it parenterally.

U.S. Pat. No. 3,254,088 is directed to the preparation of naloxone and its activity as a narcotic antagonist. U.S. Pat. No. 3,493,657 is directed to the combination of morphine and naloxone as a composition for parenteral use “which has a strong analgesic, as well as antagonistic effect, without the occurrence of undesired or dangerous side effects.” A New York Times article appearing in a Jul. 14, 1970 issue described the oral administration of naloxone to narcotic addicts as a method of treatment. The oral administration of naloxone (in high doses) “makes it impossible for the addict to experience a high no matter how much heroin he uses.”

The combination of pentazocine and naloxone has been utilized in tablets available in the United States, commercially available as Talwin® from Sanofi-Winthrop. Talwin® contains pentazocine hydrochloride equivalent to 50 mg base and naloxone hydrochloride equivalent to 0.5 mg base. Taiwin® is indicated for the relief of moderate to severe pain. The amount of naloxone present in this combination has no action when taken orally, and will not interfere with the pharmacologic action of pentazocine. However, this amount of naloxone given by injection has profound antagonistic action to narcotic analgesics. Thus, the inclusion of naloxone is intended to curb a form of abuse of oral pentazocine, which occurs when the formulation is solubilized and injected. Therefore, this dosage has lower potential for parenteral abuse than previous oral pentazocine formulations. However, it is still subject to patient misuse and abuse by the oral route, for example, by the patient taking multiple doses at once.

U.S. Pat. No. 6,627,635 is directed to a method of preventing abuse of opioid formulations wherein an analgesically effective amount of an orally active opioid agonist is combined with an opioid antagonist into an oral formulation.

U.S. Pat. No. 6,696,088 is directed to a tamper-resistant oral opioid agonist formulations comprising (i) an opioid agonist in releasable form and (ii) a sequestered opioid antagonist which is substantially not released when the formulation is administered intact, such that the ratio of the amount of antagonist released from said formulation after tampering to the amount of the antagonist released from the intact formulation is about 4:1 or greater, wherein the agonist and antagonist are interdispersed and are not isolated from each other in two distinct layers.

U.S. Pat. No. 7,955,619 is directed to an abuse resistant oral pharmaceutical composition, comprising: a barrier layer, comprising a first polymer; a diffusion layer comprising a second polymer, substantially covering the barrier layer, wherein the diffusion layer is bonded to the barrier layer and comprises a drug that is substantially homogeneously distributed within the second polymer and diffuses from the diffusion layer within the gastrointestinal (GI) tract; and optionally an expansion layer comprising an expandable polymer, wherein the expansion layer is substantially covered by the barrier layer. Methods of making the same and methods of using the same are also provided.

U.S. Pat. No. 3,980,766 is directed to the incorporation of an ingestible solid, which causes a rapid increase in viscosity (gelling) upon concentration of an aqueous solution thereof.

U.S. Pat. No. 4,070,494 is directed to the incorporation of a non-toxic, water gellable material in an amount sufficient to render the drug resistant to aqueous extraction, thus retarding the release of the drug substance.

U.S. Pat. No. 6,309,668 is directed to a tablet for oral administration containing two or more layers comprising one or more drugs and one or more gelling agents within separate layers of the tablet. The examples in this patent all describe conventional immediate release formulations and the resulting tablets form a gel when combined with the volume of water necessary to dissolve the drug; this formulation thus reduces the extractability of the drug from the tablet.

Although these compositions may preclude abuse by injections, this approach would fail to prevent abuse by crushing and swallowing or snorting the formulation. It should also be noted that reduction of extractability was only reported in small volumes of water.

U.S. Pat. Nos. 6,277,384, 6,375,957 and 6,475,494 are directed to oral formulations including a combination of an orally active opioid agonist and an orally active opioid antagonist in a ratio that, when delivered orally, is analgesically effective but that is aversive in a physically dependent subject. While such a formulation may be successful in deterring abuse, it also has the potential to produce adverse effects in legitimate patients.

U.S. Patent Application Publication No. 2007/0066537 is directed to an abuse resistant opioid wherein the opioid is bound to niacin, biotin or peptide. U.S. Patent Application Publication No. 2006/0104909 is directed to a pharmaceutical composition comprising an opioid and a tamper-resistant matrix comprising one or more tenacious cross-linked polymers that are capable of bonding with the opioid such that the opioid is substantially incapable of immediate release from the polymer. U.S. Patent Application Publication No. 2005/0281748 is directed to an opioid bound to a lipid or fatty acid to produce an abuse resistant drug. U.S. Pat. No. 7,943,173 is directed to a pharmaceutical composition comprising from 10 to 40 mg of oxycodone or a pharmaceutically acceptable salt thereof and 0.65 to 0.90 mg naloxone or a pharmaceutically acceptable salt thereof. U.S. Pat. No. 7,914,818 is directed to oral formulations, comprising (i) a therapeutically effective amount of an opioid agonist; (ii) an opioid antagonist in releasable form; and (iii) a sequestered opioid antagonist which is not released when the formulation is administered intact. U.S. Pat. No. 7,201,920 is directed to therapeutic pharmaceutical compositions comprising a mixture including (a) at least one specific opioid analgesic (b) gel forming polyethylene oxide; (c) at least one specific disintegrant; and (d) a nasal tissue irritant, wherein the composition functions such that less than about 50% of the total amount of opioid analgesic in the composition is recovered when about 490 mg. of the composition is contacted with 15 ml of water. U.S. Pat. No. 7,842,307 is directed to an oral formulation comprising a therapeutically effective amount of an opioid analgesic, an opioid antagonist and one or more pharmaceutically acceptable excipients. U.S. Pat. Nos. 7,842,307 and 7,201,920 do not, however, solve the problem of solubilization and significant drug release seen when crushed.

U.S. Pat. No. 7,674,799 is directed to an oral formulation comprising particles having from about 5 mg to about 320 mg oxycodone hydrochloride active pharmaceutical ingredient. The particles are coated with an amount of hydrophobic material effective to provide a sustained release. U.S. Pat. No. 6,488,963 is directed to a non-film controlled release pharmaceutical formulation comprising an effective amount of a therapeutic compound and a high molecular weight poly(ethylene oxide). U.S. Pat. No. 7,776,314 is directed to a parenteral abuse-proofed solid formulation for oral administration, comprising, in addition to one or more active ingredients with potential for abuse selected from the group consisting of opiates, opioids, tranquillizers, stimulants and narcotics, at least one viscosity-increasing agent. U.S. Pat. No. 8,114,383 is directed to a thermoformed formulation comprising: i) one or more active ingredients with abuse potential (A) selected from the group consisting of opiates and opioids, ii) optionally physiologically acceptable auxiliary substances (B), iii) at least 30% by weight of polyalkylene oxide (C) having a molecular weight of 1-15 million according to rheological measurements, and iv) optionally at least one wax (D), wherein the formulation has a breaking strength of at least 500 N and wherein the active ingredient with abuse potential (A) is present in a controlled release matrix of component (C).

Despite all the above attempts in the prior art to address, the problem of improper administration of medications and their use in a non-indicated or non-prescribed manner resulting in abuse, drug overdose, addiction, suboptimal efficacy or death and the problems of dose dumping in the presence of alcohol and tampering of solid formulations by way of heating, microwaving, freezing and/or perturbation, pulverizing or crushing or grinding or milling or cutting them into one or more sizes ranging from very fine to coarse particles, granules or spheres leading to a faster release of the active content or all of its content, the problem persists. This is partly because of design faults in the formulations or compositions and addicts coming up with creative ways to beat the anti-tampering mechanism or patients and healthcare practitioners not adhering to instructions not to crush or sub-divide such systems. At present, the problem is escalating at an alarming rate with devastating financial, health and social consequences.

Moreover, the aforementioned formulation/composition design shortcomings have resulted in inadequacies in the treatment of severe to moderate pain using opioid analgesics. With current formulations, it may take from 1 to 4 hours before the patient experiences adequate pain relief. The design of abuse/tamper resistant formulations and compositions has compromised timely delivery of onset of pain relief in the quest to impart tamper resistance on opioid formulations and prevent their abuse by way of very hard to crush tablets. In other words, current formulations/compositions have resulted in less effective and, therefore, less optimal treatment of pain in the management of moderate to severe pain when a continuous, around-the-clock opioid analgesic is needed for an extended period of time.

For these reasons, there is still a need for a stable drug delivery system that can be reproducibly manufactured, provide adequate and timely drug release, and yet reduce the potential for abuse/tampering.

### SUMMARY

In accordance with one aspect, there is provided a formulation comprising: at least one primary active substance; and at least one coat comprising Eudragit E (dimethylaminoethyl methacrylate copolymer), wherein the formulation is free of any active substance external to said at least one coat.

In accordance with another aspect, onset of action of said at least one primary active substance is potentiated by the presence of a loading dose comprising said at least one primary active substance. In another aspect, the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release. In another aspect, prior to the POD, there is no significant controlled release. In another aspect, the formulation further comprises at least one secondary active substance, wherein the formulation has a quick onset of action of said at least one primary active substance followed by a controlled release of at least one secondary active substance, or vice versa, wherein said at least one primary active substance and said at least one secondary active substance are the same or different. In another aspect, the formulation further comprises at least one secondary active substance, wherein said at least one primary active substance in the loading dose is released at a higher rate in comparison to another dose having said at least one secondary active substance, wherein said at least one primary active substance and said at least one secondary active substance are the same or different. In another aspect, the formulation further comprises a core, wherein said at least one primary active substance is incorporated into the core, external to the core, or a combination thereof. In another aspect, the formulation further comprises a maintenance dose having at least one secondary active substance, wherein said at least one secondary active substance is the same or different than said at least one primary active substance. In another aspect, the maintenance dose comprises said at least one secondary active substance in a controlled release matrix. In another aspect, the formulation comprises at least one layer of said at least one loading dose and at least one layer of said at least one maintenance dose. In another aspect, said at least one layer of said at least one loading dose covers at least a portion of said at least one layer of said at least one maintenance dose or vice versa, forming a layered formulation. In another aspect, said at least one coat surrounds said layered formulation. In another aspect, the formulation further comprises a core having said at least one maintenance dose and at least one coat comprising said at least one loading dose. In another aspect, the formulation further comprises at least one coat comprising at least one maintenance dose, which said at least one maintenance dose in the core is the same or different than said maintenance dose in said at least one coat. In another aspect, the core further comprises said at least one loading dose and/or said at least one coat of said loading dose further comprises said at least one maintenance dose, which said at least one loading dose in the core is the same or different than said loading dose in said at least one coat. In another aspect, said at least one coat comprising said at least one loading dose significantly covers said core. In another aspect, the formulation further comprises a core, wherein said at least one loading dose and said at least one maintenance dose are external to the core. In another aspect, said at least one coat comprising the Eudragit E further comprises at least one active substance, wherein said at least one active substance and said at least one primary active substance are the same or different. In another aspect, said at least one coat controls the release of said at least one primary active substance. In another aspect, release of any active substance in the formulation is activated by a pH dependent mechanism, ion-exchange dependent mechanism, bacterial flora/enzymes dependent mechanism, or a combination thereof. In another aspect, the pH for the pH dependent mechanism is at most about 5. In another aspect, the ion-exchange mechanism is controlled by at least one ion-exchange resin. In another aspect, said at least one ion-exchange resin is selected from Cholestyramine, Colestipol, Sodium polystyrene sulfonate, Polacrilex resin, and/or Polacrilin potassium. In another aspect, the bacterial flora/enzymes dependent mechanism is controlled by at least one polymer reactive to intestinal bacterial flora/enzymes. In another aspect, said at least polymer is selected from polysaccharides such as guar gum, inulin, chondrotin sulphate, alginates, and/or dextran. In another aspect, the Eudragit E comprises Eudragit E 100™. In another aspect, up to about 55% of the total dose is released as a loading dose to manage pain. In another aspect, the loading dose is released within about 60 minutes of ingestion. In another aspect, the formulation is configured such that when the formulation is administered in a physically compromised form to a subject, the rate of release of said at least one primary active substance in the loading dose is substantially the same or lower than the rate of release of said at least one primary active substance in the loading dose when the formulation is administered in an Intact form. In another aspect, when the formulation is pulverized/milled and added to an alcoholic and/or non-alcoholic beverage, the rate of release of said at least one primary active substance in the loading dose is substantially the same or lower than the rate of release of said at least one primary active substance in the loading dose when the formulation is administered in an intact form. In another aspect, the beverage is an alcoholic beverage. In another aspect, the formulation comprises at least one excipient, wherein dissolution of the pulverized/milled formulation in alcoholic and/or non-alcoholic beverages causes the formulation to agglomerate. In another aspect, the at least one excipient comprises at least one sweliable material in such an amount that dissolution of the pulverized/milled formulation in alcoholic and/or non-alcoholic beverages causes the formulation to agglomerate. In another aspect, said at least one swellable material is at least one pH independent polymer. In another aspect, said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state. In another aspect, said at least one swellable material comprises hydrophobic polymers. In another aspect, the hydrophobic polymers are selected from Eudragit RL, Eudragit NE, Eudragit RS and/or Eudragit NM. In another aspect, the at least one excipient comprises polyethylene oxide and Eudragit RL. In another aspect, the formulation further comprises at least one swellable material in such an amount that dissolution of the pulverized/milled formulation in alcoholic and/or non-alcoholic beverages causes the formulation to agglomerate, the amount of swellable material ranges from about 15 wt % to about 90 wt % of the maintenance dose and/or loading dose. In another aspect, the formulation is objectionable to chewing, sucking, licking and/or holding in the mouth. In another aspect, the formulation further comprises a bittering agent and/or irritant. In another aspect, the formulation is an oral formulation. In another aspect, the formulation is a solid unit form. In another aspect, the surface area covered by the Eudragit E in said at least one coat is greater than 5 mg/cm². In another aspect, the surface area covered by the Eudragit E in the coat is greater than 10 mg/cm². In another aspect, the surface area covered by the Eudragit E in the coat is greater than 20 mg/cm². In another aspect, the surface area covered by the Eudragit E in the coat is from about 5 mg/cm² to about 100 mg/cm². In another aspect, the surface area covered by the Eudragit E in the coat is from about 10 mg/cm² to about 100 mg/cm². In another aspect, the surface area covered by the Eudragit E in the coat is from about 20 mg/cm² to about 100 mg/cn2. In another aspect, the formulation is capable of withstanding about a 350 N force. In another aspect, the formulation is effective in preventing significant dose dumping in any beverage. In another aspect, the formulation further comprises at least one acid to facilitate release of any active substance in the formulation. In another aspect, the formulation further comprises at least one organic acid to facilitate release of any active substance in the formulation, wherein at least one of said at least one loading dose, said at least one maintenance dose, or said at least one coat comprises said at least one organic acid. In another aspect, at least one of said at least one loading dose and said at least one coat comprises said at least one organic acid and the wt % ratio of the organic acid to said at least one primary active substance is from about 1:100 to about 100:1. In another aspect, said at least one loading dose comprises from about 1 wt % to about 15 wt % by weight of said at least one organic acid based on the weight of the loading dose. In another aspect, said at least one maintenance dose comprises from about 1 wt % to about 10 wt % by weight of said at least one organic acid based on the weight of the maintenance dose. In another aspect, said at least one coat comprises from about 5 wt % to about 100 wt % by weight of said at least one organic acid based on the weight of said at least one coat. In another aspect, the formulation further comprises an overcoat, wherein the overcoat comprises said at least one organic acid and at least one polymer. In another aspect, the amount of said at least one organic acid is from about 5 wt % to less than about 100 wt % of the overcoat. In another aspect, said organic acid is selected from lactic acid, phosphoric acid, citric acid, malic acid, fumaric acid, stearic acid, tartaric acid, benzoic acid, or combinations thereof. In another aspect, said at least one primary active substance is an addictive substance. In another aspect, the addictive substance is an opiod agonist and/or a narcotic analgesic.

In accordance with another aspect, there is provided a formulation comprising: a loading dose having at least one primary active substance, wherein the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release.

In another aspect, the onset of action is a quick onset of action. In another aspect, the loading dose represents an active substance that is released at a higher rate in comparison to another dose of an active substance in the same formulation, wherein said at least one primary active substance and said active substance are the same or different. In another aspect, a greater amount of said at least one primary active substance is released in a certain time interval in comparison to another dose of an active substance in the formulation that is released in a similar time interval, wherein said at least one primary active substance and said active substance are the same or different. In another aspect, the formulation further comprises at least one secondary active substance, wherein the formulation has the onset of action of said at least one primary active substance followed by the controlled release of at least one secondary active substance, or vice versa, wherein said at least one primary active substance and said at least one secondary active substance are the same or different. In another aspect, prior to the POD, there is no significant controlled release. In another aspect, the formulation further comprising a core, wherein said at least one primary active substance is incorporated into the core, external to the core, or a combination thereof. In another aspect, the formulation further comprises a maintenance dose having at least one secondary active substance, wherein said at least one secondary active substance is the same or different than said at least one primary active substance. In another aspect, the maintenance dose comprises said at least one secondary active substance in a controlled release matrix. In another aspect, the formulation comprises at least one layer of said at least one loading dose and at least one layer of said at least one maintenance dose. In another aspect, said at least one layer of said at least one loading dose covers at least a portion of said at least one layer of said at least one maintenance dose or vice versa, forming a layered formulation. In another aspect, at least one coat surrounds said layered formulation. In another aspect, the formulation further comprises a core having said at least one maintenance dose and at least one coat comprising said at least one loading dose. In another aspect, the formulation further comprises at least one coat comprising at least one maintenance dose, which said at least one maintenance dose in the core is the same or different than said maintenance dose in said at least one coat. In another aspect, the core further comprises said at least one loading dose and/or said at least one coat of said loading dose further comprises said at least one maintenance dose, which said at least one loading dose in the core is the same or different than said loading dose in said at least one coat. In another aspect, said at least one coat comprising said at least one loading dose significantly covers said core. In another aspect, the formulation further comprises a core, wherein said at least one loading dose and said at least one maintenance dose are external to the core. In another aspect, the release of any active substance is activated by a pH dependent mechanism, ion-exchange dependent mechanism, bacterial flora/enzymes dependent mechanism, or a combination thereof. In another aspect, the pH for the pH dependent mechanism is at most about 5. In another aspect, the ion-exchange mechanism is controlled by at least one ion-exchange resin. In another aspect, said at least one ion-exchange resin is selected from Cholestyramine, Colestipol, Sodium polystyrene sulfonate, Polacrilex resin, and/or Polacrilin potassium. In another aspect, the bacterial flora/enzymes dependent mechanism is controlled by at least one polymer reactive to intestinal bacterial flora/enzymes. In another aspect, said at least polymer is selected from polysaccharides such as guar gum, inulin, chondrotin sulphate, alginates, and/or dextran. In another aspect, the formulation comprises at least one excipient, wherein dissolution of the pulverized/milled formulation in alcoholic and/or non-alcoholic beverages causes the formulation to agglomerate. In another aspect, the at least one excipient comprises at least one swellable material in such an amount that dissolution of the pulverized/milled formulation in alcoholic and/or non-alcoholic beverages causes the formulation to agglomerate. In another aspect, said at least one swellable material is at least one pH independent polymer. In another aspect, said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state. In another aspect, said at least one swellable material comprises hydrophobic polymers. In another aspect, the hydrophobic polymers are selected from Eudragit RL, Eudragit NE, Eudragit RS and/or Eudragit NM. In another aspect, the at least one excipient comprises polyethylene oxide and Eudragit RL. In another aspect, the formulation further comprises at least one sweliable material in such an amount that dissolution of the pulverized/milled formulation in alcoholic and/or non-alcoholic beverages causes the formulation to agglomerate, the amount of swellable material ranges from about 15 wt % to about 90 wt % of the maintenance dose and/or loading dose. In another aspect, the formulation further comprises at least one acid to facilitate release of any active substance in the formulation. In another aspect, the formulation further comprises at least one organic acid to facilitate release of any active substance in the formulation, wherein at least one of said at least one loading dose or said at least one maintenance dose comprises said at least one organic acid. In another aspect, said at least one loading dose comprises from about 1 wt % to about 15 wt % by weight of said at least one organic acid based on the weight of the loading dose. In another aspect, said at least one maintenance dose comprises from about 1 wt % to about 10 wt % by weight of said at least one organic acid based on the weight of the maintenance dose. In another aspect, said at least one loading dose comprises said at least one organic acid. In another aspect, the formulation further comprises an overcoat, wherein the overcoat comprises said at least one organic acid and at least one polymer. In another aspect, the amount of said at least one organic acid is from about 5 wt % to less than about 100 wt % of the overcoat. In another aspect, said organic acid is selected from lactic acid, phosphoric acid, citric acid, malic acid, fumaric acid, stearic acid, tartaric acid, benzoic acid, or combinations thereof. In another aspect, said at least one primary active substance is an addictive substance. In another aspect, the addictive substance is an opiod agonist and/or a narcotic analgesic. In another aspect, up to about 55% of the total dose is released as a loading dose to manage pain. In another aspect, the loading dose is released within about 60 minutes of ingestion.

In accordance with yet another aspect, there is provided a formulation comprising: at least one primary active substance; and at least one excipient, wherein dissolution of the intact and or pulverized/milled formulation in alcoholic and/or non-alcoholic beverages causes the formulation to agglomerate.

In another aspect, the at least one excipient comprises at least one swellable material in such an amount that dissolution of the pulverized/milled formulation in alcoholic and/or non-alcoholic beverages causes the formulation to agglomerate. In another aspect, said at least one swellable material is at least one pH independent polymer. In another aspect, said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state. In another aspect, said at least one swellable material comprises hydrophobic polymers. In another aspect, the hydrophobic polymers are selected from Eudragit RL, Eudragit NE, Eudragit RS and/or Eudragit NM. In another aspect, the at least one excipient comprises polyethylene oxide and Eudragit RL. In another aspect, the formulation further comprises at least one organic acid to facilitate release of any active substance in the formulation. In another aspect, the formulation further comprises an overcoat, wherein the overcoat comprises said at least one organic acid and at least one polymer. In another aspect, the amount of said at least one organic acid is from about 50 wt % to about 90 wt % of the overcoat. In another aspect, said organic acid is selected from lactic acid, phosphoric acid, citric acid, malic acid, fumaric acid, stearic acid, tartaric acid, benzoic acid, or combinations thereof. In another aspect, said at least one primary active substance is an addictive substance. In another aspect, the addictive substance is an opiod agonist and/or a narcotic analgesic.

In accordance with another aspect, there is provided a formulation comprising: at least one primary active substance; at least one coat comprising Eudragit E (dimethylaminoethyl methacrylate copolymer); and at least one coat comprising at least one acid to facilitate release of any active substance in the formulation.

In another aspect, a wt % ratio of said at least one acid to said at least one primary active substance is from about 1:100 to about 100:1. In another aspect, said at least one coat that comprises said Eudragit E further comprises at least one acid. In another aspect, said at least one coat that comprises said at least one acid is an overcoat. In another aspect, said at least one coat comprising at least one acid comprises from about 5 wt % to about 100 wt % by weight of said at least one organic acid based on the weight of said at least one coat. In another aspect, the overcoat comprises said at least one acid and at least one polymer. In another aspect, said at least one acid is at least one organic acid. In another aspect, said organic acid is selected from lactic acid, phosphoric acid, citric acid, malic acid, fumaric acid, stearic acid, tartaric acid, benzoic acid, or combinations thereof. In another aspect, the formulation is a controlled release formulation. In another aspect, onset of action of said at least one primary active substance is potentiated by the presence of a loading dose comprising said at least one primary active substance. In another aspect, the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release. In another aspect, prior to the POD, there is no significant controlled release. In another aspect, the formulation further comprises at least one secondary active substance, wherein the formulation has a quick onset of action of said at least one primary active substance followed by a controlled release of at least one secondary active substance, or vice versa, wherein said at least one primary active substance and said at least one secondary active substance are the same or different. In another aspect, the formulation further comprises at least one secondary active substance, wherein said at least one primary active substance in the loading dose is released at a higher rate in comparison to another dose having said at least one secondary active substance, wherein said at least one primary active substance and said at least one secondary active substance are the same or different. In another aspect, the formulation further comprises a core, wherein said at least one primary active substance is incorporated into the core, external to the core, or a combination thereof. In another aspect, the formulation further comprises a maintenance dose having at least one secondary active substance, wherein said at least one secondary active substance is the same or different than said at least one primary active substance. In another aspect, the maintenance dose comprises said at least one secondary active substance in a controlled release matrix. In another aspect, the formulation comprises at least one layer of said at least one loading dose and at least one layer of said at least one maintenance dose. In another aspect, said at least one layer of said at least one loading dose covers at least a portion of said at least one layer of said at least one maintenance dose or vice versa, forming a layered formulation. In another aspect, said at least one coat that comprises said Eudragit E and said at least one coat that comprises said at least one acid surrounds said layered formulation in any order. In another aspect, the formulation further comprises a core having said at least one maintenance dose and at least one coat comprising said at least one loading dose. In another aspect, the formulation further comprises at least one coat comprising at least one maintenance dose, which said at least one maintenance dose in the core is the same or different than said maintenance dose in said at least one coat. In another aspect, the core further comprises said at least one loading dose and/or said at least one coat of said loading dose further comprises said at least one maintenance dose, which said at least one loading dose in the core is the same or different than said loading dose in said at least one coat. In another aspect, said at least one coat comprising said at least one loading dose significantly covers said core. In another aspect, the formulation further comprises a core, wherein said at least one loading dose and said at least one maintenance dose are external to the core. In another aspect, said at least one coat that comprises said Eudragit E and/or said at least one coat that comprises said at least one acid further comprises at least one active substance, wherein said at least one active substance and said at least one primary active substance are the same or different. In another aspect, said at least one coat that comprises said Eudragit E, controls the release of said at least one primary active substance. In another aspect, release of any active substance in the formulation is activated by a pH dependent mechanism, ion-exchange dependent mechanism, bacterial flora/enzymes dependent mechanism, or a combination thereof. In another aspect, the Eudragit E comprises Eudragit E 100™. In another aspect, up to about 55% of the total dose is released as a loading dose to manage pain. In another aspect, the loading dose is released within about 60 minutes of ingestion. In another aspect, the formulation is configured such that when the formulation is administered in a physically compromised form to a subject, the rate of release of said at least one primary active substance in the loading dose is substantially the same or lower than the rate of release of said at least one primary active substance in the loading dose when the formulation is administered in an intact form. In another aspect, when the formulation is intact and or pulverized/milled and added to an alcoholic and/or non-alcoholic beverage, the rate of release of said at least one primary active substance in the loading dose is substantially the same or lower than the rate of release of said at least one primary active substance in the loading dose when the formulation is administered in an intact form. In another aspect, the beverage is an alcoholic beverage. In another aspect, the formulation comprises at least one excipient, wherein dissolution of the intact and or pulverized/milled formulation in alcoholic and/or non-alcoholic beverages causes the formulation to agglomerate. In another aspect, the at least one excipient comprises at least one sweliable material in such an amount that dissolution of the intact and or pulverized/milled formulation in alcoholic and/or non-alcoholic beverages causes the formulation to agglomerate. In another aspect, said at least one swellable material is selected from Eudragit RL, Eudragit NE, Eudragit RS and/or Eudragit NM. In another aspect, the at least one excipient comprises polyethylene oxide and Eudragit RL. In another aspect, the formulation is objectionable to chewing, sucking, licking and/or holding in the mouth. In another aspect, the formulation further comprises a bittering agent and/or irritant. In another aspect, the formulation is an oral formulation. In another aspect, the formulation is a solid unit form. In another aspect, the surface area covered by the Eudragit E in said at least one coat is greater than 5 mg/cm². In another aspect, the surface area covered by the Eudragit E in the coat is from about 5 mg/cm² to about 100 mg/cm². In another aspect, the formulation is capable of withstanding about a 350 N force. In another aspect, the formulation is effective in preventing significant dose dumping in any beverage. In another aspect, said at least one primary active substance is an addictive substance. In another aspect, the addictive substance is an opiod agonist and/or a narcotic analgesic. In another aspect, the loading dose is made from hot melt extrusion. In another aspect, at least one of the components used in the maintenance dose and/or the loading dose are made from hot melt extrusion. In another aspect, the maintenance dose and/or the loading dose are made from hot melt extrusion. In another aspect, an extruded component from the hot melt extrusion is extruded and cut into a desired shape and/or is extruded, ground and pressed. In another aspect, the particle size of the components of the formulation is less than about 1000 microns. In another aspect, the surface area of the components is between 0.5 and 10000 m²/g or higher. In another aspect, the formulation does not dose dump in the presence of alcohol.

In another aspect, less than about 30% by weight of the dose is released as a vapor for inhalation when the formulation is subjected to heat. In another aspect, less than about 10% by weight of the dose is released as a vapor for inhalation when the formulation is subjected to heat. In another aspect, the formulation is milled prior to heating. In another aspect, heating is achieved with an open flame or other heat source. In another aspect, the temperature for heating the formulation is about 540° C. In another aspect, less than about 20% of the dose is released after microwaving for about 2 minutes and thereafter, exposing the microwaved formulation to aqueous media.

In another aspect, there is provided use of the formulation for releasing up to about 55% of the total dose as a loading dose to manage an ailment. In another aspect, use of a controlled release narcotic analgesic having a loading dose for treatment of an ailment. In another aspect, the ailment is pain. In another aspect, the loading dose is released within about 60 minutes of ingestion.

In another aspect, there is provided a method of managing an ailment comprising administering the formulation for releasing up to about 55% of the total dose as a loading dose to manage the ailment. In another aspect, a method for treatment of an ailment comprising administering an oral controlled release formulation for releasing up to about 55% of the total dose as a loading dose, wherein the loading dose comprises at least one active substance. In another aspect, a method for treatment of an ailment comprising administering an oral controlled release formulation for releasing up to about 55% of the total dose as a loading dose, wherein the loading dose comprises at least one active substance. In another aspect, the ailment is pain.

In another aspect, there is provided an oral controlled release formulation for treatment of an ailment releasing up to about 55% of the total dose as a loading dose to treat the ailment, wherein the loading dose comprises at least one active substance. In another aspect, there is provided a formulation comprising at least one maintenance dose and at least one loading dose in at least one unit formulation for treatment of an ailment. In another aspect, the ailment is pain.

In other aspects:

1. (Claim 1) A formulation comprising:

at least one primary active substance; and

at least one coat comprising Eudragit E (dimethylaminoethyl methacrylate copolymer),

wherein the formulation is free of any active substance external to said at least one coat.

2. The formulation according to claim 1, wherein onset of action of said at least one primary active substance is potentiated by the presence of a loading dose comprising said at least one primary active substance.
3. The formulation according to claim 2, wherein the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release.
4. The formulation according to claim 3, wherein prior to the POD, there is no significant controlled release.
5. The formulation according to claim 2, wherein the formulation further comprises at least one secondary active substance, wherein the formulation has a quick onset of action of said at least one primary active substance followed by a controlled release of at least one secondary active substance, or vice versa, wherein said at least one primary active substance and said at least one secondary active substance are the same or different.
6. The formulation according to claim 2, wherein the formulation further comprises at least one secondary active substance, wherein said at least one primary active substance in the loading dose is released at a higher rate in comparison to another dose having said at least one secondary active substance, wherein said at least one primary active substance and said at least one secondary active substance are the same or different.
7. The formulation according to any one of claims 1 to 6 further comprising a core, wherein said at least one primary active substance is incorporated into the core, external to the core, or a combination thereof.
8. The formulation according to claim 2, wherein the formulation further comprises a maintenance dose having at least one secondary active substance, wherein said at least one secondary active substance is the same or different than said at least one primary active substance.
9. The formulation according to claim 8, wherein the maintenance dose comprises said at least one secondary active substance in a controlled release matrix.
10. The formulation according to claim 8 or 9, wherein the formulation comprises at least one layer of said at least one loading dose and at least one layer of said at least one maintenance dose.
11. The formulation according to claim 10 wherein said at least one layer of said at least one loading dose covers at least a portion of said at least one layer of said at least one maintenance dose or vice versa, forming a layered formulation.
12. The formulation according to claim 11, wherein said at least one coat surrounds said layered formulation.
13. The formulation according to claim 8 or 9 further comprises a core having said at least one maintenance dose and at least one coat comprising said at least one loading dose.
14. The formulation according to claim 13 further comprises at least one coat comprising at least one maintenance dose, which said at least one maintenance dose in the core is the same or different than said maintenance dose in said at least one coat.
15. The formulation according to claim 13 or 14, wherein the core further comprises said at least one loading dose and/or said at least one coat of said loading dose further comprises said at least one maintenance dose, which said at least one loading dose in the core is the same or different than said loading dose in said at least one coat.
16. The formulation according to claim 13, wherein said at least one coat comprising said at least one loading dose significantly covers said core.
17. The formulation according to claim 8 or 9 further comprises a core, wherein said at least one loading dose and said at least one maintenance dose are external to the core.
18. The formulation according to any one of claims 1 to 17, wherein said at least one coat comprising the Eudragit E further comprises at least one active substance, wherein said at least one active substance and said at least one primary active substance are the same or different.
19. The formulation according to claim 1, wherein said at least one coat controls the release of said at least one primary active substance.
20. The formulation according to claim 19, wherein release of any active substance in the formulation is activated by a pH dependent mechanism, ion-exchange dependent mechanism, bacterial flora/enzymes dependent mechanism, or a combination thereof.
21. The formulation according to claim 20, wherein the pH for the pH dependent mechanism is at most about 5.
22. The formulation according to claim 20, wherein the ion-exchange mechanism is controlled by at least one ion-exchange resin.
23. The formulation according to claim 22, wherein said at least one ion-exchange resin is selected from Cholestyramine, Colestipol, Sodium polystyrene sulfonate, Polacrilex resin, and/or Polacrilin potassium.
24. The formulation according to claim 20 wherein the bacterial flora/enzymes dependent mechanism is controlled by at least one polymer reactive to intestinal bacterial flora/enzymes.
25. The formulation according to claim 24 wherein said at least polymer is selected from polysaccharides such as guar gum, inulin, chondrotin sulphate, alginates, and/or dextran.
26. The formulation according to any one of claims 1 to 25, wherein the Eudragit E comprises Eudragit E 100™.
27. The formulation according to claim 1, wherein up to about 55% of the total dose is released as a loading dose to manage pain.
28. The formulation according to claim 27 wherein the loading dose is released within about 60 minutes of ingestion.
29. The formulation according to claim 2, wherein the formulation is configured such that when the formulation is administered in a physically compromised form to a subject, the rate of release of said at least one primary active substance in the loading dose is substantially the same or lower than the rate of release of said at least one primary active substance in the loading dose when the formulation is administered in an intact form.
30. The formulation according to claim 2, wherein when the formulation is pulverized/milled and added to an alcoholic and/or non-alcoholic beverage, the rate of release of said at least one primary active substance in the loading dose is substantially the same or lower than the rate of release of said at least one primary active substance in the loading dose when the formulation is administered in an intact form.
31. The formulation according to claim 30 wherein the beverage is an alcoholic beverage.
32. The formulation according to any one of claims 1 to 28, wherein the formulation comprises at least one excipient, wherein dissolution of the pulverized/milled formulation in alcoholic and/or non-alcoholic beverages causes the formulation to agglomerate.
33. The formulation according to claim 32 wherein the at least one excipient comprises at least one swellable material in such an amount that dissolution of the pulverized/milled formulation in alcoholic and/or non-alcoholic beverages causes the formulation to agglomerate.
34. The formulation according to claim 33 wherein said at least one swellable material is at least one pH independent polymer.
35. The formulation according to claim 33 wherein said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state.
36. The formulation according to claim 35 wherein said at least one swellable material comprises hydrophobic polymers.
37. The formulation according to claim 36 wherein the hydrophobic polymers are selected from Eudragit RL, Eudragit NE, Eudragit RS and/or Eudragit NM.
38. The formulation according to claim 37 wherein the at least one excipient comprises polyethylene oxide and Eudragit RL.
39. The formulation according to claim 8 further comprises at least one swellable material in such an amount that dissolution of the pulverized/milled formulation in alcoholic and/or non-alcoholic beverages causes the formulation to agglomerate, the amount of swellable material ranges from about 15 wt % to about 90 wt % of the maintenance dose and/or loading dose.
40. The formulation according to any one of claims 1 to 39, wherein the formulation is objectionable to chewing, sucking, licking and/or holding in the mouth.
41. The formulation according to claim 39 further comprises a bittering agent and/or irritant
42. The formulation according to any one of claims 1 to 40, wherein the formulation is an oral formulation.
43. The formulation according to any one of claims 1 to 42, wherein the formulation is a solid unit form.
44. The formulation according to any one of claims 1 to 43, wherein the surface area covered by the Eudragit E in said at least one coat is greater than 5 mg/cm².
45. The formulation according to claim 43 wherein the surface area covered by the Eudragit E in the coat is greater than 10 mg/cm².
46. The formulation according to claim 43 wherein the surface area covered by the Eudragit E in the coat is greater than 20 mg/cm².
47. The formulation according to claim 43 wherein the surface area covered by the Eudragit E in the coat is from about 5 mg/cm² to about 100 mg/cm².
48. The formulation according to claim 43 wherein the surface area covered by the Eudragit E in the coat is from about 10 mg/cm² to about 100 mg/cm².
49. The formulation according to claim 43 wherein the surface area covered by the Eudragit E in the coat is from about 20 mg/cm² to about 100 mg/cm².
50. The formulation according to any one of claims 1 to 49, wherein the formulation is capable of withstanding about a 350 N force.
51. The formulation according to any one of claims 1 to 50, wherein the formulation is effective in preventing significant dose dumping in any beverage.
52. The formulation according to any one of claims 1 to 50 further comprises at least one acid to facilitate release of any active substance in the formulation.
53. The formulation according to claim 8 further comprises at least one organic acid to facilitate release of any active substance in the formulation, wherein at least one of said at least one loading dose, said at least one maintenance dose, or said at least one coat comprises said at least one organic acid.
54. The formulation according to claim 53 wherein at least one of said at least one loading dose and said at least one coat comprises said at least one organic acid and the wt % ratio of the organic acid to said at least one primary active substance is from about 1:100 to about 100:1.
55. The formulation according to claim 53 or 54, wherein said at least one loading dose comprises from about 1 wt % to about 15 wt % by weight of said at least one organic acid based on the weight of the loading dose.
56. The formulation according to any one of claims 53 to 55, wherein said at least one maintenance dose comprises from about 1 wt % to about 10 wt % by weight of said at least one organic acid based on the weight of the maintenance dose.
57. The formulation according to any one of claims 53 to 56, wherein said at least one coat comprises from about 5 wt % to about 100 wt % by weight of said at least one organic acid based on the weight of said at least one coat.
58. The formulation according to any one of claims 54 to 57 further comprises an overcoat, wherein the overcoat comprises said at least one organic acid and at least one polymer.
59. The formulation according to claim 58 wherein the amount of said at least one organic acid is from about 5 wt % to less than about 100 wt % of the overcoat.
60. The formulation according to any one of claims 52 to 59, wherein said organic acid is selected from lactic acid, phosphoric acid, citric acid, malic acid, fumaric acid, stearic acid, tartaric acid, benzoic acid, or combinations thereof.
61. The formulation according to any one of claims 1 to 60, wherein said at least one primary active substance is an addictive substance.
62. The formulation according to claim 61 wherein the addictive substance is an opiod agonist and/or a narcotic analgesic.
63. (Claim 63) A formulation comprising:

a loading dose having at least one primary active substance, wherein the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release.

64. The formulation according to claim 63 wherein the onset of action is a quick onset of action.
65. The formulation according to claim 63 wherein the loading dose represents an active substance that is released at a higher rate in comparison to another dose of an active substance in the same formulation, wherein said at least one primary active substance and said active substance are the same or different.
66. The formulation according to claim 63 wherein a greater amount of said at least one primary active substance is released in a certain time interval in comparison to another dose of an active substance in the formulation that is released in a similar time interval, wherein said at least one primary active substance and said active substance are the same or different.
67. The formulation according to claim 63 or 64, wherein the formulation further comprises at least one secondary active substance, wherein the formulation has the onset of action of said at least one primary active substance followed by the controlled release of at least one secondary active substance, or vice versa, wherein said at least one primary active substance and said at least one secondary active substance are the same or different.
68. The formulation according to any one of claims 63 to 67, wherein prior to the POD, there is no significant controlled release.
69. The formulation according to any one of claims 63 to 68 further comprising a core, wherein said at least one primary active substance is incorporated into the core, external to the core, or a combination thereof.
70. The formulation according to claim 63 wherein the formulation further comprises a maintenance dose having at least one secondary active substance, wherein said at least one secondary active substance is the same or different than said at least one primary active substance.
71. The formulation according to claim 70 wherein the maintenance dose comprises said at least one secondary active substance in a controlled release matrix.
72. The formulation according to claim 70 or 71, wherein the formulation comprises at least one layer of said at least one loading dose and at least one layer of said at least one maintenance dose.
73. The formulation according to claim 72 wherein said at least one layer of said at least one loading dose covers at least a portion of said at least one layer of said at least one maintenance dose or vice versa, forming a layered formulation.
74. The formulation according to claim 73 wherein at least one coat surrounds said layered formulation.
75. The formulation according to claim 70 or 71 further comprises a core having said at least one maintenance dose and at least one coat comprising said at least one loading dose.
76. The formulation according to claim 75 further comprises at least one coat comprising at least one maintenance dose, which said at least one maintenance dose in the core is the same or different than said maintenance dose in said at least one coat.
77. The formulation according to claim 75 or 76, wherein the core further comprises said at least one loading dose and/or said at least one coat of said loading dose further comprises said at least one maintenance dose, which said at least one loading dose in the core is the same or different than said loading dose in said at least one coat.
78. The formulation according to claim 75, wherein said at least one coat comprising said at least one loading dose significantly covers said core.
79. The formulation according to claim 70 or 71 further comprises a core, wherein said at least one loading dose and said at least one maintenance dose are external to the core.
80. The formulation according to any one of claims 63 to 79, wherein the release of any active substance is activated by a pH dependent mechanism, ion-exchange dependent mechanism, bacterial flora/enzymes dependent mechanism, or a combination thereof.
81. The formulation according to claim 80 wherein the pH for the pH dependent mechanism is at most about 5.
82. The formulation according to claim 80 wherein the ion-exchange mechanism is controlled by at least one ion-exchange resin.
83. The formulation according to claim 82 wherein said at least one ion-exchange resin is selected from Cholestyramine, Colestipol. Sodium polystyrene sulfonate, Polacnlex resin, and/or Polacrilin potassium.
84. The formulation according to claim 80 wherein the bacterial flora/enzymes dependent mechanism is controlled by at least one polymer reactive to intestinal bacterial flora/enzymes.
85. The formulation according to claim 84 wherein said at least polymer is selected from polysaccharides such as guar gum, inulin, chondrotin sulphate, alginates, and/or dextran.
86. The formulation according to any one of claims 63 to 85, wherein the formulation comprises at least one excipient, wherein dissolution of the pulverized/milled formulation in alcoholic and/or non-alcoholic beverages causes the formulation to agglomerate.
87. The formulation according to claim 86 wherein the at least one excipient comprises at least one swellable material in such an amount that dissolution of the pulverized/milled formulation in alcoholic and/or non-alcoholic beverages causes the formulation to agglomerate.
88. The formulation according to claim 87 wherein said at least one sweliable material is at least one pH independent polymer.
89. The formulation according to claim 87 wherein said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state.
90. The formulation according to claim 87 wherein said at least one swellable material comprises hydrophobic polymers.
91. The formulation according to claim 90 wherein the hydrophobic polymers are selected from Eudragit RL, Eudragit NE, Eudragit RS and/or Eudragit NM.
92. The formulation according to claim 87 wherein the at least one excipient comprises polyethylene oxide and Eudragit RL.
93. The formulation according to claim 70 further comprises at least one swelable material in such an amount that dissolution of the pulverized/milled formulation in alcoholic and/or non-alcoholic beverages causes the formulation to agglomerate, the amount of swellable material ranges from about 15 wt % to about 90 wt % of the maintenance dose and/or loading dose.
94. The formulation according to any one of claims 63 to 93 further comprises at least one acid to facilitate release of any active substance in the formulation.
95. The formulation according to claim 70 further comprises at least one organic acid to facilitate release of any active substance in the formulation, wherein at least one of said at least one loading dose or said at least one maintenance dose comprises said at least one organic acid.
96. The formulation according to claim 95 wherein said at least one loading dose comprises from about 1 wt % to about 15 wt % by weight of said at least one organic acid based on the weight of the loading dose.
97. The formulation according to claim 95 or 96, wherein said at least one maintenance dose comprises from about 1 wt % to about 10 wt % by weight of said at least one organic acid based on the weight of the maintenance dose.
98. The formulation according to claim 95 or 96, wherein said at least one loading dose comprises said at least one organic acid.
99. The formulation according to any one of claims 94 to 97 further comprises an overcoat, wherein the overcoat comprises said at least one organic acid and at least one polymer.
100. The formulation according to claim 99 wherein the amount of said at least one organic acid is from about 5 wt % to less than about 100 wt % of the overcoat.
101. The formulation according to any one of claims 94 to 100, wherein said organic acid is selected from lactic acid, phosphoric acid, citric acid, malic acid, fumaric acid, stearic acid, tartaric acid, benzoic acid, or combinations thereof.
102. The formulation according to any one of claims 63 to 101, wherein said at least one primary active substance is an addictive substance.
103. The formulation according to claim 102, wherein the addictive substance is an opiod agonist and/or a narcotic analgesic.
104. The formulation according to any one of claims 63 to 103, wherein up to about 55% of the total dose is released as a loading dose to manage pain.
105. The formulation according to claim 104, wherein the loading dose is released within about 60 minutes of ingestion.
106. (Claim 106) A formulation comprising:

at least one primary active substance; and

at least one excipient, wherein dissolution of the intact and or pulverized/milled formulation in alcoholic and/or non-alcoholic beverages causes the formulation to agglomerate.

107. The formulation according to claim 106, wherein the at least one excipient comprises at least one swellable material in such an amount that dissolution of the pulverized/milled formulation in alcoholic and/or non-alcoholic beverages causes the formulation to agglomerate.
108. The formulation according to claim 106, wherein said at least one swellable material is at least one pH independent polymer.
109. The formulation according to claim 106, wherein said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state.
110. The formulation according to claim 106, wherein said at least one swellable material comprises hydrophobic polymers.
111. The formulation according to claim 110, wherein the hydrophobic polymers are selected from Eudragit RL, Eudragit NE, Eudragit RS and/or Eudragit NM.
112. The formulation according to claim 107, wherein the at least one excipient comprises polyethylene oxide and Eudragit RL.
113. The formulation according to any one of claims 106 to 112 further comprises at least one organic acid to facilitate release of any active substance in the formulation.
114. The formulation according to claim 113 further comprises an overcoat, wherein the overcoat comprises said at least one organic acid and at least one polymer.
115. The formulation according to claim 114, wherein the amount of said at least one organic acid is from about 50 wt % to about 90 wt % of the overcoat.
116. The formulation according to any one of claims 113 to 115, wherein said organic acid is selected from lactic acid, phosphoric acid, citric acid, malic acid, fumaric acid, stearic acid, tartaric acid, benzoic acid, or combinations thereof.
117. The formulation according to any one of claims 106 to 116, wherein said at least one primary active substance is an addictive substance.
118. The formulation according to claim 117, wherein the addictive substance is an oplod agonist and/or a narcotic analgesic.
119. (Claim 119) A formulation comprising:

at least one primary active substance;

at least one coat comprising Eudragit E (dimethylaminoethyl methacrylate copolymer); and

at least one coat comprising at least one acid to facilitate release of any active substance in the formulation.

120. The formulation according to claim 119, wherein a wt % ratio of said at least one acid to said at least one primary active substance is from about 1:100 to about 100:1.
121. The formulation according to claim 119 or 120, wherein said at least one coat that comprises said Eudragit E further comprises at least one acid.
122. The formulation according to any one of claims 119 to 121, wherein said at least one coat that comprises said at least one acid is an overcoat.
123. The formulation according to claim 122, wherein said at least one coat comprising at least one acid comprises from about 5 wt % to about 100 wt % by weight of said at least one organic acid based on the weight of said at least one coat.
124. The formulation according to claim 122, wherein the overcoat comprises said at least one acid and at least one polymer.
125. The formulation according to any one of claims 119 to 124, wherein said at least one acid is at least one organic acid.
126. The formulation according to claim 125, wherein said organic acid is selected from lactic acid, phosphoric acid, citric acid, malic acid, fumaric acid, stearic acid, tartaric acid, benzoic acid, or combinations thereof.
127. The formulation according to any one of claims 119 to 128, wherein the formulation is a controlled release formulation.
128. The formulation according to any one of claims 119 to 126, wherein onset of action of said at least one primary active substance is potentiated by the presence of a loading dose comprising said at least one primary active substance.
129. The formulation according to claim 128, wherein the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release.
130. The formulation according to claim 129, wherein prior to the POD, there is no significant controlled release.
131. The formulation according to claim 128, wherein the formulation further comprises at least one secondary active substance, wherein the formulation has a quick onset of action of said at least one primary active substance followed by a controlled release of at least one secondary active substance, or vice versa, wherein said at least one primary active substance and said at least one secondary active substance are the same or different.
132. The formulation according to claim 128, wherein the formulation further comprises at least one secondary active substance, wherein said at least one primary active substance in the loading dose is released at a higher rate in comparison to another dose having said at least one secondary active substance, wherein said at least one primary active substance and said at least one secondary active substance are the same or different.
133. The formulation according to any one of claims 119 to 132 further comprising a core, wherein said at least one primary active substance is incorporated into the core, external to the core, or a combination thereof.
134. The formulation according to claim 128, wherein the formulation further comprises a maintenance dose having at least one secondary active substance, wherein said at least one secondary active substance is the same or different than said at least one primary active substance.
135. The formulation according to claim 134, wherein the maintenance dose comprises said at least one secondary active substance in a controlled release matrix.
136. The formulation according to claim 134 or 135, wherein the formulation comprises at least one layer of said at least one loading dose and at least one layer of said at least one maintenance dose.
137. The formulation according to claim 136, wherein said at least one layer of said at least one loading dose covers at least a portion of said at least one layer of said at least one maintenance dose or vice versa, forming a layered formulation.
138. The formulation according to claim 137, wherein said at least one coat that comprises said Eudragit E and said at least one coat that comprises said at least one acid surrounds said layered formulation in any order.
139. The formulation according to claim 134 or 135 further comprises a core having said at least one maintenance dose and at least one coat comprising said at least one loading dose.
140. The formulation according to claim 139 further comprises at least one coat comprising at least one maintenance dose, which said at least one maintenance dose in the core is the same or different than said maintenance dose in said at least one coat.
141. The formulation according to claim 139 or 140, wherein the core further comprises said at least one loading dose and/or said at least one coat of said loading dose further comprises said at least one maintenance dose, which said at least one loading dose in the core is the same or different than said loading dose in said at least one coat.
142. The formulation according to claim 139, wherein said at least one coat comprising said at least one loading dose significantly covers said core.
143. The formulation according to claim 139 or 140 further comprises a core, wherein said at least one loading dose and said at least one maintenance dose are external to the core.
144. The formulation according to any one of claims 119 to 143, wherein said at least one coat that comprises said Eudragit E and/or said at least one coat that comprises said at least one acid further comprises at least one active substance, wherein said at least one active substance and said at least one primary active substance are the same or different.
145. The formulation according to claim 119, wherein said at least one coat that comprises said Eudragit E, controls the release of said at least one primary active substance.
146. The formulation according to claim 145, wherein release of any active substance in the formulation is activated by a pH dependent mechanism, ion-exchange dependent mechanism, bacterial flora/enzymes dependent mechanism, or a combination thereof.
147. The formulation according to any one of claims 119 to 146, wherein the Eudragit E comprises Eudragit E 100™.
148. The formulation according to claim 119, wherein up to about 55% of the total dose is released as a loading dose to manage pain.
149. The formulation according to claim 148, wherein the loading dose is released within about 60 minutes of ingestion.
150. The formulation according to claim 128, wherein the formulation is configured such that when the formulation is administered in a physically compromised form to a subject, the rate of release of said at least one primary active substance in the loading dose is substantially the same or lower than the rate of release of said at least one primary active substance in the loading dose when the formulation is administered in an intact form.
151. The formulation according to claim 128, wherein when the formulation is intact and or pulverized/milled and added to an alcoholic and/or non-alcoholic beverage, the rate of release of said at least one primary active substance in the loading dose is substantially the same or lower than the rate of release of said at least one primary active substance in the loading dose when the formulation is administered in an intact form.
152. The formulation according to claim 151, wherein the beverage is an alcoholic beverage.
153. The formulation according to any one of claims 119 to 152, wherein the formulation comprises at least one excipient, wherein dissolution of the intact and or pulverized/milled formulation in alcoholic and/or non-alcoholic beverages causes the formulation to agglomerate.
154. The formulation according to claim 153, wherein the at least one excipient comprises at least one swellable material in such an amount that dissolution of the intact and or pulverized/milled formulation in alcoholic and/or non-alcoholic beverages causes the formulation to agglomerate.
155. The formulation according to claim 154, wherein said at least one swellable material is selected from Eudragit RL, Eudragit NE, Eudragit RS and/or Eudragit NM.
156. The formulation according to claim 154, wherein the at least one excipient comprises polyethylene oxide and Eudragit RL.
157. The formulation according to any one of claims 119 to 156, wherein the formulation is objectionable to chewing, sucking, licking and/or holding in the mouth.
158. The formulation according to claim 157 further comprises a bittering agent and/or irritant.
159. The formulation according to any one of claims 119 to 158, wherein the formulation is an oral formulation.
160. The formulation according to any one of claims 119 to 159, wherein the formulation is a solid unit form.
161. The formulation according to any one of claims 119 to 160, wherein the surface area covered by the Eudragit E in said at least one coat is greater than 5 mg/cm².
162. The formulation according to claim 161, wherein the surface area covered by the Eudragit E in the coat is from about 5 mg/cm² to about 100 mg/cm².
163. The formulation according to any one of claims 119 to 162, wherein the formulation is capable of withstanding about a 350 N force.
164. The formulation according to any one of claims 119 to 163, wherein the formulation is effective in preventing significant dose dumping in any beverage.
165. The formulation according to any one of claims 119 to 164, wherein said at least one primary active substance is an addictive substance.
166. The formulation according to claim 165, wherein the addictive substance is an opiod agonist and/or a narcotic analgesic.
167. The formulation according to any one of claims 2, 63 and 128, wherein the loading dose is made from hot melt extrusion.
168. The formulation according to any one of claims 1 to 167, wherein at least one of the components used in the maintenance dose and/or the loading dose are made from hot melt extrusion.
169. The formulation according to claim 168, wherein the maintenance dose and/or the loading dose are made from hot melt extrusion.
170. The formulation according to any one of claims 167 to 169, wherein an extruded component from the hot melt extrusion is extruded and cut into a desired shape and/or is extruded, ground and pressed.
171. The formulation according to any one of claims 1 to 170, wherein the particle size of the components of the formulation is less than about 1000 microns.
172. The formulation according to any one of claims 1 to 171, wherein the surface area of the components is between 0.5 and 10000 m²/g or higher.
173. The formulation according to any one of claims 1 to 172, wherein the formulation does not dose dump in the presence of alcohol.
174. The formulation according to any one of claims 1 to 172, wherein less than about
30% by weight of the dose is released as a vapor for inhalation when the formulation is subjected to heat.
175. The formulation according to claim 174, wherein less than about 10% by weight of the dose is released as a vapor for inhalation when the formulation is subjected to heat.
176. The formulation according to claim 174 or 175, wherein the formulation is milled prior to heating.
177. The formulation according to any one of claims 174 to 176, wherein heating is achieved with an open flame or other heat source.
178. The formulation according to any one of claims 174 to 177, wherein the temperature for heating the formulation is about 540° C.
179. The formulation according to any one of claims 1 to 172, wherein less than about
20% of the dose is released after microwaving for about 2 minutes and thereafter, exposing the microwaved formulation to aqueous media.
180. Use of the formulation according to any one of claims 1 to 179 for releasing up to about 55% of the total dose as a loading dose to manage an ailment.
181. Use of a controlled release narcotic analgesic having a loading dose for treatment of an ailment.
182. The use according to claim 180 or 181, wherein the ailment is pain.
183. The use according to any one of claims 180 to 182, wherein the loading dose is released within about 60 minutes of ingestion.
184. A method of managing an ailment comprising administering the formulation according to any one of claims 1 to 175 for releasing up to about 55% of the total dose as a loading dose to manage the ailment.
185. (Claim 185) A method for treatment of an ailment comprising administering an oral controlled release formulation for releasing up to about 55% of the total dose as a loading dose, wherein the loading dose comprises at least one active substance.
186. (Claim 186) A method for treatment of an ailment comprising administering an oral controlled release formulation for releasing up to about 55% of the total dose as a loading dose, wherein the loading dose comprises at least one active substance.
187. The method according to any one of claims 184 to 186, wherein the ailment is pain.
188. (Claim 188) An oral controlled release formulation for treatment of an ailment releasing up to about 55% of the total dose as a loading dose to treat the ailment, wherein the loading dose comprises at least one active substance.
189. (Claim 189) A formulation comprising at least one maintenance dose and at least one loading dose in at least one unit formulation for treatment of an ailment.
190. The formulation according to claim 188 or 189, wherein the ailment is pain.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

Embodiments of the present invention will now be described, by way of example only, with reference to the attached Figures.

FIG. 1 shows the effect of subjecting the tablet of Example 7 to a 350 Newton force using a Vankel VK200 Tablet Hardness Tester;

FIG. 2 shows the effect of subjecting a commercially available Oxycodone extended release tablet to a 350 Newton force using a Vankel VK200 Tablet Hardness Tester.

FIG. 3 shows a pulverized/milled tablet of Example 7 compared to the pulverized/milled tablet of the commercially available Oxycodone extended release tablet shown in FIG. 2;

FIG. 4 shows the effect of subjecting the pulverized/milled Oxycodone tablet of Example 7 to moisture or aqueous media;

FIG. 5A shows a mean dissolution profile of an example of the commercially available Oxycodone extended release tablet in comparison to the Oxycodone tablets of Example 7 in acidic media;

FIG. 5B shows a mean dissolution profile of an example of the commercially available Oxymorphone extended release tablet in comparison to the Oxymorphone tablets of Example 5 in acidic media;

FIG. 6 shows an embodiment of the mechanism of action of certain examples of the tablets described herein;

FIG. 7 shows a mean dissolution profile of an example of the commercially available Oxycodone extended release tablet in comparison to the Oxycodone tablet of Example 7 in acidic media;

FIG. 8 shows a mean dissolution profile of Oxycodone tablets of Example 7 in media of varying pH;

FIG. 9 shows a mean dissolution profile of Oxymorphone tablets of Example 5 in alcoholic media;

FIG. 10 shows a mean dissolution profile of Oxycodone tablets of Example 7 in alcoholic media;

FIG. 11 shows a pulverized/milled Oxycodone tablets of Example 7 compared to the pulverized/milled tablet of the commercially available Oxycodone extended release tablet in Coca-Cola™;

FIG. 12 shows a pulverized/milled Oxycodone tablets of Example 7 compared to the pulverized/milled tablet of the commercially available Oxycodone extended release tablet in water;

FIG. 13 shows a mean dissolution of Oxycodone tablets of Example 29 in water; and

FIG. 14 shows a mean dissolution profile of Oxycodone tablets of the Examples described herein compared to the commercially available Oxycodone HCl extended release tablets, microwaved for 2 minutes in 0.1N HCl or in 40% Ethanol and 0.1N HCl.

### DETAILED DESCRIPTION OF CERTAIN EMBODIMENTS

### Definitions

The terms “formulation” and “composition” may be used interchangeably.

The term “active ingredient” or “active substance” means any compound which has biological, chemical, or physiological utility including, without limitation, active pharmaceutical ingredient, drug, naturally occurring compound, nucleic acid compound, peptide compound, biologics, nutraceutical, agricultural or nutritional ingredient or synthetic drug, Including addictive substances such as opiod agonists or narcotic analgesics.

The terms “primary” and “secondary” used in conjunction with “active Ingredient” were used to assist simply for antecedent purposes and are not meant to imply the level of importance of the active ingredient.

The term “addictive substance” means any compound upon which a user may develop a psychic or physical dependence, including, without limitation, any active ingredient or active substance as defined herein that may have this property.

Many interchangeable terms are commonly used to describe the psychic or physical dependence of people upon compounds. The term addiction is most commonly used when talking about the strong analgesics or opioid agonist or abuse-able substances. The strong analgesics or opioid agonist or abuse-able substances, in contrast to the weaker agents such as aspirin, acetaminophen, and the like, are employed in the relief of more severe pain. They usually produce a euphoric effect when crushed and swallowed, snorted and “shoot” parenterally. When taken as oral controlled release composition intact there is usually no significant euphoria.

Addictive substances also include drugs most commonly employed for illicit purposes (to bring about a “high”, euphoria, excitement, stupor, sleep deprivation etc.,) such as the barbiturates, lysergic acid diethylamide (LSD), mescaline, marijuana (tetrahydrocannabinol), heroin, and the like, the central nervous system stimulants (the amphetamines and the like) sedative, hypnotics and some of the major and minor tranquilizers (the promazines, meprobamate, the diazepines, and the like).

Examples of some of the opiod agonists or narcotic analgesics contemplated for use in this invention include alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tramadol, tilidine, alphaprodine, dextroporpoxyphene, propiram, profadol, phenampromide, thiambutene, pholcodeine, 3-trans-dimethylamino-4-phenyl-4-trans-carbethoxy-delta-cyclohexene, 3-dimethylamino-0-(4-methoxyphenylcarbamoyl)-propiophenone oxime, (−)β-2′-hydroxy-2,9-dimethyl-5-phenyl-6,7-benzomorphan. (−)2′-hydroxy-2-(3-methyl-2-butenyl)-9-methyl-5-phenyl-6,7-benzomorphan, pirinitramide, (−)α-5,9-diethyl-2′-hydroxy-2-methyl-6,7-benzomorphan, ethyl-1-(2-dmethylaminoethyl)-4,5,6,7-tetrahydro-3-methyl-4-oxo-6-phenylindole-2-carboxylate, 1-Benzoylmethyl-2,3-dimethyl-3-(m-hydroxyphenyl)-piperidine, N-allyl-7α-(1-(R)-hydroxy-1-methylbutyl)-6,14-endo-ethanotetrahydron ororipavine, (−)2′-hydroxy-2-methyl-6,7-benzomorphan, noracylmethadol, phenoperidine, α-dl-methadol, β-dl-methadol, α-1-methadol, β-dl-acetylmethadol, α-1-acetylmethadol and β-1-acetylmethadol and pharmaceutically acceptable salts thereof, stereolsomers thereof, ethers thereof, esters thereof, and mixtures thereof and their prodrugs in each case.

Furthermore, in certain embodiments, the formulations described herein may be particular suitable for preventing abuse of a pharmaceutical active ingredient selected from the group consisting of opiates, opioids, tranquilizers, typically benzodiazepines, barbiturates, stimulants and other narcotics and their prodrugs in each case. The formulations may be particularly suitable for preventing abuse of an opiate, opioid, tranquilizer or another narcotic selected from the group consisting of N-{1-[2-(4-ethyl-5-oxo-2-tetrazolin-1-yl)ethyl]-4-methoxymethyl-4-piperid-yl}propionanilide (alfentanil), 5,5-diallylbarbituric acid (allobarbital), allylprodine, alphaprodine, 8-chloro-1-methyl-6-phenyl-4H-[1,2,4]triazolo[4,3-a][1,4]-benzodiazepine (alprazolam), 2-diethylaminopropiophenone (amfepramone), (±)-α-methyl-phenethylamine (amphetamine), 2-α-methylphenethylamino)-2-phenylacetonitrile (amphetaminil), 5-ethyl-5-isopentylbarbituric acid (amobarbital), anileridine, apocodeine, 5,5-diethybarbituric acid (barbital), benzylmorphine, bezitramide, 7-bromo-5-(2-pyridyl)-1H-1,4-benzodiazepine-2(3H)-one (bromazepam), 2-bromo-4-(2-chlorophenyl)-9-methyl-6H-thieno[3,2-f][1,2,4]triazolo-[4,3-a][1,4]diazepine (brotizolam), 17-cyclopropylmethyl-4,5α-epoxy-7α[(S)-1-hydroxy-1,2,2-trimethyl-propyl]-6-methoxy-6,14-endo-ethanomorphinane-3-ol (buprenorphine), 5-butyl-5-ethylbarbituric acid (butobarbital), butorphanol, (7-chloro-1,3-dihydro-1-methyl-2-oxo-5-phenyl-2H-1,4-benzodiazepine-3-yl)-dimethylcarbamate (camazepam), (1S,2S)-2-amino-1-phenyl-1-propanol (cathine/D-norpseudoephedrine), 7-chloro-N-methyl-5-phenyl-3H-1,4-benzodiazepine-2-ylamine-4-oxide (chlorodiazepoxide), 7-chloro-1-methyl-5-phenyl-1H-1,5-benzodiazepine-2,4(3H,5H)-dione (clobazam), 5-(2-chlorophenyl)-7-nitro-1H-1,4-benzodiazepine-2(3H)-one (clonazepam), clonitazene, 7-chloro-2,3-dihydro-2-oxo-5-phenyl-1H-1,4-benzodiazepine-3-carboxylic acid (clorazepate), 5-(2-chlorophenyl)-7-ethyl-1-methyl-1H-thieno[2,3-e][1,4]diazepine-2(3H)-one (clotiazepam), 10-chloro-11b-(2-chlorophenyl)-2,3,7,11b-tetrahydrooxazolo[3,2-d][1,4]benzodiazepine-6(5H)-one (cloxazolam), (−)-methyl-[3β-benzoyloxy-2-β(1α(H,5-αH)-tropancarboxylate](cocaine), 4,5-α-epoxy-3-methoxy-17-methyl-7-morphinene-6-α-ol (codeine), 5-(1-cyclohexenyl)-5-ethylbarbituric acid (cyclobarbital), cyclorphan, cyprenorphine, 7-chloro-5-(2-chlorophenyl)-1H-1,4-benzodiazepine-2(3H)-one (delorazepam), desomorphine, dextromoramide, (+)-(1-benzyl-3-dimethylamino-2-methyl-1-phenylpropyl)propionate (dextropropoxyphen), dezocine, diampromide, diamorphone, 7-chloro-1-methyl-5-phenyl-1H-1,4-benzodiazepine-2(3H)-one (diazepam), 4,5-α-epoxy-3-methoxy-17-methyl-6-α-morphinanol (dihydrocodeine), 4,5-α-epoxy-17-methyl-3,6-α-morphinandiol (dihydromorphine), dimenoxadol, dimepheptanol, dimethylthiambutene, dioxaphetyl butyrate, dipipanone, (6aR,10aR)-6,6,9-trimethyl-3-pentyl-6a,7,8,10a-tetrahydro-6H-benzo[c]chromene-1-ol (dronabinol), eptazocine, 8-chloro-6-phenyl-4H-[1,2,4]triazolo[4,3-a][1,4]benzodiazepine (estazolam), ethoheptazine, ethylmethylthiambutene, ethyl[7-chloro-5-(2-fluorophenyl)-2,3-dihydro-2-oxo-1H-1,4-benzodiazepine-3-carboxylate](ethyl loflazepate), 4,5-α-epoxy-3-ethoxy-17-methyl-7-morphinene-6-α-ol (ethylmorphine), etonitazene, 4,5-α-epoxy-7-α-(1-hydroxy-1-methylbutyl)-6-methoxy-17-methyl-6,14-endo-etheno-morphinan-3-ol (etorphine). N-ethyl-3-phenyl-8,9,10-trinorbornan-2-ylamine (fencamfamine), 7-[2-(1-methyl-phenethylamino)ethyl]-theophylline) (fenethylline), 3-(α-methylphenethylamino)proponitrile (fenproporex), N-(1-phenethyl-4-piperidyl)propionanilide (fentanyl), 7-chloro-5-(2-fluorophenyl)-1-methyl-1H-1,4-benzodiazepine-2(3H)-one (fludiazepam), 5-(2-fluorophenyl)-1-methyl-7-nitro-1H-1,4-benzodiazepine-2(3H)-one (flunitrazepam), 7-chloro-1-(2-diethylaminoethyl)-5-(2-fluorophenyl)-1H-1,4-benzodiazepine-2(3H)-one (flurazepam), 7-chloro-5-phenyl-1-(2,2,2-trifluoroethyl)-1H-1,4-benzodiazepine-2(3H)-one (halazepam), 10-bromo-11b-(2-fluorophenyl)-2,3,7,11b-tetrahydro[1,3]oxazolyl[3,2-d][1,4]benzodiazepine-6(5H)-one (haloxazolam), heroin, 4,5-α-epoxy-3-methoxy-17-methyl-6-morphinanone (hydrocodone), 4,5-α-epoxy-3-hydroxy-17-methyl-6-morphinanone (hydromorphone), hydroxypethidine, isomethadone, hydroxymethyl morphinane, 11-chloro-8,12b-dihydro-2,8-dimethyl-12b-phenyl-4H-[1,3]oxazino[3,2-d][1,4]benzodiazepine-4,7(8H)-dione (ketazolam), 1-[4-(3-hydroxyphenyl)-1-methyl-4-piperidyl]-1-propanone (ketobemidone), (3S,6S)-6-dimethylamino-4,4-diphenylheptan-3-yl acetate (levacetylmethadol (LAAM)), (−)-6-dimethylamino-4,4-diphenol-3-heptanone (levomethadone), (−)-17-methyl-3-morphinanol (levorphanol), levophenacylmorphane, lofentanil, 6-(2-chlorophenyl)-2-(4-methyl-1-piperazinylmethylene)-8-nitro-2H-imidazo-[1,2-a][1,4]-benzodiazepine-1(4H)-one (loprazolam), 7-chloro-5-(2-chlorophenyl)-3-hydroxy-1H-1,4-benzodiazepine-2(3H)-one (lorazepam), 7-chloro-5-(2-chlorophenyl)-3-hydroxy-1-methy-1H-1,4-benzodiazepine-2(3H-)-one (lormetazepam), 5(4-chlorophenyl)-2,5-dihydro-3H-imidazo-[2,1-a]isoindol-5-ol (mazindol), 7-chloro-2,3-dihydro-1-methyl-5-phenyl-1H-1,4-benzodiazepine (medazepam), N-(3-chloropropyl)-α-methylphenethylamine (mefenorex), meperidine, 2-methyl-2-propyltrimethylene dicarbamate (meprobamate), meptazinol, metazocine, methylmorphine, N,α-dimethylphenethylamine (methamphetamine), (±)-6-dimethylamino-4,4-diphenyl-3-heptanone (methadone), 2-methyl-3-o-tolyl-4(3H)-quinazolinone (methaqualone), methyl [2-phenyl-2-(2-piperidyl)acetate](methylphenidate), 5-ethyl-1-methyl-5-phenylbarbituric acid (methylphenobarbital), 3,3-diethyl-5-methyl-2,4-piperidinedione (methyprylon), metopon, 8-chloro-6-(2-fluorophenyl)-1-methyl-4H-imidazo[1,5-a][1,4]benzodiazepine (midazolam), 2-(benzhydrylsulfinyl)-acetamide (modafinil), 4,5-α-epoxy-17-methyl-7-morphinen-3,6-α-diol (morphine), myrophine, (±)-trans-3-(1,1-dimethylheptyl)-7,8,10,10-α-tetrahydro-1-hydroxy-6,6-dimethyl-6H-dibenzo[b,d]pyrane-9 (6-αH)-one (nabilone), nalbuphine, nalorphine, narceine, nicomorphine, 1-methyl-7-nitro-5-phenyl-1H-1,4-benzodiazepine-2(3H)-one (nunetazepam), 7-nitro-5-phenyl-1H-1,4-benzodiazepine-2(3H)-one (nitrazepam), 7-chloro-5-phenyl-1H-1,4-benzodiazepine-2(3H)-one (nordazepam), norlevorphanol, 6-dimethytamino-4,4-diphenyl-3-hexanone (normethadone), normorphine, norpipanone, the exudation of plants belonging to the species Papaver somniferum (opium), 7-chloro-3-hydroxy-5-phenyl-1H-1,4-benzodiazepine-2(3H)-one (oxazepam), (cis-trans)-10-chloro-2,3,7,11b-tetrahydro-2-methyl-11b-phenyloxazolo[3,2-d][1,4]benzodiazepine-6-(5H)-one (oxazolam), 4,5-α-epoxy-14-hydroxy-3-methoxy-17-methyl-6-morphinanone (oxycodone), oxymorphone, plants and parts of plants belonging to the species Papaver somniferum (including the subspecies setigerum), papaveretum, 2-imino-5-phenyl-4-oxazolidinone (pemoline), 1,2,3,4,5,6-hexahydro-6,11-dimethyl-3-(3-methyl-2-butenyl)-2,6-methano-3-benzazocin-8-ol (pentazocine), 5-ethyl-5-(1-methylbutyl)-barbituric acid (pentobarbital), ethyl-(1-methyl-4-phenyl-4-piperidine carboxylate) (pethidine), phenadoxone, phenomorphan, phenazocine, phenoperidine, piminodine, pholcodine, 3-methyl-2-phenylmorpholine (phenmetrazine), 5-ethyl-5-phenylbarbituric acid (phenobarbital), α,α-dimethylphenethylamine (phentermine), 7-chloro-5-phenyl-1-(2-propynyl)-1H-1,4-benzodiazepine-2(3H)-one (pinazepam), α-(2-piperidyl)benzhydryl alcohol (pipradrol), 1′-(3-cyano-3,3-diphenylpropyl)[1,4′-bipiperidine]-4′-carboxamide (piritramide), 7-chloro-1-(cyclopropylmethyl)-5-phenyl-1H-1,4-benzodiazepine-2(3H)-one (prazepam), profadol, proheptazine, promedol, properidine, propoxyphene, N-(1-methyl-2-piperidinoethyl)-N-(2-pyridyl)propionamide, methyl [3-(4-methoxycarbonyl-4-(N-phenylpropanamido)piperidino]propanoate) (remifentanil), 5-sec-butyl-5-ethylbarbituric acid (secbutabarbital), 5-allyl-5-(1-methylbutyl)-barbituric acid (secobarbital), N-[4-methoxymethyl-1-[2-(2-thienyl)ethyl]-4-piperidyl]-propionamide (sufentanil), 7-chloro-2-hydroxy-methyl-5-phenyl-1H-1,4-benzodiazepin-2(3H)-one (temazepam), 7-chloro-5-(1-cyclohexenyl)-1-methyl-1H-1,4-benzodiazepine-2(3H)-one (tetrazepam), ethyl(2-dimethylamino-1-phenyl-3-cyclohexene-1-carboxylate) (tilidine (cis and trans)), tramadol, 8-chloro-6-(2-chlorophenyl)-1-methy-4H-[1,2,4]triazolo[4,3-a][1,4]benzodiazepine (triazolam), 5-(1-methylbutyl)-5-vinylbarbituric acid (vinylbital), (1R*,2R*)-3-(3-dimethylamino-1-ethyl-2-methyl-propyl)-phenol, (1R,2R,4S)-2-(dimethylamino)methy-4-(p-fluoro-benzyloxy)-1-(m-methoxyphenyl)cyclohexanol, (1R,2R)-3-(2-dimethylaminomethyl-cyclohexyl)phenol, (1S,2S)-3-(3-dimethylamino-1-ethyl-2-methyl-propyl)phenol, (2R,3R)-1-dimethylamino-3(3-methoxyphenyl)-2-methyl-pentan-3-ol, (1 RS,3RS,6RS)-6-dimethylaminomethyl-1-(3-methoxyphenyl)-cyclohexane-1,3-diol, 3-(2-dimethylaminomethyl-1-hydroxy-cyclohexyl)phenyl 2-(4-isobutoxy-phenyl)-propionate, 3-(2-dimethylaminomethyl-1-hydroxy-cyclohexyl)phenyl 2-(6-methoxy-naphthalen-2-yl)-propionate, 3-(2-dimethylamino-methyl-cyclohex-1-enyl)-phenyl 2-(4-isobutyl-phenyl)-propionate, 3-(2-dimethylaminomethyl-cyclohex-1-enyl)-phenyl 2-(6-methoxy-naphthalen-2-yl)-propionate, (RR-SS)-2-acetoxy-4-trifluoromethyl-benzoic acid 3-(2-dimethylaminomethyl-1-hydroxy-cyclohexyl)-phenyl ester, (RR-SS)-2-hydroxy-4-trifluoromethyl-benzoic acid 3-(2-dimethylaminomethyl-1-hydroxy-cyclohexyl)-phenyl ester, (RR-SS)-4-chloro-2-hydroxy-benzoic acid 3-(2-dimethylaminomethyl-1-hydroxy-cyclohexyl)-phenyl ester, (RR-SS)-2-hydroxy-4-methyl-benzoic acid 3-(2-dimethylaminomethyl-1-hydroxy-cyclohexyl)-phenyl ester, (RR-SS)-2-hydroxy-4-methoxy-benzoic acid 3-(2-dimethylaminomethyl-1-hydroxy-cyclohexyl)-phenyl ester, (RR-SS)-2-hydroxy-5-nitro-benzoic acid 3-(2-dimethylaminomethyl-1-hydroxy-cyclohexyl)-phenyl ester, (RR-SS)-2′,4′-difluoro-3-hydroxy-biphenyl-4-carboxylic acid 3-(2-dimethylaminomethyl-1-hydroxy-cyclohexyl)-phenyl ester and for corresponding stereoisomeric compounds, the corresponding derivatives thereof in each case, in particular esters or ethers, and the physiologically acceptable compounds thereof in each case, in particular the salts and solvates thereof, and their prodrugs in each case. The compounds (1R*,2R*)-3-(3-dimethylamino-1-ethyl-2-methyl-propyl)-phenol, (1R,2R,4S)-2-(dimethylamino)methyl-4-(p-fluorobenzyloxy)-1-(m-methoxyphen-yl)cyclohexanol or the stereoisomeric compounds thereof or the physiologically acceptable compounds thereof, in particular the hydrochlorides thereof, the derivatives thereof, such as esters or ethers, and processes for the production thereof are known, for example, from EP-A-693475 or EP-A-780369.

The formulations herein may also contain other active ingredients. These include, amongst others and for example, opioid antagonists (such as naloxone), aspirin, phenacetin, caffeine, acetaminophen, antihistamines, homatropine methylbromide, phenyltoloxamine citrate, barbiturates, or the like, or multiple combinations thereof.

Formulations herein may also comprise narcotic analgesics in combination with non-narcotic analgesics, antitussive preparations which contain narcotic or narcotic-like cough suppressants such as codeine, dihydrocodeinone, pholcodeine, and the like. Other products comprising a narcotic or narcotic-like composition for use as an antispasmodic in the gastrointestinal tract, such as Camphorated Opium Tincture, U.S.P., Opium Tincture, U.S.P., Opium extract, N.F., and the like.

Any desired amounts of the active substance may be used in the formulation described herein.

The term “ailment” is understood to be any physical or mental disorder or physical or mental disease; acute or chronic.

The term “maintenance dose” is referred to as the amount of active substance required to keep a desired mean steady-state concentration. For example, it is the amount of active substance administered to maintain a desired level of the substance in the blood.

The term “loading dose” is defined as a dose of active substance, often larger than subsequent doses, administered for the purpose of establishing a therapeutic level of the active substance.

The term “Eudragit E” is referred to as a pH dependent polymer and may be any dimethylaminoethyl methacrylate copolymers. Examples include, but are not limited to, Eudragit E™ and Eudragit E 100™.

The term “Eudragit RL” is referred to as a pH independent polymer and may be any poly(ethyl acrylate-co-methyl methacrylate-co-trimethylammonrroethyl methacrylate chloride. Examples include, but are not limited to, Eudragit RL™, Eudragit RL 100™, Eudragit™ RL PO, Eudragit™ RL 30 D, and Eudragit™ RL 12.5.

The terms “Eudragit NE”, “Eudragit RS” and “Eudragit NM” are referred to as pH independent polymers and may be any neutral copolymer based on ethyl acrylate and methyl methacrylate. Examples include, but are not limited to, Eudragit™ NE 30 D, Eudragit™ NE 40 D, and Eudragit™ NM 30 D, Eudragit™ RS 100, Eudragit™ RS PO, Eudragit™ RS 30 D, and Eudragit™ RS 12.5.

The terms “low”, “small” or “fine” particle size are interchangeable and refer to sizes lower than 1500 microns.

The terms “large”, “high” or “big” surface area with respect to surface area of the active ingredients or excipients as a population of particles, powder, crystals, granules etc. are interchangeable and refer to surface areas up to 10000 m²/g or higher.

The term “coat” may be variously characterized as a coating, layer, membrane, film, shell, capsule, or the like, and may substantially or completely surround or envelope.

The term “controlled release” may be variously characterized by “sustained release”, “sustained action”, “extended release”, “modified release”, “pulsed release”, “delayed release”, “targeted release”, “site specific release”, and “timed release”, which are used interchangeably in this application and are defined for purposes of the present invention as the time of release, the extent of release, the rate of release, the site of release and/or release of an active ingredient from a formulation at such a rate that when a dose of the active ingredient is administered in the sustained release, extended release, pulsed release, timed release, delayed release or controlled-release formulation, concentrations (levels) of the active ingredient are maintained within a desired range but below toxic levels over a selected period of time. In the case of in vivo administration, concentrations (levels) of the active ingredient could be measured in blood or plasma, for example. When administered in vivo the sustained release, extended release, pulsed release, timed release, delayed release or controlled-release formulation allows for a timely onset of action and useful plasma concentration of an active Ingredient to be maintained for longer than in the case of immediate-release forms.

The term “polymeric coating” or “polymeric coat” means any coating, which is formed from materials such as resins, pharmaceutical polymers or from materials formed by polymerization of one or more monomers to form linear or branched or cross-linked macromolecules.

The term “functional coating” as used herein is defined to mean a coating that affects the rate of release in-vitro or in-vivo of the active drug(s).

The term “non-functional coat” is defined to mean a coating that does not substantially affect the rate of release in-vitro or in-vivo of the active drug, but can enhance the chemical, biological, physical stability characteristics, or the physical appearance of the modified release dosage form.

The term “onset time” or “onset of action” represents latency, that is, the time required for the drug to reach minimum effective concentration or the time required for the drug to begin to elicit its action. It may also represent the time for complete release of the drug (e.g. loading dose). A “quick onset of action” represents a short period of time, for example, about 1 hour or less, for the drug to reach minimum effective concentration.

The terms “non-enteric polymer” and “pH independent polymer” are here understood to refer to a polymer which is non-enteric, i.e., which is not more soluble in non-acidic media than in acidic media. The terms “non-enteric polymer” and “pH independent polymer” therefore encompass polymers which are equally soluble in acidic, and neutral or basic media. The terms “non-enteric polymer” and “pH independent polymer” may additionally encompass polymers which are more soluble in acidic media than in neutral or basic media and/or swellable in non-acidic media.

The term “bittering agent” includes a compound used to impart a bitter taste, bitter flavor, etc.

The term “irritant” includes a compound used to impart an irritating or burning sensation.

### Oral Drug Delivery Formulations, Uses Thereof and Methods of Making Same

Oral drug delivery formulations, uses thereof and methods of making same are provided in order to reduce the potential for abuse, misuse or improper administration of an addictive substance or any active substance.

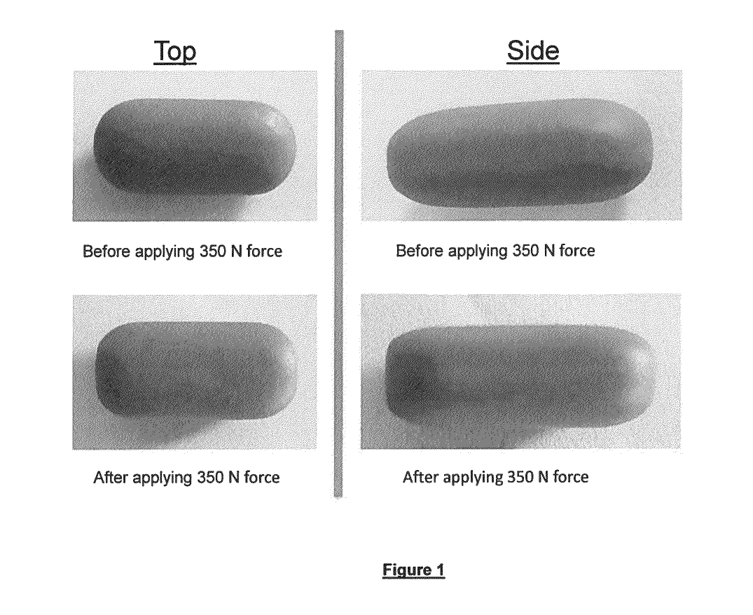

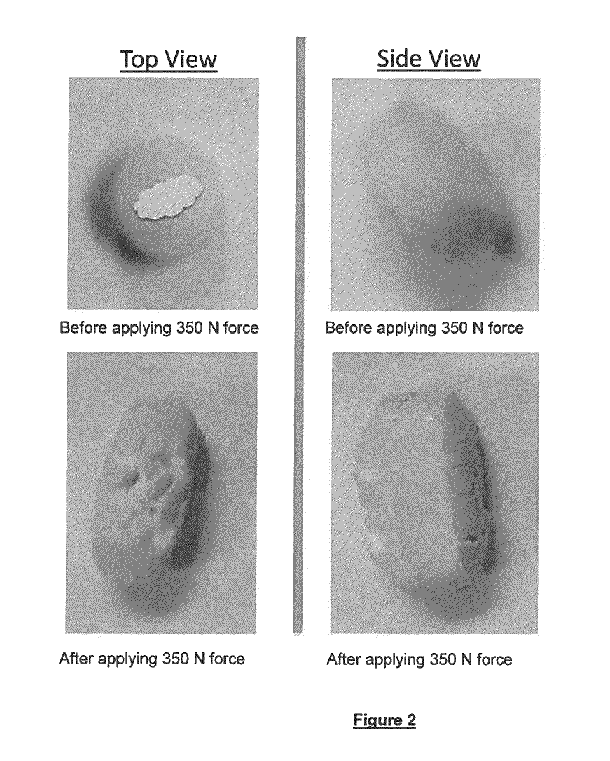

The formulations disclosed herein are reasonably resistant to perturbation or abuse/tampering and consequences thereof. FIGS. 1 and 2, for example, shows the effect of subjecting one embodiment of a tablet described herein to a 350 Newton force in comparison to the effect of subjecting a commercially available Oxycodone extended release tablet to the same force. The embodiment shows a higher breaking strength as compared to the commercially available Oxycodone tablet. In most embodiments, the formulations have a higher breaking strength as compared to conventional commercially available tablets, wherein the high strength is as a result of an external coat. The external coat makes the formulation resistant to perturbation. The external coat may be any suitable shape, including, but not limited to, cylindrical-like, cube-like, disc-like, a pod-like envelope or cocoon.

In general, the formulations provide the necessary amount of a drug to the patient over a period of time in order to accomplish the pharmaceutical effect (such as timely and adequate pain relief, inducing sleep, control of blood pressure and blood sugar levels, etc.), while decreasing or eliminating the problem of improper administration of medications and their use in a non-indicated or non-prescribed manner resulting in abuse, drug overdose, addiction, suboptimal efficacy or death.

In other aspects, the formulation comprises a loading dose having at least one active substance, wherein the release of the active substance shows a Point Of Divergence (POD), in a dissolution profile, with the loading dose representing a point in a timeline where the history of the dissolution or release rate changes from an onset of action to another set of points in the timeline represented by a controlled release.

### Perturbation or Tamper Deterrent Formulations

In certain embodiments, the formulation may reduce the potential for abuse of one or more active substances upon heating, microwaving, freezing and/or perturbation or disruption of the internal and external physical geometries of the formulation.

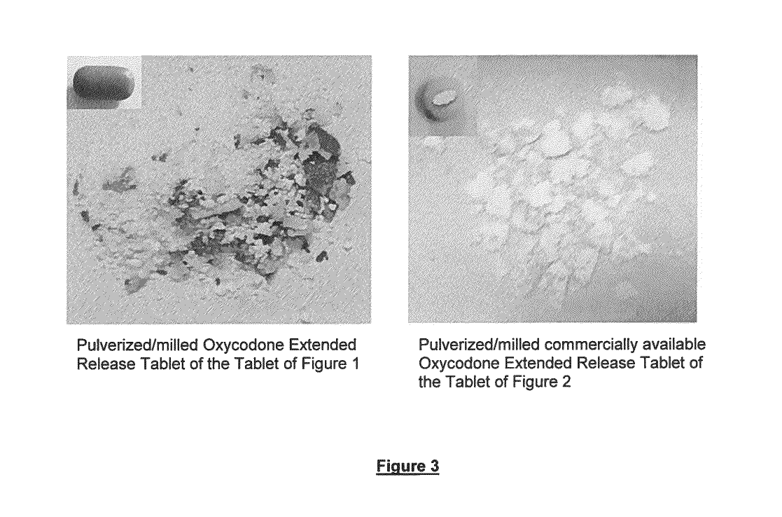

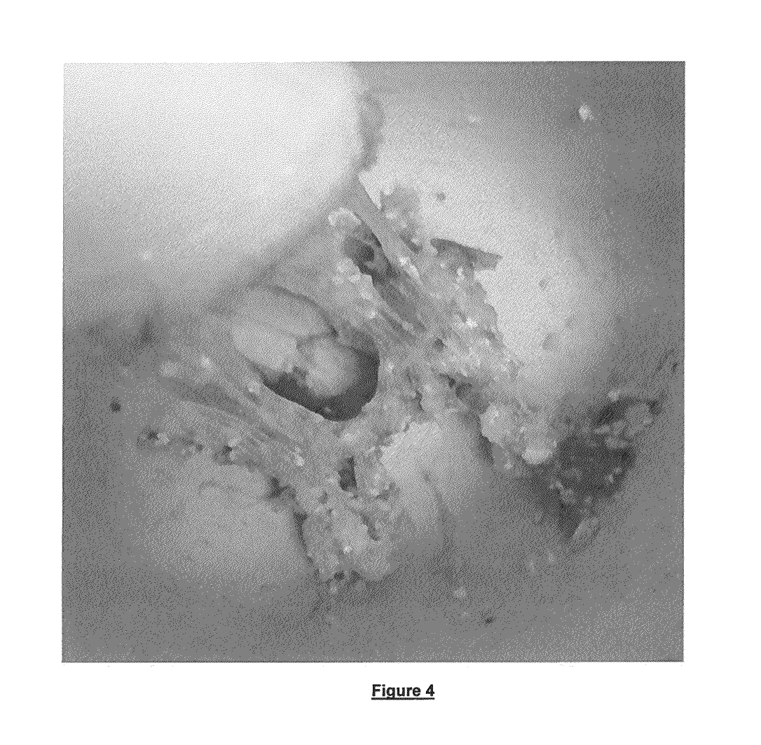

The perturbation or tamper deterrent formulation described herein may retard, or at least not increase, significantly, the instantaneous release or rate of release of the drug substance from a formulation when the physical integrity of the formulation containing the drug is compromised and the resulting formulation is subsequently snorted, injected, or swallowed. The composition is “physically compromised” when it is in a form other than an intact form. This can be achieved by various means such as by heating, microwaving, freezing, chewing, chopping, grinding, crushing, or placing into aqueous solvents, such as those containing an alcohol (e.g., ethyl alcohol), carbonated beverages and/or water itself. The formulation thus, can provide a deterrent to common methods of improper administration, including intravenous injection of the drug dissolved in solvent, and nasal or oral administration of the crushed formulation, as the drug will not be immediately and rapidly released from the formulation and in some cases the actual amount of drug release can be decreased as compared to an intact formulation. This is demonstrated in the example shown in FIGS. 3 and 4.

FIG. 3 shows a comparison of the pulverized formulation of Example 7 and the pulverized formulation of a commercially available formulation, wherein Example 7 is directed to a maintenance dose core, containing polyethylene oxide and Eudragit RL, which is then coated with a loading dose, followed by a coating of a pod-like envelope comprising Eudragit E. The pulverized formulation of Example 7, when combined with an aqueous solution, forms an agglomerated mixture as shown in FIG. 4 that prevents improper administration, including intravenous injection of the drug, and nasal or oral administration of the crushed formulation, as the drug will not be immediately and rapidly released from the formulation and, in this case, the actual amount of drug released is decreased as compared to an intact formulation. The combination of, for example, polyethylene oxide and Eudragit RL, in the formulation cause agglomeration when combined with an aqueous solution.

Certain formulations described herein may become objectionable and/or difficult to ingest, administer intravenously, and/or snort/inhale, for example, and as noted above with respect to Example 7, in view of the inclusion of excipients such as polyethylene oxide and Eudragit RL. In general, the formulation may have at least one excipient that comprises a swellable material, such as a pH independent polymer, that agglomerates in an aqueous solution such as, for example, an alcoholic and/or non-alcoholic beverage. The swellable material may be selected from swellable hydrophilic polymers such as cellulose polymers and their derivatives (such as for example, hydroxyethylcellulose, hydroxypropylcellulose, carboxymethylcellulose, hydroxypropylmethylcelulose, and microcrystaline cellulose), polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly(2-ethyl-2-oxazoline), poly(ethyleneimine), polyurethane hydrogels, gums, alginates, lectins, carbopol and combinations thereof. Other specfic examples include carbomers, polyethylene oxide or hydrophilic polymers that are lightly cross-linked, such cross-links being formed by covalent or ionic bond, which interact with water and aqueous biological fluids and swell or expand to some equilibrium state. The amount of swellable material may range from about 15 wt % to about 90 wt % of the core or layer/coat or from about 40 wt % to about 90 wt % of the core or layer/coat, typically, from about 50 wt % to about 80 wt % or from about 70 wt % to about 80 wt %. Combinations of swellable materials may be used. In particular, combinations of at least one pH independent Eudragit polymer and another swellable polymer. The Eudragit polymer may range from about 1 wt % to about 30 wt % of the core or layer/coat, typically, from about 1 wt % to about 10 wt % or from about 3 wt % to about 6 wt %. Examples of the Eudragit polymers used are Eudragit RL, Eudragit NE, Eudragit RS and Eudragit NM. Specific examples include, but are not to be limited to, Eudragit RL™, Eudragit RL 100™, EUDRAGIT™ RL PO, EUDRAGIT™ RL 30 D, EUDRAGIT™ RL 12.5, EUDRAGIT™ NE 30 D, EUDRAGIT™ NE 40 D, and EUDRAGIT™ NM 30 D, EUDRAGIT™ RS 100 EUDRAGIT RS PO, EUDRAGIT™ RS 30 D, and EUDRAGIT™ RS 12.5. Such formulations may be objectionable to ingest, administer intravenously, and/or short/inhale on perturbation, pulverizing, crushing, grinding, milling, cutting or chewing into sizes that may range from very fine to coarse particles, granules or spheres. Such embodiments are not objectionable to ingest when taken intact.

The formulation may have at least one excipient that comprises a swellable material, which includes pH independent polymer(s), in any component of the formulation, however, typically, they are included in at least one of the drug coat, drug layer, or drug core.

Therefore, in certain embodiments, the formulations are formed such that an addictive substance comprised therein is not easily soluble and immediately available upon crushing and attempting to dissolve it for intravenous injection or to obtain access to the total drug immediately upon oral ingestion of the crushed formulation. In general, a formulation may comprise at least one active substance; and at least one excipient, wherein dissolution of the pulverized/milled formulation in alcoholic and/or non-alcoholic beverages causes the formulation to agglomerate.

The formulations described herein can be more objectionable and/or resistant to perturbation and consequences thereof, when presented as an intact dosage form and/or when heated, burned, microwaved, frozen, perturbed, pulverized or crushed or ground or milled or cut into one or more sizes ranging from very fine to coarse particles, granules or spheres. The formulations described herein, with or without a loading dose, may demonstrate non or insignificant loss in controlled release properties or does not result in instantaneous or rapid release of active content when heated, burned, microwaved, frozen, perturbed, pulverized or crushed or ground or milled or cut into one or more sizes ranging from very fine to coarse particles, granules or spheres as compared to when presented as an intact dosage form.

For example, the formulation comprises a loading dose. The formulation, which in spite of the presence of a loading dose, is more difficult to be subdivided, crushed or abused via crushing to instantaneously or significantly release its active ingredient as compared to known commercial products. The formulation may also comprise a loading dose, which assures a quick onset of action and a sustained action via timely release of the loading dose and adequately controlled maintenance dose thereby providing effective pain relief as compared to known commercial products. These types of formulations may resist abuse or unintended misuse without compromising therapeutic effectiveness. A formulation having a loading dose, as used herein, represents an active substance that is released at a higher rate in comparison to another dose of the active substance in the same formulation. In other words, in the formulation, a greater amount of the active substance is released in a certain time interval in comparison to, for example, another dose of the active substance in the formulation that is released in a similar time interval. For example, in an embodiment, 50% by weight of the active substance in the formulation is released in 1 hour; however, in the following hour, only 10% by weight of the active substance is released.

In embodiments, the loading dose releases the active substance more than one times the rate of release of any subsequent dose of active substance. The loading dose establishes a therapeutic level of the active substance in a short time interval. Such formulations defined herein, permit therapeutic treatment without the potential for abuse thereof.

In a specific embodiment, the formula comprises i) at least one active substance, wherein release of the active substance, onset of action, is potentiated by the presence of a loading dose of the active substance and ii) at least one coating for controlling the release of the loading dose, wherein at least one of the coating(s) comprises Eudragit E(dimethylaminoethyl methacrylate copolymer) and, optionally, excludes any active substance. In another embodiment, the amount of Eudragit E in the coat makes the formulation more difficult to be inadvertently subdivided, crushed or abused via crushing to instantaneously or significantly release its active ingredient as compared to known commercial products. In a further embodiment, the formulation comprises at least one primary active substance and at least one coat that comprises Eudragit E, wherein the formulation is free of any active substance external to the coat.

In certain embodiments, when the formulation is at least one of perturbed, pulverized or crushed or ground or milled or cut into one or more sizes ranging from very fine to coarse particles, granules or spheres and mixed with from about 10 ml to about 1000 ml aqueous solution will not lead to at least one of solubilisation, significant, rapid and/or instantaneous drug release and/or release of most of the drug in about the first hour. In other embodiments, even if the mixture was mixed under low to moderate to high agitation or speed of mixing will not lead to at least one of solubilisation, significant, rapid and/or instantaneous drug release and/or release of most of the drug in about the first hour.

The aqueous solution may be any suitable aqueous beverage for consumption such as alcoholic and/or non-alcoholic beverages.

In other embodiments, the formulation having a loading dose which when the formulation is at least one of perturbed, pulverized or crushed or ground or milled or cut into one or more sizes ranging from very fine to coarse particles, granules or spheres and mixed with from about 1 ml to about 1000 ml aqueous solution under low to moderate to high agitation or speed of mixing, only from about 1 to about 30% of the loading dose is extracted and/or released in 10 minutes.

In another embodiment, the formulation having a loading dose which when the formulation is at least one of perturbed, pulverized or crushed or ground or milled or cut into one or more sizes ranging from very fine to coarse particles, granules or spheres and mixed with from about 10 ml to about 1000 ml aqueous solution under low to moderate to high agitation or speed of mixing, only from about 1 to about 30% of the loading dose is extracted and/or released in about 10 minutes to about 60 minutes; typically about 10, about 20, about 30 or about 60 minutes.

In a further embodiment, the formulation when perturbed, pulverized or crushed or ground or milled or cut into one or more sizes ranging from very fine to coarse particles, granules or spheres and mixed with from about greater than about 10 ml to about 1000 ml alcoholic or non-alcoholic beverages under low to moderate to high agitation or speed of mixing only from about 1 to about 30% is extracted and/or released in about 10 minutes to about 60 minutes, typically about 10, about 20, about 30 or about 60 minutes.

In another aspect, the formulation when perturbed, pulverized or crushed or ground or milled or cut into one or more sizes ranging from very fine to coarse particles, granules or spheres and mixed with from about greater than about 10 ml to about 1000 ml aqueous solution under low to moderate to high agitation or speed of mixing only from about 1 to about 40% is extracted and/or released in about 2 to about 5 hours. The aqueous solution may be alcoholic or non-alcoholic beverages.

In another embodiment, the formulation comprises one or more active ingredients with a particle size less than about 2000 microns and materials selected from release retarding agents, gelling agents, polymers, co-polymers, cross-linked polymers and non-cross linked polymers in an amount and ratio which is sufficient to prevent the compromising or significant and or complete loss of integrity of the controlled release mechanism of the composition when burned, microwaved, heated, frozen, perturbed, pulverized or crushed or grind or milled or cut into one or more sizes ranging from very fine to coarse particles, granules or spheres and placed in contact with from about 10 ml to about 1000 ml of gastrointestinal (GI) fluids, simulated GI fluids, aqueous solution, alcoholic or non-alcoholic beverages.

Accordingly, in certain embodiments, there is provided a formulation with a loading dose that can be effectively employed to reduce the problems of dose dumping of one or more active substances. The formulation may have a loading dose that can be effectively employed to reduce the potential for abuse of one or more active substances upon heating, microwaving, freezing and/or perturbation or disruption of the internal and external physical geometries of a delivery formulation.

In another embodiment, a formulation has active pharmaceutical ingredient(s) and/or inactive ingredient(s) having a large surface area that can be effectively employed to control the release of one or more active substances in a formulation or prevent the instantaneous release of most of the dose in the formulations upon perturbation or disruption of the internal and external physical geometries of the said formulation of composition. Examples of perturbation or disruption can include heating, microwaving, and/or freezing.

Formulations with active pharmaceutical ingredient(s) and/or inactive Ingredient(s) having a large surface area can be effectively employed to reduce the problems of dose dumping of one or more active substance.

### pH Dependent, Ion Exchange Dependent or Intestinal Bacterial Flora or Enzymes Dependent Formulations

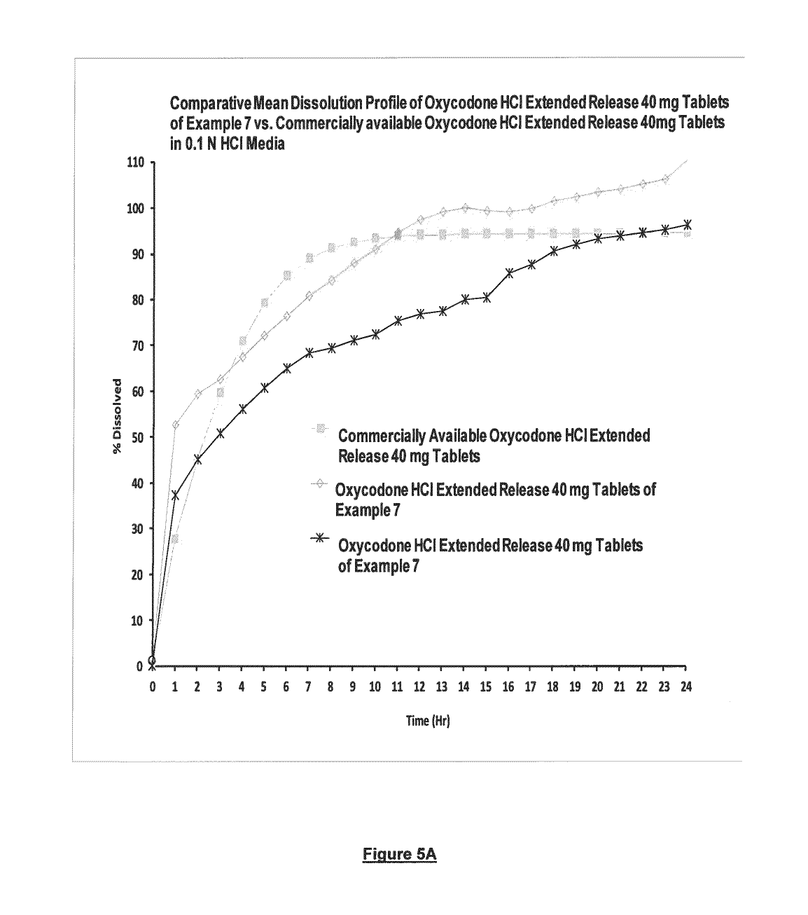

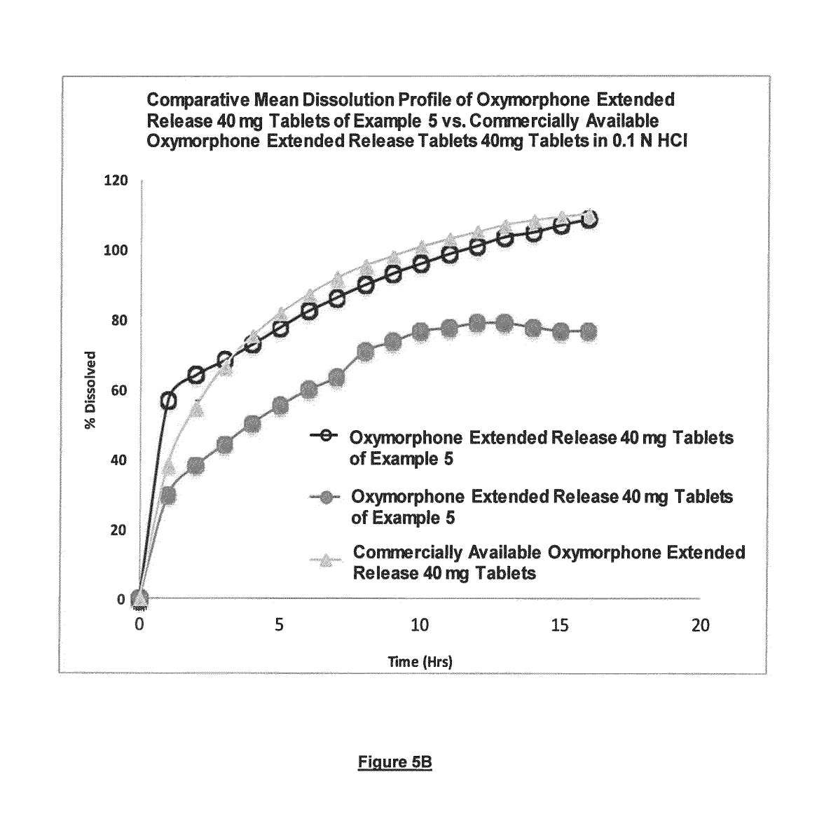

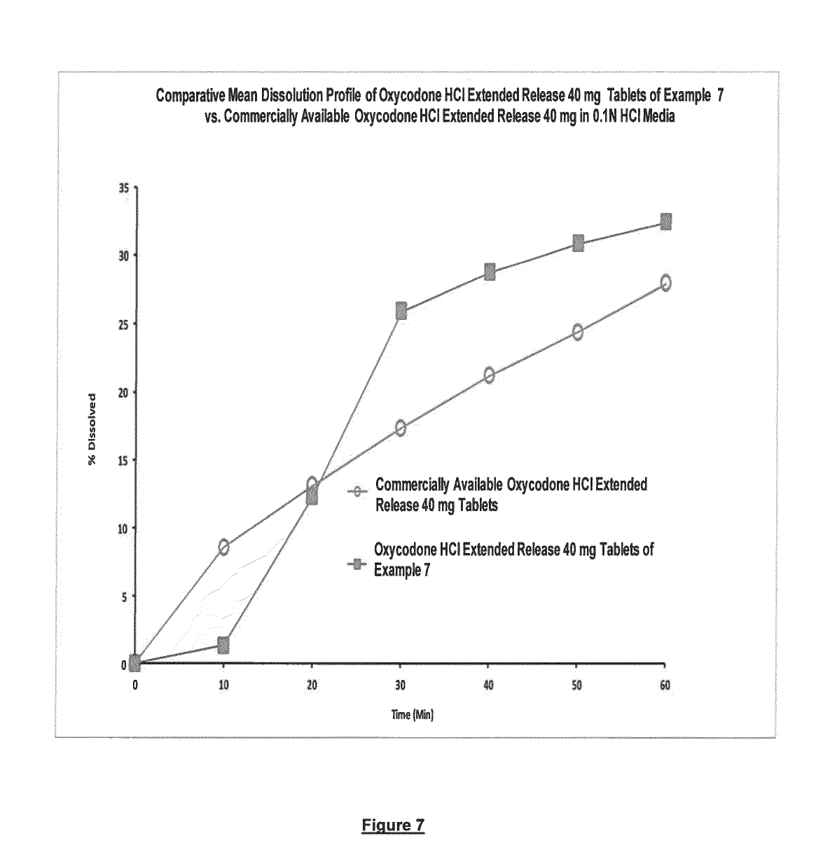

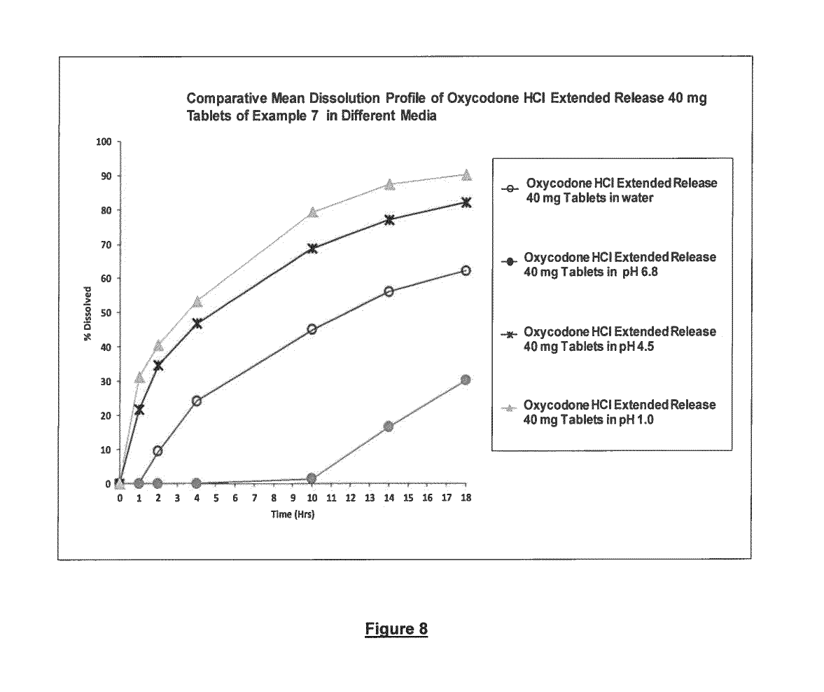

In embodiments, the formulations contain a loading dose surrounded by one or more coats in which drug release, onset of action, sustained action and effectiveness is potentiated by the presence of a loading dose, which is triggered when the coat(s) is activated by a pH dependent mechanism, an ion exchange mechanism or intestinal bacterial flora or enzymes. Examples are shown in FIGS. 5, 7, and 8 which show pH dependency on dose dumping, wherein the formulations (Examples 5 and 7) are directed to a maintenance dose core, which is then coated with a loading dose, followed by a coating of a pod-like envelope comprising Eudragit E.

The rate and extent of release of a loading dose in such embodiments is ion exchange dependent, dependent on intestinal bacterial flora, dependent on intestinal enzymes, and/or pH dependent. For example, the formulation, which in the presence of gastric fluid up to a pH of about 5.0 will release a loading dose having a greater magnitude than in gastric fluid of a pH above about 5.0

The formulation, in which the presence of gastric fluid or aqueous media that is less acidic to alkaline pH, trigger the release of a lesser amount of a loading dose. An optimum amount of a loading dose is released in the presence of acidic liquid media. In typical embodiments, the amount of active ingredient(s) is released in the first hour or in less than four hours from time zero of a release cycle or profile, in-vitro and/or in-vivo.

The formulations may be directed to a dosage form containing a matrix or non-matrix core incorporating one or more active ingredients, excipients, and release controlling agent(s). The core may be surrounded by a coat of one or more active ingredients, followed by another coat which is soluble in liquid media with a pH less than about 5.0 but insoluble in a pH above about 5.0 wherein the dosage form does not result in instantaneous or rapid release of active content when heated, burned, microwaved, frozen, perturbed, pulverized or crushed or ground or milled or cut into one or more sizes ranging from very fine to coarse particles, granules or spheres as compared to when presented as an intact dosage.

In certain embodiments, the formulation has active pharmaceutical ingredient(s) and/or excipients and/or release controlling agents with low particle size and higher surface area. The formulation is capable of higher rates of release in liquid media with a pH of less than about 5.0 than in liquid media with a pH above about 5.0.

The formulation has one or more release cycles. The rate of release of the loading dose is higher than the rate of release of the maintenance doses. In typical embodiments, the formulation, which on perturbation or pulverizing or crushing or grinding or milling or cutting or chewing into one or more sizes ranging from very fine to coarse particles, granules or spheres will not result in the dose dumping of the active ingredient or instantaneous release of all of its dose.

In other embodiments, the formulation has one or more active substances in a pharmaceutically effective amount, wherein the formulation is configured such that when the intact formulation is brought into contact with a solution having a pH below 5, not more than about 55% of the amount of drug is released in 1 hour and not less than about 30% is released in pH 1-2 in 1 hour. The rate of drug release is lower at a pH greater than about 5 than at a pH lower than about 5. In other embodiments, the formulation has a higher rate of release and/or higher loading dose released in acidic media than in basic media.

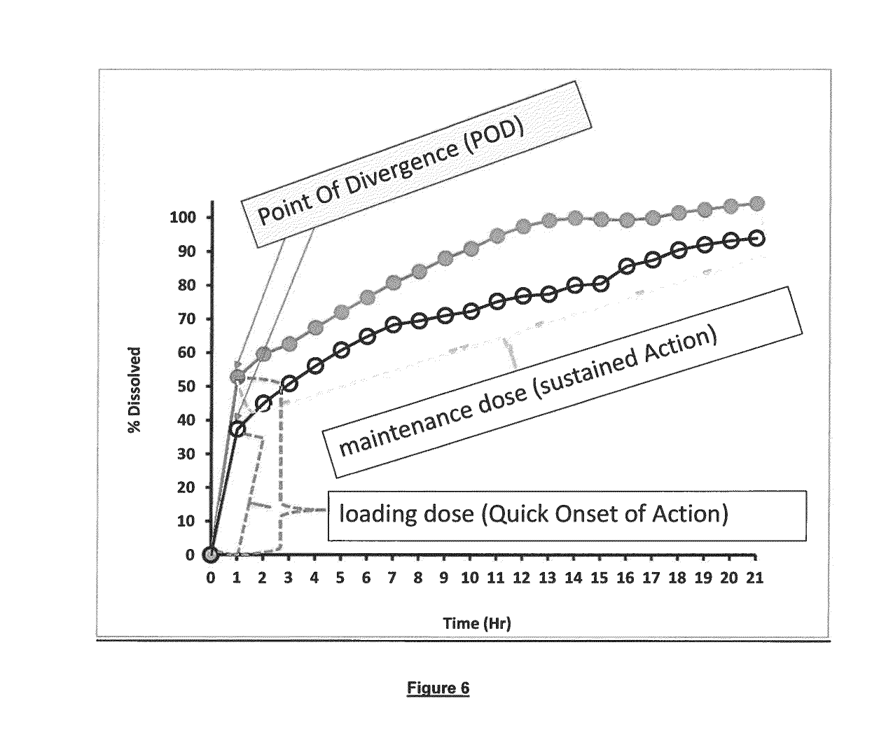

In certain embodiments of the formulations, drug release, onset of action, sustained action and effectiveness is potentiated by the presence of a loading dose, which is triggered or activated by a pH dependent mechanism, ion exchange dependent mechanism or intestinal bacterial flora/enzymes dependent mechanism or a combination thereof. Drug release shows a clearly defined Point Of Divergence (POD), in the dissolution profile or drug release time lines, with the loading dose representing a point in a timeline where the history of the dissolution or drug release rate begins to change from a quick onset of action to another set of points in the timeline represented by a sustained action and provision of a maintenance dose. See, for example, FIG. 6. Prior to the POD, there is no history of a timeline of sustained action. Certain formulations can have a sustained action followed by a quick onset of action.

In the various embodiments described throughout the description, the formulations may have a loading dose, for example, the formula comprises i) at least one active substance, wherein release of the active substance, onset of action, is potentiated by the presence of a loading dose of the active substance and ii) at least one coating for controlling the release of the loading dose, wherein at least one of the coating(s) comprises Eudragit E, which, optionally, excludes any active substance. In a further embodiment, the formulation comprises at least one primary active substance and at least one coat that comprises Eudragit E, wherein the formulation is free of any active substance external to the coat. The surface area coverage by Eudragit E in the coat is at least about 5 mg/cm², more typically, at least about 10 mg/cm², and even more typically, at least about 20 mg/cm². For example, the Eudragit E may be present in an amount of from about 5 mg/cm² to about 100 mg/cm²; typically, about 10 mg/cm² to about 100 mg/cm² and even more typically, about 10 mg/cm² to about 100 mg/cm². The amount of Eudragit E in the coat may be from about 5 wt % to about 80 wt % of said at least one of said at least one coating, typically, about 30 wt % to about 60 wt %, or more typically, 40 wt % to about 60 wt %. The Eudragit E is used in such an amount to prevent dose dumping of the active ingredient(s), typically, in alcohol. In contrast, the formulations may not have a loading dose. Instead, the formulation may comprise at least one active ingredient as a controlled release dose, for example, and coating(s), whereby Eudragit E may be part of at least one of the coats. The Eudragit E may have similar surface area coverage as described above. In an embodiment, the formulation may be directed to a dosage form containing a matrix or non-matrix core optionally incorporating one or more active ingredients, excipients, and release controlling agent(s). The core may be surrounded by a coat of one or more active ingredients, followed by another coat that includes Eudragit E, in which the active ingredient(s) is released when the Eudragit E coat is activated by a pH dependent mechanism, an ion exchange mechanism or intestinal bacterial flora or enzymes.

Formulations in the Presence of Alcoholic and/or Non-Alcoholic Beverages

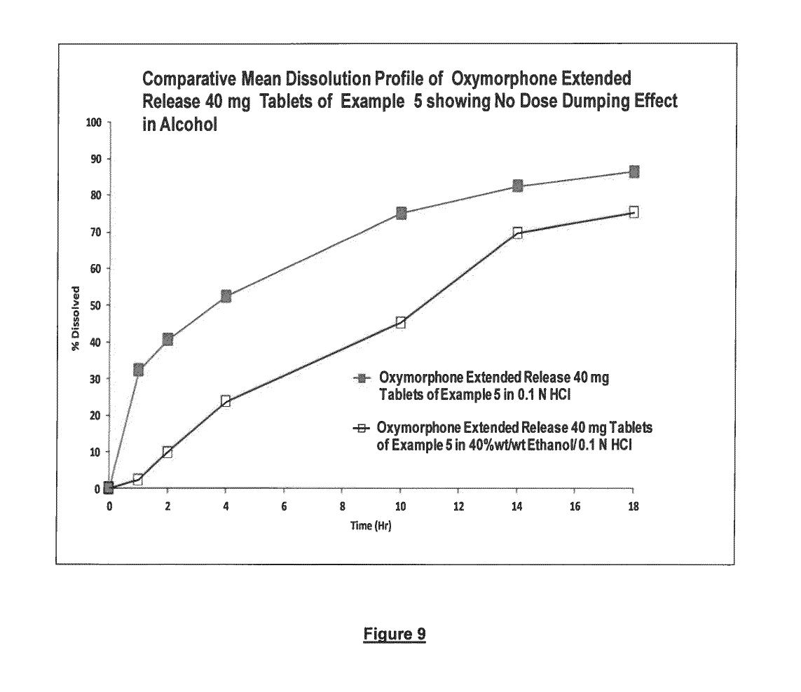

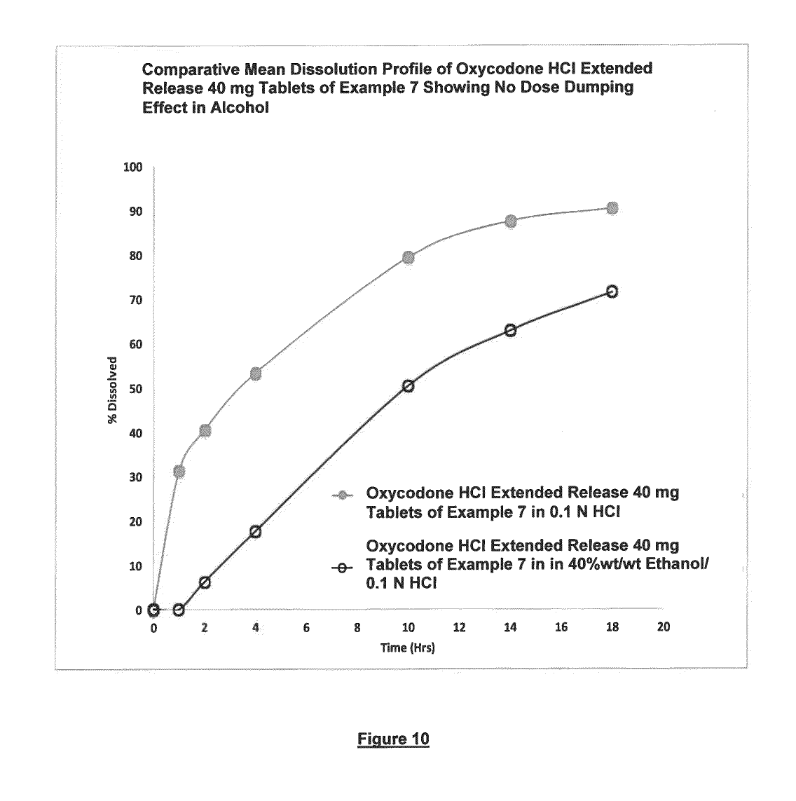

Certain formulations, in the presence of alcohol, release similar or lesser amounts at a similar or lesser rate when compared to being in the presence of acidic or aqueous solutions. See for example, FIGS. 9 and 10 which show less or no dose dumping in the presence of alcohol, wherein the formulations (Examples 5 and 7) are directed to a maintenance dose core, which is then coated with a loading dose, followed by a coating of a pod-like envelope comprising Eudragit E.

The formulation may have perturbation or tamper resistant properties, prevent the instantaneous release of the active ingredient(s) upon heating/evaporation, microwaving, freezing and/or or upon perturbation, pulverizing, crushing, grinding, milling or cutting them into one or more sizes ranging from very fine to coarse particles, granules or spheres and there is less or no dose dumping of one or more active substances in the presence of alcohol.

In certain embodiments, and as shown in Example 42, less than about 30% by weight of the dose is released as a vapor for inhalation when the formulation is subjected to heat. In other embodiments, even less than about 10% by weight of the dose is released as a vapor. The formulation may be milled prior to heating and the heating can be achieved, for example, with an open flame or other heat source. Temperatures may be less than or equal to about 540° C.

The formulation can comprise an active substance in a pharmaceutically effective amount, wherein the formulation is configured such that when it is contacted with an alcohol or consumed with an alcoholic beverage, the rate of active substance is released from the formulation within a time period selected from the group consisting of about 0.5 hours, about 1 hour, about 2 hours, about 4 hours and about 8 hours and the release is substantially the same or lower, typically less than about 40%, more typically less than about 30%, and most typically less than about 20%, than the rate of drug released when the formulation is administered with a non-alcoholic solution.

The formulation can comprise an active substance in a pharmaceutically effective amount wherein the formulation is configured such that when it is contacted with an alcohol or consumed with an alcoholic beverage, there is a lag time whereby the active substance is released in at least about 1 hour.

In another aspect, the formulation provides adequate and timely drug release and yet is less amenable to the effects of perturbation, tampering, and/or abuse and does not significantly dose dump in the presence of alcohol.

In embodiments, the formulation has a loading dose of active substance(s) and inactive substance(s) with a high surface area.

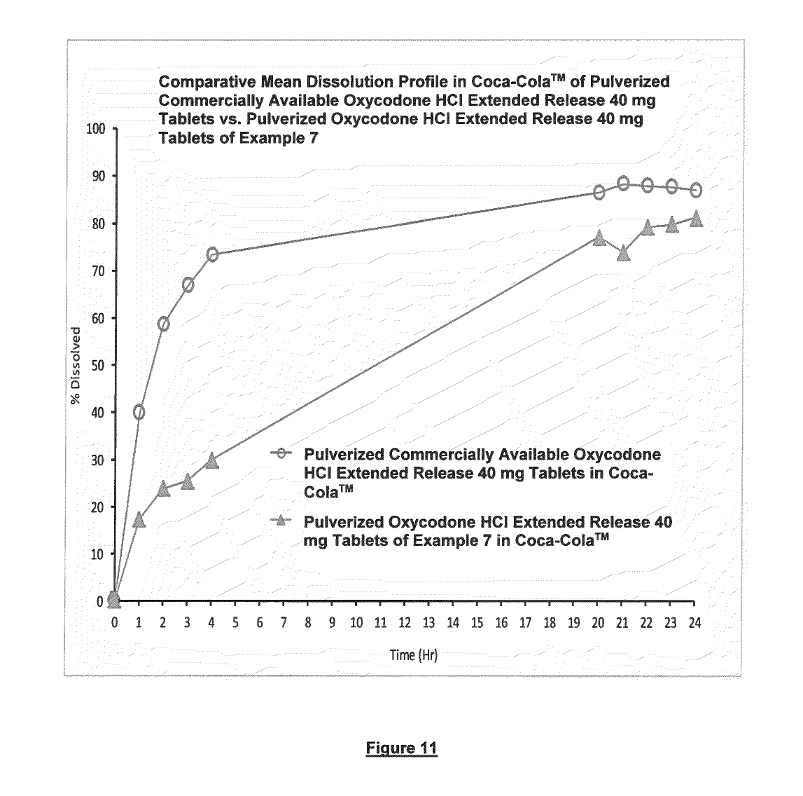

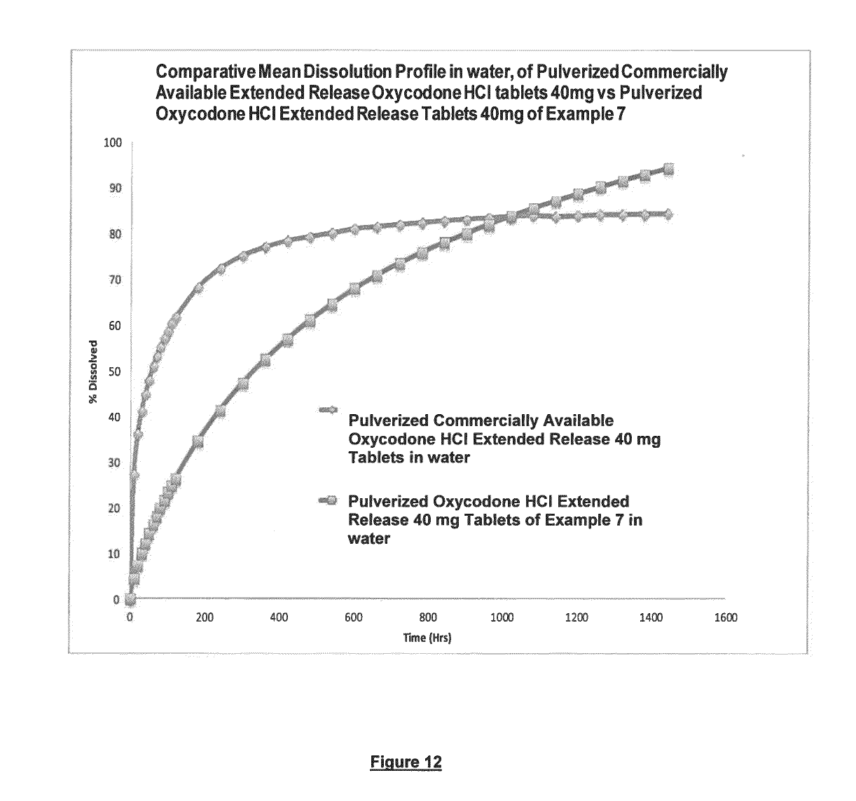

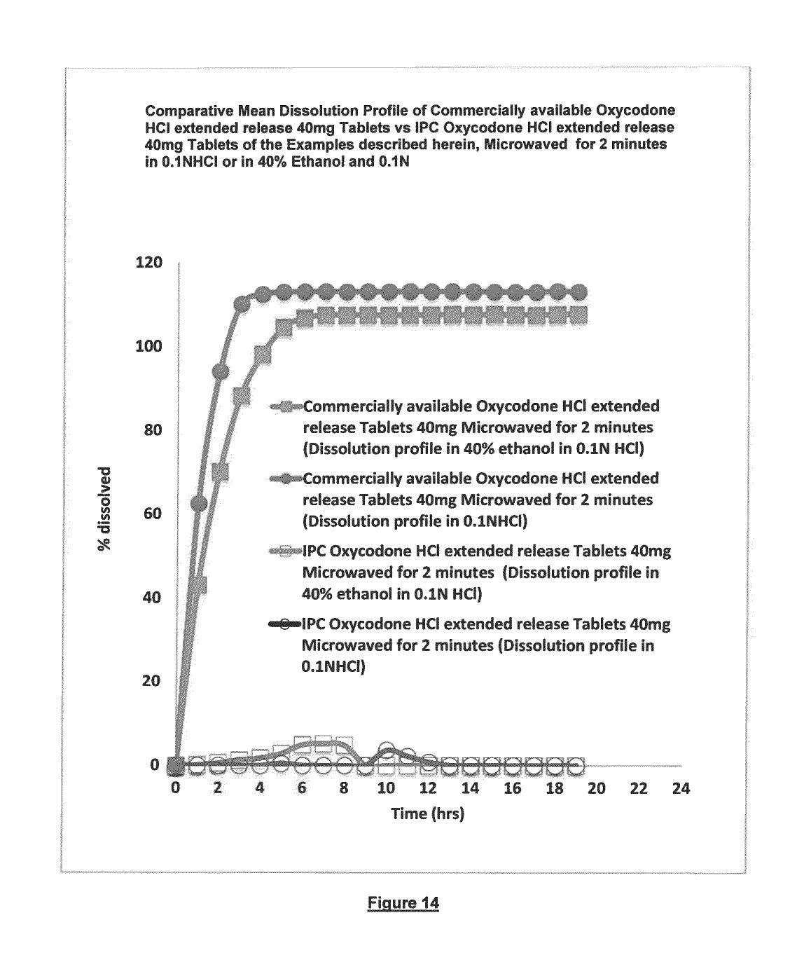

In another embodiment, there is provided a controlled release formulation with a loading dose that is resistant to being easily sub-divided, resistant to abuse, and/or resistant to tampering. The loading dose of the formulation is shielded from dose dumping in alcohol or common beverages used by addicts and/or inadvertent instantaneous release of significant or all of the active ingredient when microwaved, burned, heated, perturbed, pulverized or crushed or ground or milled or cut into one or more sizes ranging from very fine to coarse particles, granules or spheres. See, for example, FIGS. 11 and 12, wherein the formulation (Example 7) is directed to a maintenance dose core, which is then coated with a loading dose, followed by a coating of a pod-like envelope comprising Eudragit E. See also FIG. 14, which shows a mean dissolution profile of Oxycodone tablets of the Examples described herein compared to the commercially available Oxycodone HCl extended release tablets, microwaved for 2 minutes in 0.1N HCl or in 40% Ethanol and 0.1N HCl, which indicates that the dissolution is not perturbed by microwaving. In certain embodiments, less than about 20% of the dose in the formulation is released after microwaving for about 2 minutes and thereafter, exposing the microwaved formulation to aqueous media.

In accordance with yet another aspect, a modified release, delayed release, controlled release or extended release formulation and method in which the physicochemical nature of the composition helps to prevent significant dose dumping in the presence of alcohol and also discourage abuse.

A formulation incorporating an active substance with small particle size and large surface area and the choice of a suitable polymer makes it harder for dose dumping of an addictive substance in the presence of alcohol or during co-ingestion of alcohol.

The formulations may comprise any active ingredient, especially medications that are subject to abuse due to the presence of active ingredients that can produce an emotional, psychological, euphoric, depressive or generally psychedelic experience. More specifically, it may pertain to medicaments whose unintended or improper administration may lead to abuse, drug overdose, suboptimal efficacy or death; medicaments used to manage pain, medicaments used to reduce or eliminate anxiety attack (psychotherapeutic drugs), medicaments that are used as stimulants and sleeping pills, cardiovascular agents, antidiabetics, acid labile drugs and in general medicaments whose intended effects may be compromised if the intact dosage form is heated, burned, microwaved, frozen, perturbed, pulverized or crushed or ground or milled or cut into one or more sizes ranging from very fine to coarse particles, granules or spheres.

Formulations Objectionable to Chewing, Sucking, Licking and/or Holding in the Mouth

A bittering agent may optionally be present in the formulations to make the compromised formulation objectionable to chewing, sucking, licking and/or holding in the mouth. The pharmaceutically acceptable bittering agents used may be denatonium benzoate, denatonium, saccharide esters such as saccharide Sucrose octaacetate, naringin, phenylglucopyranose, benzyl glucopyranose, tetramethylglucose and glucose pentaacetate, or quassin. The most typical is Sucrose octaacetate. With the inclusion of a bittering agent in a formulation, when the formulation is tampered with, the bittering agent imparts a discomforting quality to the abuser to typically discourage the inhalation or oral administration of the tampered formulation, and typically to prevent the abuse of the formulation.

Suitable bittering compositions may include bittering agents or analogues thereof in a concentration 20 to 1000 ppm, typically 10 to 500 ppm and most typically 5 to 100 ppm in the finished product.

In an embodiment, the formulation comprises a core containing one or more active substance(s) with or without a bittering agent, surrounded by a film optionally containing one or more active substance(s) embedded in a functional or non-functional coat or coat matrix and further surrounded by a functional or non-functional coat or coat matrix. The coat or coat matrix can be applied by spraying or dry coating or encapsulation or by a combination of these methods.

In certain embodiments, the formulation is objectionable to chewing, sucking, licking and/or holding in the mouth for more than about 1 minute; for more than about 5 minutes, or for more than about 10 minutes. In another embodiment, the formulation is objectionable to chewing, sucking, licking and/or holding in the mouth for less than about 10 minutes but greater than about 30 seconds. Moreover, in similar embodiments, the formulation will not permit release or will not release a significant amount of the active ingredient(s) in the pH environment of the mouth.

An irritant may be present in the formulations. With the inclusion of an irritant (e.g., capsaicin) in the formulation, when the formulation is tampered with, the capsaicin imparts a burning or discomforting quality to the abuser to typically discourage the inhalation, injection, or oral administration of the tampered formulation, and typically to prevent the abuse of the formulation. Suitable capsaicin compositions include capsaicin (trans 8-methyl-N-vanillyl-6-noneamide) or analogues thereof in a concentration between about 0.00125% and 50% by weight, typically between about 1 and about 7.5% by weight, and most typically, between about 1 and about 5% by weight of the formulation but not more than 50 mg/kg body weight daily intake.

### Administration

The formulation may be administered in-vivo oral, vaginal, anal, ocular, subcutaneous, intramuscular administration or for implantation. The composition may also be used for in vitro or ex vivo delivery of an addictive substance. It may be targeted at specific sites in the gastrointestinal tract or to specific organs. It may be applied occularly and transdermally in a pouch or patch. It is evident that the physical state of the formulation and the particular method of application may vary accordingly.

The formulation may reduce the potential for improper administration or use of drugs but which, when administered as directed, is capable of delivering a therapeutically effective dose. In particular, the formulation addresses the need for a drug product, which, compared to conventional formulations, decreases the intensity, quality, frequency and rate of occurrence of the “euphoria” and other untoward effect, which can occur with improper administration.

In yet another embodiment, the formulation, despite the presence of a loading dose, reduces the potential for improper administration or use of drugs but which, when administered as directed, is capable of delivering in a timely fashion, a therapeutically effective dose. In particular, the formulation addresses the need for a drug product, which, compared to conventional formulations, decreases the intensity, quality, frequency and rate of occurrence of the “euphoria” and other untoward effect, which can occur with improper administration.

### Various Formulations

In one embodiment, the formulation comprises: one or more of a modified release, delayed release, controlled release and/or extended release drug core referred to as the maintenance dose; surrounded first by one or more layers of active substance(s) embedded in a non-functional coat; followed by one or more layers of functional coat.

In certain embodiments, some or all of the loading dose of the formulation is incorporated into and/or onto the core of the formulation. In other embodiments, the loading dose is separated from the maintenance dose; the loading dose is only incorporated in the maintenance dose; or the maintenance dose is only present in the core. The formulation may have the loading dose incorporated in the maintenance dose in addition to a separate loading dose external to the maintenance dose.

The formulation may have one or more loading doses.

In a further embodiment, the formulation, which when taken intact as intended, has a core and one or more active substance(s) layers internal or external to the core which may contribute to the loading dose. The formulation, which when taken intact as intended, the core may contribute to the maintenance dose and one or more active substance(s) layers internal or external to the core contributes to the loading dose.

In the various formulations, the loading dose is released in one or more time intervals.

The formulation may comprise one or more active substance(s) in a pharmaceutically effective amount, wherein the formulation is configured such that when the formulation is administered in physically compromised form to a subject, the rate of active substance(s) released from the composition, within a time period selected from the group consisting of about 0.5 hours, about 1 hour, about 2 hours, about 4 hours and about 8 hours, is substantially the same or lower than the rate of active substance(s) released when the formulation is administered in an intact form.

The formulation may comprise one or more active substance(s) in a pharmaceutically effective amount, wherein the formulation is configured such that when the formulation is administered in physically compromised form to a subject, the rate of active substance(s) released from the composition, within a time period selected from the group consisting of about 0.5 hours, about 1 hour, about 2 hours, about 4 hours and about 8 hours, is substantially the same or lower, typically less than 20%, more typically less than 30%, and most typically less than 40%, than the amount of active substance(s) released when the pharmaceutical composition is administered in an intact form.

The formulation may comprise one or more active substance(s) in a pharmaceutically effective amount, wherein the formulation is configured such that when the formulation is administered in an intact form, at least 50% of the amount of active substance(s) is released after about 8 hours and when the formulation is administered in a physically compromised form at most about 55%, typically at most about 50%, more typically at most about 30%, of the amount of active substance(s) is released in about 1 hour.

The formulation may comprise one or more active substance(s) in a pharmaceutically effective amount, wherein the formulation is configured such that when the formulation is administered in an intact form, at least 80% of the amount of active substance(s) is released after about 1 hour and when the formulation is administered in a physically compromised form at most about 70% of the amount of active substance(s) is released in about 1 hour.

In yet another embodiment, the formulation is designed such that in the treatment of severe to moderate pain using opioid analgesics timely delivery of onset of pain relief and adequate pain relief is experienced by the patient from about 30, about 60, about 120, about 180 or about 240 minutes. In another embodiment, the formulation is designed such that the formulation or composition can be administered every 8 hours to 12 hours to every 24 hours.

In certain formulations, the active substance(s) and/or inactive substance(s) used in the formulation have a fine, small or low particle size and large, high or big surface area. Accordingly, the particle size is less than 1500 Microns, typically less than 1000 microns and more typically less than 400 microns.

In certain formulations, the loading dose is applied as a coat around the core of the formulation or composition.

The formulation may have one or more of an immediate release, modified release, delayed release, controlled release or extended release drug core; surrounded first by one or more layers of drug embedded in a non-functional coat followed by one or more layers of functional coat. The active substance may be, without limitation, an opioid agonist, a narcotic analgesic, barbiturates, central nervous system stimulants, tranquilizers, antihypertensive, antidiabetics, and/or antiepileptics. Prior to incorporation within the core or coat, the active substance may be in any suitable form known in the art, liquid, semi-solid or solid, and may be homogenously or non-homogenously dispersed in the core.

The formulation can be a solid unit formulation such as, and without being limited thereto, a tablet, granules, spheres, particles, beads, capsules or microcapsules. In another embodiment, the formulation optionally has a loading dose surrounded by a protecting coat that has a high surface area and is pH dependent, ion-exchange dependent, and/or bacterial flora/enzyme dependent.

It will be understood that the formulations may not be limited to addictive substances, and may also be useful in formulations of any active ingredient or substance.

Several embodiments of the formulations are provided:

In an embodiment, there is provided a formulation that is effectively employed to control the release of one or more active substances or prevent the instantaneous release of the entire dose in the formulation upon perturbation or disruption of the intemal and/or external physical geometries of the formulation.

In a further aspect, the loading dose of the formulation is incorporated into at least one of the 1) core, 2) external to the core and 3) both 1 and 2.

The formulation may have a modified release, delayed release, controlled release or extended release formulation and in which the physicochemical nature of the formulation is used to reduce the potential and consequences (drug overdose, addiction, suboptimal efficacy, and/or death) of improper administration of medications and their use in a non-indicated or non-prescribed manner.

In one embodiment, a formulation comprises: i) a core comprising one or more active substances in a matrix and ii) substantially surrounded by a coat comprising one or more of the same or different active substances in a functional or non-functional coat

In another embodiment, a formulation comprises: i) a core comprising one or more active substances in a modified release, delayed release, controlled release or extended release matrix and ii) substantially surrounded by a coat comprising one or more of the same or different active substance in a functional or non-functional coat.

In accordance with yet another embodiment, a formulation comprises: i) a core comprising one or more active substances in a modified release, delayed release, controlled release or extended release matrix, ii) substantially surrounded by a coat comprising one or more of the same or different active substances in a non-functional coat, and iii) further substantially surrounded by a functional coat.

In accordance with another embodiment, a formulation comprises: i) a core comprising one or more active substances in a modified release, delayed release, controlled release or extended release matrix, ii) substantially surrounded by a coat comprising one or more of the same or different active substances in a functional or non-functional coat, and iii) optionally surrounded by a functional or non-functional coat.

In accordance with another embodiment, a formulation comprises: i) a core comprising one or more active substances in a modified release, delayed release, controlled release or extended release matrix, ii) substantially surrounded by a coat comprising one or more of the same or different active substances in a non-functional coat, and iii) further substantially surrounded by a functional coat that is soluble in gastric fluid up to a pH of about 5 and below but swellable and permeable above a pH of about 5.

In accordance with still another embodiment, a formulation comprises: i) a core comprising one or more active substances with or without a bittering agent in a modified release, delayed release, controlled release or extended release matrix, ii) substantially surrounded by a coat comprising one or more of the same or different active substances in a non-functional coat, and iii) further substantially surrounded by a functional coat that is soluble in gastric fluid up to a pH of about 5 but insoluble above a pH of about 5.0.

In accordance with still another embodiment, a formulation comprises: i) a core comprising one or more active substances with or without a bittering agent, and ii) substantially surrounded by a coat comprising one or more of the same or different active substances in a non-functional coat, and iii) further substantially surrounded by a functional coat that is soluble in gastric fluid up to a pH of about 5 but insoluble above a pH of about 5.0.

In accordance with still another embodiment, a formulation comprises: i) a core comprising one or more active substances and a bittering agent in a modified release, delayed release, controlled release or extended release matrix, ii) substantially surrounded by a coat comprising one or more of the same or different active substances in a non-functional coat, and iii) further substantially surrounded by a functional coat that is soluble in gastric fluid up to a pH of about 5 but swellable and permeable above a pH of about 5.0.

In accordance with still another embodiment, a formulation comprises: i) a core comprising one or more active substances and a bittering agent in a modified release, delayed release, controlled release or extended release matrix, ii) substantially surrounded by a coat comprising one or more of the same or different active substances in a non-functional coat, and iii) further substantially surrounded by a functional coat.

In accordance with still another aspect, the formulation comprises: i) a core comprising one or more active substances and a bittering agent in a modified release, delayed release, controlled release or extended release matrix, and ii) substantially surrounded by a coat comprising one or more of the same or different active substances in a functional or non-functional coat.

In embodiments of the formulations described above, at least one of the functional or non-functional coats applied is from about 1 mg/cm² to about 100 mg/cm²; typically, from about 10 mg/cm² to about 100 mg/cm². The outer most coat is typically 10 mg/cm² to about 100 mg/cnm, or 15 mg/cm² to about 55 mg/cm², or 10 mg/cm² to about 40 mg/cm², or 40 mg/cm² to about 80 mg/cm², or 80 mg/cm² to about 100 mg/cnm². The substance used in the outer most coat typically comprises Eudragit E. However, any suitable polymer may be used that provides pH dependency, ion-exchange dependency, and/or bacterial flora/enzyme dependency and remains substantially intact when about a 350 N force is applied.

In embodiments of the formulations described above, the active substance may be present in any desirable amount. With respect to a maintenance dose, the amount may range from about 1% to about 20% w/w; about 1% to about 10% w/w; or about 2% to about 7% w/w. With respect to a loading dose, the amount may range from about 1% to about 30% w/w; about 1% to about 10% w/w; about 15% to about 30% w/w; or about 1% to about 5% w/w. The substance used in the outer most coat typically comprises Eudragit E.

In accordance with other embodiment, there is provided a formulation whereby one or more active substances are released in a pulsatile manner or in specific sites in the gastrointestinal tract (GIT).

In yet another embodiment, the formulation also has use in other non-medical applications in which the release of a substance is desired into an environment, which eventually comes into contact with fluids. For example, in agriculture, such formulations may be used, for instance, in conjunction with fertilizers, wherein the active ingredient(s) is not released until contacted with specific fluid(s).

In another embodiment, there is provided a formulation with a loading dose that can be effectively employed to control the release of one or more active substances in a formulation or prevent the instantaneous release of most of the dose in the formulations upon perturbation or disruption of the internal and external physical geometries of the formulation.

An immediate release, delayed release, modified release, extended release, pulsed release, sustained release or controlled release profile provided by the formulations disclosed herein may be advantageously used in the formulation of any active ingredient.

A formulation may comprise a core with one or more release retarding agent, controlled release agent, gelling agent, polymeric agents and one or more fillers in a pharmaceutically suitable vehicle, and optionally materials selected from disintegrants, compression aids, lubricants, humectants, surfactants, emulsifiers, plasticizers, anti-oxidants and stabilizers.

A formulation may be formulated such that physicochemical properties reduces or prevents dose dumping of addictive substances in the presence of alcohol, and discourages drug abuse by mode of crushing, milling or grinding the formulation to powder or heating the formulation to vapor and snorting or inhalation by the nasal route or dissolving to abuse via the parenteral route.

A formulation may comprise a core surrounded by a polymeric coat, a plastic coat or elastic coat and the like.

Where a formulation of the present invention comprises more than one coat, a first coat substantially surrounds or envelops a core, a second coat substantially surrounds or envelopes the first coat, and so forth. Coats may take the form and composition of any known compatible controlled-release coat, for example a pH sensitive coat, ion exchange resin coat (Cholestyramine, Colestipol, Sodium polystyrene sulfonate, Polacrilex resin, Polacrilin potassium), intestinal bacteria flora or enzyme reactive polymer (such as a polysaccharide based coat) a water repellant coat, or an aqueous solvent based coat, or a water-soluble coat.

In embodiments, the coating thickness is below 1000 mg/cm², typically below 200 mg/cm² and more typically below 100 mg/cm².

The formulations described herein may release up to about 55% of the total dose as a loading dose to manage pain. In certain embodiments, up to about 55% of the total dose is released as a loading dose within about 60 minutes of ingestion.

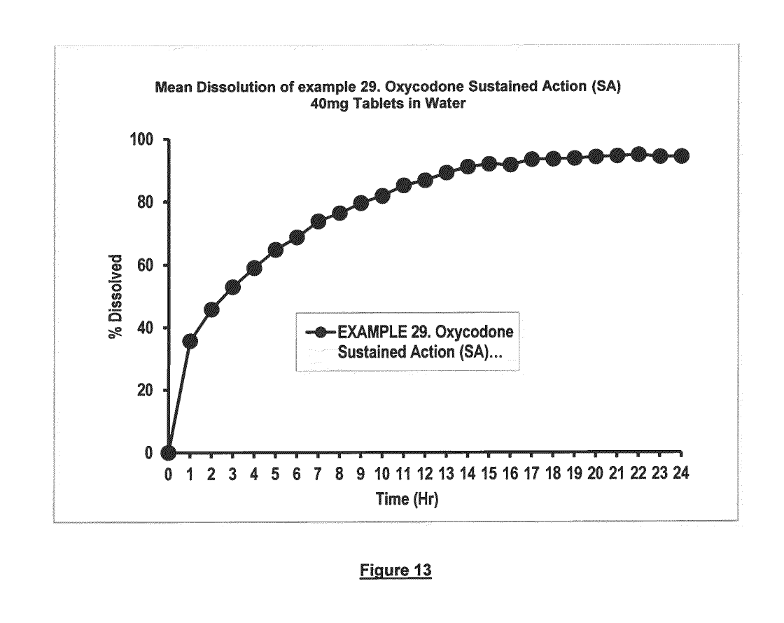

The formulation described herein may also include acid(s) in the coat(s), including an overcoat; layer(s); and/or core of the formulation. Acids such as inorganic and organic acids may be used. Examples include, but are not limited thereto, hydrochloric acid, sulfuric acid, nitric acid, lactic acid, phosphoric acid, citric acid, malic acid, fumaric acid, stearic acid, tartaric acid, boric acid, borax, and benzoic acid. The amount of acid(s) may be present in the formulation in any suitable amount. In some embodiments, the wt % ratio of acid(s) to drug (e.g. in the loading dose and/or maintenance dose) in the formulation is from about 1000:1 to about 1:1000; about 500:1 to about 1:500; or about 1:100 to about 100:1. These ranges, and any ranges mentioned herein, are understood to include any incremental ranges and single amounts encompassed by these ranges. In more specific embodiments, the wt % ratio of acid(s) to loading dose in the formulation is from about 1:100 to about 100:1. In typical embodiments, the acid(s) are organic acids 1:50 to 50:1. In some embodiments, the loading dose may comprise from about 1 wt % to about 1000 wt %; from about 1 wt % to about 500 wt %; from about 1 wt % to about 300 wt %; from about 1 wt % to about 200 wt %; from about 1 wt % to about 100 wt %; from about 1 wt % to about 50 wt %; from about 1 wt % to about 30 wt %; from about 1 wt % to about 20 wt %; or from about 1 wt % to about 15 wt % of the acid(s) based on the weight of the loading dose, whether it be a coat, layer and/or core. In other embodiments, the maintenance dose may comprise from about 1 wt % to about 1000 wt %; from about 1 wt % to about 500 wt %; from about 1 wt % to about 300 wt %; from about 1 wt % to about 200 wt %; from about 1 wt % to about 100 wt %; from about 1 wt % to about 50 wt %; from about 1 wt % to about 20 wt %; or from about 1 wt % to about 15 wt % of the acid(s) based on the weight of the maintenance dose, whether it be a coat, layer and/or core. The amount of acid, typically organic acid(s), may be present in any coat in any suitable amount. In some embodiments, the amount of acid may be from about 5 wt % to less than about 100 wt %, from about 50 wt % to about 90 wt %, from about 50 wt % to about 80 wt %, or from about 60 wt % to about 80 wt % by weight of the coat. In still other embodiments, the coat comprising Eudragit E may comprise from about 1 wt % to about 30 wt %, typically from about wt % to about 25 wt % by weight of the acid(s) based on the weight of the coat. Some organic acid(s) that may particularly be used, for example, and without being limited thereto, lactic acid, phosphoric acid, citric acid, malic acid, fumaric acid, stearic acid, tartaric acid, and benzoic acid. Such acids modify the pH of the macro and micro environment to facilitate release of the active substance. The formulations may have an overcoat comprising at least one acid, typically, at least one organic acid. Typically, such coats comprise at least one acid and a polymer composition such as, but not limited to, Opadry. The amount of acid, typically organic acid(s), may be present in the overcoat in any suitable amount. In certain embodiments, the amount of acid may be from about 5 wt % to less than about 100 wt %, from about 50 wt % to about 90 wt %, typically from about 50 wt % to about 80 wt %, or more typically from about 60 wt % to about 80 wt % by weight of the overcoat. Examples 21 to 40 show specific overcoat examples and FIG. 13 shows the dissolution data for Example 29.

Other formulations may simply comprise at least one primary active substance, at least one coat comprising Eudragit E (dimethylaminoethyl methacrylate copolymer), and at least one coat comprising at least one acid to facilitate release of any active substance in the formulation.

The release profile following crushing of the intact formulation and the controlled release profile of the intact composition may be modified on the basis of many factors pertaining to the formulation, particle size and surface area of the active pharmaceutical ingredient and polymers used, design of the physical geometry of the formulation polymeric coats, for example, without limitation, through the choice of particle size and surface area, types of polymers used, the presence or absence of a loading dose, the order in which they are deposited, the ratios of the loading dose to maintenance dose, the ratios of the polymers in the mix and the nature of their interaction. The controlled-release profile can also be modified by a variety of factors relating to the delivery formulation and the route of administration. For example, the sustained-release period and profile will vary depending upon the loading dose concentration, solubility of the active ingredient, the rate of clearance of the active ingredient from the intended site of administration, the size and surface area of the particle, the amount of the active ingredient initially present in the core, the presence of other compounds within the core that affect the rate of release of the active ingredient, the permeability of the polymeric coating(s) to the active pharmaceutical ingredient, and the rate of degradation of the polymeric coating(s), as well as other factors.

Release control may be effected or optimized through the loading dose, types of polymers used, the number of polymeric coats, the order in which they are deposited, the width of polymeric coats and surface area covered, the ratios of the polymers in the mix and the nature of their interaction.

Incorporating a pharmaceutical drug, that is an addictive substance as described, in the formulation herein may be useful for (1) reducing at least one mode of abuse, for example, the illicit use by snorting/inhalation, parenteral administration, or crushing and oral ingestion of formulations intended for oral administration; (2) reducing dose dumping in the presence of alcohol; or (3) timed or extended release compositions and/or formulations which despite its physical geometry being compromised maintains some or nearly all of its integrity sufficiently to perform controlled release functions during transit in the GIT and (4) potentiation of drug effect due to its ability to deliver quick onset of action followed by sustained action, thus leading to a more effective drug delivery formulation.

The formulations may comprise additives such as Polyethylene Oxide polymers, Polyethylene glycol polymers, Cellulose ethers polymers, Cellulose esters polymers, homo- and copolymers of acrylic acid cross-linked with a polyalkenyl polyether, Poly(meth)acrylates, homopolyers (e.g., polymers of acrylic acid crosslinked with allyl sucrose or allyl pentaerythritol), copolymers (e.g., polymers of acrylic acid and C10-C30 alkyl acrylate crosslinked with allyl pentaerythritol), interpolymers (e.g., a homopolymer or copolymer that contains a block copolymer of polyethylene glycol and a long chain alkyl acid ester), disintegrants, ion exchange resins, polymers reactive to intestinal bacterial flora (e.g., polysaccharides such as guar gum, inulin obtained from plant or chitosan and chondrotin sulphate obtained from animals or alginates from algae or dextran from microbial origin) and pharmaceutical resins.

In some formulations, the core and/or the coat may contain ingredients that, when combined with an aqueous solution, will agglomerate to prevent abuse. Such combinations of ingredients include swellable materials such as PEO and Eudragit RL (or other non-enteric compounds). In general, a formulation may comprise at least one active substance; and at least one excipient, wherein dissolution of the pulverized/milled formulation in alcoholic and/or non-alcoholic beverages causes the formulation to agglomerate.

In some formulations, the core and/or the coat may contain a disintegrant.

Any one of these materials may be present in the formulation or composition in about from 0% to 99% by weight, typically from about 1% to 90% by weight and more typically from 5% to 85%. The typical material is Polyethylene Oxide polymers and acrylic polymers and or their related compounds.

The formulations may optionally comprise a pharmaceutically acceptable nasal irritant such as Capsicum oleoresin. A nasal irritant can produce nasal irritation and annoyance feeling when the composition is brought in contact with the nasal membrane. The irritant agent is not in amounts sufficient to precipitate allergic type reactions or immune response upon snorting. U.S. Pat. No. 7,157,103 suggests the use of various irritants in preparing pharmaceutical formulations including, for example, capsaicin, a capsaicin analog with similar type properties as capsaicin, and the like. Some capsaicin analogues or derivatives include for example, resiniferatoxin, tinyatoxin, heptanoylisobutylamide, heptanoyl guaiacylamide, other isobutylamides or guaiacylamides, dihydrocapsaicin, homovanilyl octylester, nonanoyl vanillylainide, or other compounds of the class known as vaniloids. Resiniferatoxin is described, for example, in U.S. Pat. No. 5,290,816, and U.S. Pat. No. 4,812,446 describes capsaicin analogs and methods for their preparation.

Some examples of controlled release agents that may be used in the formulation of the invention include naturally occurring or synthetic, anionic or nonionic, hydrophobic, hydrophilic rubbers, polymers, starch derivatives, cellulose derivatives, polysaccharides, carbomer, reseins, acrylics, proteins, vinyl-pyrrolidone-vinyl-acetate-copolymers, galactomannan and galactomannan derivatives, carrageenans and the like. Specific examples are acacia, tragacanth, Xanthan gum, locust bean gum, guar-gum, karaya gum, pectin, arginic acid, polyethylene oxide, polyethylene glycol, propylene glycol arginate, hydroxypropyl methylcellulose, methylcellulose, hydroxypropyl cellulose, hydroxyethyl cellulose, carboxymethylcellulose sodium, polyvinylpyrrolidone, carboxyvinyl polymer, sodium polyacrylate, a starch, sodium carboxymethyl starch, albumin, dextrin, dextran sulfate, agar, gelatin, casein, sodium casein, pullulan, polyvinyl alcohol, deacetylated chitosan, polyethyoxazoline, poloxamers, ethylcelulose, chitin, chitosan, cellulose esters, aminoalkyl methacrylate polymer, anionic polymers of methacrylic acid and methacrylates, copolymers of acrylate and methacrylates with quaternary ammonium groups, ethylacrylate methylmethacrylate copolymers with a neutral ester group, polymethacrylates, surfactants, aliphatic polyesters, zein, polyvinyl acetate, polyvinyl chloride, and the like. Further examples of pharmaceutically acceptable acrylic polymers that may also be used include, but are not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolyer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH independent or pH dependent.

Further examples of additives that may be used in the formulation of the invention include, but are not limited to, ethyl lactate, phthalates such as dimethyl phthalate (DMP), diethyl phthalate (DEP), dibutyl phthalate (DBP), dioctyl phthalate, glycol ethers such as ethylene glycol diethyl ether, propylene glycol monomethyl ether, PPG-2 myristyl ether propionate, ethylene glycol monoethyl ether, diethylene glycol monoethyl ether, propylene glycol monotertiary butyl ether, dipropylene glycol monomethyl ether, N-methyl-2-pyrrolldone, 2 pyrrolidone, isopropyl myristate, isopropyl palmitate, octyl palmitate, dimethylacetamide, propylene glycol, propylene glycol monocaprylate, propylene glycol caprylate/caprate, propylene glycol monolaurate, glycofurol, linoleic acid, linoeoyl macrogol-6 glycerides, oleic acid, oleic acid esters such as glyceryl dioleate, ethyl oleate, benzoic acid, oleoyl macrogol-6 glycerides, esters such as ethylbenzoate, benzylbenzoate, sucrose esters, sucrose acetate isobutyrate, esters of lactic acid, esters of oleic acid, sebacates such as dimethyl sebacate, diethyl sebacate, dibutyl sebacate, dipropylene glycol methyl ether acetate (DPM acetate), propylene carbonate, propylene glycol laurate, propylene glycol caprylate/caprate, gamma butyrolactone, medium chain fatty acid triglycerides, glycerol and PEG esters of acids and fatty acids, PEG-6 glycerol mono oleate, PEG-6 glycerol linoleate. PEG-8 glycerol linoleate, caprylic acid esters such as caprylocapryl macrogol-8 glycerides, PEG-4 glyceryl caprylate/caprate, PEG-8 glyceryl caprylate/caprate, polyglyceryl-3-oleate, polyglyceryl-6-dioleate, polyglyceryl-3-sostearate, polyglyceryl polyoleate, decaglyceryl tetraoleate and glyceryl triacetate, glyceryl monooleate, glyceryl monolinoleate, dimethylformamide, dimethylsulfoxide, tetrahydrofuran, caprolactam, decylmethylsutfoxide, and 1-dodecylazacycloheptan-2-one.

The formulation may also contain self-emulsifying or surface active agents with varying hydrophilic lipophilic balance (HLB) values such as polyoxyethylene castor oil, polyoxyethylene hydrogenated castor oil, polyoxyethylene sorbitan fatty acid esters, polyoxyethylene alkyl esters, polyoxyethylene alkyl ethers, polyoxyethylene glycerol esters, sorbitan fatty acid esters, and sodium lauryl sulphate.

Examples of antioxidants that may be used in the formulation is selected from ascorbic acid, fumaric acid, malic acid, a tocopherol, ascorbic acid palmitate, butylated hydroxyanisole, propyl gallate, sodium ascobate, and sodium metabisulfite or other suitable antioxidants and stabilizers.

Examples of plasticizers that may be used in the formulation include adipate, azelate, enzoate, citrate, stearate, isoebucate, sebacate, triethyl citrate, tri-n-butyl citrate, acetyl tri-n-butyl citrate, citric acid esters, and those described in the Encyclopedia of Polymer Science and Technology, Vol. 10 (1969), published by John Wiley & Sons. The typical plasticizers are triacetin, acetylated monoglyceride, acetyltributylcitrate, acetyltriethylcitrate, glycerin sorbitol, diethyloxalate, diethylmalate, diethylphthalate, diethylfumarate, dibutylsuccinate, diethylmalonate, dioctylphthalate, dibutylsebacate, triethylcitrate, tributylcitrate, glyceroltributyrate, polyethylene glycol, glycerol, vegetable and mineral oils and the like. Depending on the particular plasticizer, amounts of from 0 to about 25%, and typically about 0.1% to about 20% of the plasticizer can be used. The addition of plasticizer should be approached with caution. In certain compositions it is better not to use plasticizers.

Examples of other additives that may be used as part of the formulations of the invention include, but are not limited to disintegrants, carbohydrates, sugars, sucrose, sorbitol, mannitol, zinc salts, tannic acid salts; salts of acids and bases such as sodium and potassium phosphates, sodium and potassium hydroxide, sodium and potassium carbonates and bicarbonates; acids such as hydrochloric acid, sulfuric acid, nitric acid, lactic acid, phosphoric acid, citric acid, malic acid, fumaric acid, stearic acid, tartaric acid, boric acid, borax, and benzoic acid.

Organic acid(s) may particularly be used, for example, lactic acid, phosphoric acid, citric acid, malic acid, fumaric acid, stearic acid, tartaric acid, and benzoic acid. Such acids modify the pH of the macro and micro environment to facilitate release of the active substance. The acid(s) may be included in the coat(s), including the overcoat, layer(s), and/or core of the formulation.

Materials such as the alkali metal chlorides, ammonium chloride, and chlorides of Ba, Mg, Ca, Cu, Fe and Al; alkali or alkaline earth solutions of acetates, nitrates, phosphates, and hydroxides may be used in this formulation Hygroscopic or aqueous materials may be used but with caution. Limited quantities may be incorporated in certain compositions.

Water insoluble organosoluble polymers may be used in the formulation, which may be any polymers which are insoluble in water, are capable of being homogenously dissolved or dispersed in an organosolvent, and can typically retard the release of active ingredients. By the term “water-insoluble” is intended not susceptible to being dissolved (in water). Specific examples of water insoluble organosoluble polymers are, cellulose ether, cellulose ester, or cellulose ether-ester e.g., ethyl cellulose, acetyl cellulose, and nitrocellulose. Other water insoluble organosoluble polymers that can be used include acrylic and/or methacrylic ester polymers, polymers or copolymers of acrylate or methacrylate polyvinyl esters, polyvinyl acetates, polyacrylic acid esters, and butadiene styrene copolymers, and the like. Typical water insoluble polymers are, ethylcellulose, cellulose acetate, polymethacrylates and aminoalkyl methacrylate copolymer.

In further specific examples, the acrylic polymer, includes, but is not limited to, acrylic acid and methacrylic acid copolymers, methyl methacrytate copolymers, ethoxyethyl methacrylates, cyanoethyl methacrylate, aminoalkyl methacrylate copolymer, poly(acrylic add), poly(methacrylic acid), methacrylic acid alkylamide copolyer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The water insoluble polymers can be used either singly or in combinations of two or more.

Water-soluble gel forming polymers, which may be used in the formulation, may be any polymers, which are soluble in water, are capable of being homogenously dissolved or dispersed in an organosolvent, and can typically retard the release of active ingredients. Typically, the water-soluble gel-forming polymer is capable of hydrating quickly and forming strong, viscous gels. By the term “water-soluble” is intended susceptible of being dissolved (in water). Suitable water-soluble gel forming polymers include those which can form hydrocolloid or can form a strong, viscous gel through which an active ingredient is released via diffusion or wicking or erosion or swelling. They include naturally occurring or synthetic, anionic or nonlonic, polyethylene oxide, hydrophilic rubbers, starch derivatives, cellulose derivatives, proteins, and the like. Specific non-limiting examples are polyethylene oxide and or its derivatives, gelatin, such as alginates, pectins, carrageenans, or xanthan; cellulose derivatives, such as methyl cellulose, hydroxypropylcellulose, hydroxyethylceflulose, hydroxypropyl methylcellulose, or sodium carboxymethylcellulose; starch and starch derivatives such as a starch or sodium carboxymethyl starch; galactomannan and galactomannan derivatives; polyvinylpyrrolidone, polyvinyl alcohol, polyvinyl acetate and the like, vinyl-pyrrolidone-vinyl-acetate-copolymers, acacia, tragacanth, xanthan gum, locust bean gum, guar-gum, karaya gum, pectin, arginic acid, polyethylene oxide, Carbomer, polyethylene glycols, polypropylene glycols, carboxyvinyl polymer, sodium polyacrylate, albumin, dextrin, dextran sulfate, agar, gelatin, casein, sodium casein, pullulan, deacetylated chitosan, polyethyoxazoline, polyethylene oxide, poloxamers and the like. Of these, typical ones are polyethylene oxide, hydroxyethyl cellulose, hydroxypropyl methylcellulose, methylcellulose, hydroxypropyl cellulose, carbomer, polyethylene glycol, poloxamers, starch derivatives and polyvinylpyrrolidone. Water-soluble gel forming polymers can be used either singly or in combinations of two or more.

Polymeric coats may also be comprised of: hydrophobic or water repellant material such as oils, fats, waxes, higher alcohols; pH sensitive polymers; enteric polymers; or any other polymer, component or material known to be useful for preparing a controlled release coating.

The polymers used in the formulation may be pH insensitive or pH sensitive.

For a delivery formulation designed to be orally administered to the digestive tract, polymers that are known to be orally ingestible can be used and include, for example, polyvinyl alcohol, hydroxypropyl methyl cellulose, and other cellulose-based polymers. Other known polymers useful for enteral delivery include polymer materials, which preferentially dissolve or disintegrate at different points in the digestive tract. Such polymers include, for example, the known acrylic and/or methacrylic acid-based polymers, which are soluble in intestinal fluids, e.g. the Eudragit™ series of commercially available polymers. Examples of these include Eudragit E™, such as Eudragit E 100T, which preferentially dissolves in the more acid pH of the stomach, or enteric polymers such as Eudragit L™ and/or Eudragit S™which preferentially dissolve in the more alkaline pH of the intestine, or polymers which dissolve slowly, e.g. a predetermined rate in the digestive tract, such as Eudragit RL™, e.g. Eudragit RL 100™, and/or Eudragit RS™ e.g. Eudragit R100™, and/or blends of such Eudragit™ polymers.

Polymeric coats may also be comprised of: ion exchange resins and or polymers reactive to intestinal bacterial flora (e.g., polysaccharides such as guar gum, inulin obtained from plant or chitosan and chondrotin sulphate obtained from animals or alginates from algae or dextran from microbial origin)

Hydrophobic or water repellant material that may be present is chosen from oil and fats, waxes, higher fatty acids, fatty acid esters, higher alcohols, hydrocarbons, and metal salts of higher fatty acids. Specific examples of oils and fats include plant oils, e.g. cacao butter, palm oil, Japan wax (wood wax), coconut oil, etc.; animal oils, e.g. beef tallow, lard, horse fat, mutton tallow, etc.; hydrogenated oils of animal origin, e.g. hydrogenated fish oil, hydrogenated whale oil, hydrogenated beef tallow, etc.; hydrogenated oils of plant origin, e.g. hydrogenated rape seed oil, hydrogenated castor oil, hydrogenated coconut oil, hydrogenated soybean oil, etc.; and the like. Of these hydrogenated oils are typical as an oil component of the present invention.

Specific examples of waxes that may be present include plant waxes, e.g. camauba wax, candelilla wax, bayberry wax, auricurry wax, espalt wax, etc.; animal waxes, e.g. bees wax, breached bees wax, insect wax, spermaceti, shellac, lanolin, etc; and the like. Of these typical ones are camauba wax, white beeswax and yellow beeswax.

Paraffin, petrolatum, microcrystalline wax, and the like, are given as specific examples of hydrocarbons, with typical hydrocarbons being paraffin and microcrystalline wax.

Given as examples of higher fatty acids are caprilic acid, undecanoic acid, lauric acid, tridecanic acid, myristic acid, pentadecanoic acid, palmitic acid, malgaric acid, stearic acid, nonadecanic acid, arachic acid, heneicosanic acid, behenic acid, tricosanic acid, lignoceric acid, pentacosanic acid, cerotic acid, heptacosanic acid, montanic acid, nonacosanic acid, melissic acid, hentriacontanic acid, dotriacontanic acid, and the like. Of these, preferable are myristic acid, palmitic acid, stearic acid, and behenic acid.

Specific examples of higher alcohols are lauryl alcohol, tridecyl alcohol, myristyl alcohol, pentadecyl alcohol, cetyl alcohol, heptadecyl alcohol, stearyl alcohol, nonadecyl alcohol, arachyl alcohol, behenyl alcohol, camaubic alcohol, corianyl alcohol, ceryl alcohol, and myricyl alcohol. Particularly preferable alcohols are cetyl alcohol, stearyl alcohol, and the like.

Specific examples of esters are fatty acid esters, e.g. myristyl palmitate, stearyl stearate, myristyl myristate, behenyl behenate, ceryl lignocerate, lacceryl cerotate, lacceryl laccerate, etc.; glycerine fatty acid esters, e.g. lauric monoglyceride, myristic monoglyceride, stearic monoglyceride, behenic monoglyceride, oleic monoglyceride, oleic steeric diglyceride, lauric diglyceride, myristic diglyceride, stearic diglyceride, lauric triglyceride, myristic triglyceride, stearic triglyceride, acetyistearic glyceride, hydoxystearic triglyceride, etc.; and the like. Glycerine fatty acid esters are more typical.

Specific examples of metal salts of higher fatty acid are calcium stearate, magnesium stearate, aluminum stearate, zinc stearate, zinc palmitate, zinc myristate, magnesium myristate, and the like, with preferable higher fatty acid salts being calcium stearate and magnesium stearate.

A polymeric coating composition may also contain other additives such as disintegrants and additives normally found in coatings used in the pharmaceutical art such as plasticizers, anti-tacking agents such as talc and coloring agents.

Coloring agents are added for elegance and aesthetics or to differentiate products and may be chosen, for example, from metal oxide pigments or Aluminum Lake dyes.

A coating composition may include an anti-tacking agent such as talc. Other examples of suitable anti-tacking agent are glycerol monostearate, calcium stearate, colloidal silicon dioxide, glycerin, magnesium stearate, and aluminum stearate.

The compositions are typically formulated to be compatible and result in stable products.

The formulation or composition may be used for treatment of a patient, for example, an animal and more particularly, a mammal. By mammal, is meant any member of the class of Mammalia that is characterized by being a vertebrate having hair and mammary glands. Examples include, without limitation, dog, cat, rabbit, horse, pig, goat, cow, and human being. The formulation or composition of the present invention may be administered to any animal patient or mammalian patient that is in need of treatment with a site specific, timed, pulsed, chronotherapeutic, extended, or controlled release of an active Ingredient. In one example, a delivery formulation of the present invention is used for treating a horse, a dog or a cat. In another example, a delivery formulation of the present invention is used for treating a human being.

A medical condition or dose dumping may be prevented or treated by administering to a patient a formulation or composition comprising a therapeutically effective amount of an addictive substance with quick onset and sustained action of relief.

In certain examples of methods of preparing or using the said formulation or composition, the administration in man or animal may be internal, such as oral or parenteral. Such internal parenteral administration includes but is not limited to intravascular, intramuscular, subcutaneous, intradermal, implantation, and intracavitary routes of administration, as well as application to the external surface of an internal bodily organ, such as during a surgical or laparoscopic procedure. The administration may be topical, including administration to the skin or to a mucosal surface, including the oral, vaginal, rectal surfaces, or to the surface of the eye.

The formulation may also be in the form of a solid. The means and area of application will depend on the particular condition that is being treated. The formulation may be dispensed using any suitable formulation and/or dispensing formulation. For example, it may be taken orally, implanted, or as a depot. It may be targeted at specific sites in the gastrointestinal tract (GU) or to specific organs. As another example, the formulation may also be applied transdermally in a pouch or patch.

Solid particles may be prepared by conventional techniques. They may be milled to required size or surface area where necessary. The typical technique is by dry or wet granulation or hot melt extrusion or roller compaction of an active substance, controlled release agent(s) and excipients such as solubilizing agents, emulsifying agents, suspending agents, fillers, compression agents, stabilizers, pH altering agents, buffers, lubricants, disintegrants and glidants.

Fillers, such as lactose, and compression agents such as microcrystalline cellulose, lubricants such as magnesium stearate and glidants such silicone dioxide may, in certain examples, be included in the core. The core onto which the coating is applied contains the active component. The core may be a tablet, capsule, caplet, pellet, spherical or irregular in shape. The core may be made up of multiple layers by press coating or solution coating. The core may contain a loading dose.

In certain examples, swellable polymeric materials such as hydrogels that swell and expand significantly are included in the core.

Excipients may be homogenously mixed with an active ingredient in a core particle. Excipients may be selected from antiadherents, binders, diluents, emulsifying agents, suspending agents, compression agents, extrusion agents, pH altering agents, buffers, glidants, lubricants, solubilizers, wetting agents, surfactants, penetration enhancers, pigments, colorants, flavoring agents, sweeteners, antioxidants, acidulants, stabilizers, antimicrobial preservatives and binders.

Extrusion agents include, for example, Copolyvidone; copovidone; VPNAc copolymer 60/40; copolymer of 1-vinyl-2-pyrrolidone and vinyl acetate in a ratio of 6:4 by mass, Kollidon VA 64/Fine, Kollidon SR, Kollidon 12/17P, Kollidon 25, Kollidon 30/90, Soluplus (graft copolymer of polyethylene glycol, polyvinyl caprolactam and polyvinylacetate, Cremaphor RH 40.

Excipients are biologically inert ingredients, which enhance the therapeutic effect. The filler or diluent (e.g. lactose or sorbitol) is a bulking agent, providing a quantity of material, which can accurately be formed into a tablet. The binders and adhesives (e.g. methyl cellulose or gelatin) hold the ingredients together so that they form a tablet and hold together. Lubricants (e.g. magnesium stearate or calcium stearate) are added to improve powder flow so that the die fills accurately; they also reduce the friction between the tablet and the machine so that the process progresses smoothly and uniformly.

Anti-adherents are used to reduce the adhesion between the powder (granules) and the punch faces and thus prevent tablet sticking to the punches.

Binders hold the ingredients in a tablet together. Binders ensure that tablets and granules can be formed with required mechanical strength. Binders may be selected from starches, sugars, and cellulose or modified cellulose such as hydroxypropyl cellulose, lactose, or sugar alcohols like xylitol, sorbitol or maltitol. Solution binders are dissolved in a solvent (for example water or alcohol and used in wet granulation processes. Examples of solution binders are gelatin, cellulose, cellulose derivatives, polyvinyl pyrrolidone, starch, sucrose and polyethylene glycol. Dry binders are added to a powder blend, either after a wet granulation step, or as part of a direct powder compression. Examples of dry binders are cellulose, methyl cellulose, polyvinyl pyrrolidone, polyethylene glycol. A commonly used binder or compression agent is microcrystalline cellulose. Microcrystalline and powdered cellulose products are sold under the tradenames Avicel™ PH (FMC Corporation, Philadelphia, Pa.) and Solka Floc™ (Penwest Company, Patterson N.Y.). Microcrystalline cellulose may be used in various techniques such as direct compression, dry granulation, wet granulation, or extrusion-spheronization.

Compression agents are materials that may be compacted. Compression agents may be added to increase the overall hardness of a core particle. Compression agents have inherently high compactibility due to properties of plastic deformation and limited elastic recovery. Non-limiting examples of materials that find use as compression agents are microcrystalline cellulose, silicified microcrystalline cellulose (for example Prosolv™ produced by JRS Pharma), oxidized polyethylene, calcium hydrogen phosphate dehydrate, dextrate, or sugar.

Fillers or diluents are added for bulk to fill out the size of a tablet or capsule, making it practical to produce and convenient for the consumer to use. Fillers/diluents are typically inert, compatible with the other components of the formulation, non-hygroscopic, soluble, relatively cheap, compactible, and typically tasteless or pleasant tasting. Plant cellulose (pure plant filler) is a popular filler in tablets or hard gelatin capsules. Dibasic calcium phosphate is another popular tablet filler. A range of vegetable fats and oils can be used in soft gelatin capsules.

Other examples of fillers include: lactose, sucrose, glucose, mannitol, sorbitol, and, calcium carbonate. Fillers/dluents are typically selected from microcrystalline cellulose, plant cellulose, calcium phosphate, mannitol, sorbitol, xylitol, glucitol, ducitol, inositiol, arabinitol; arabitol, galactitol, iditol, allitol, fructose, sorbose, glucose, xylose, trehalose, allose, dextrose, altrose, gulose, idose, galactose, talose, ribose, arabinose, xylose, lyxose, sucrose, maltose, lactose, lactulose, fucose, rhamnose, melezitose, maltotriose, and raffinose. Typical sugars include mannitol, lactose, sucrose, sorbitol, trehalose, glucose.

Glidants are used to improve the flowability of the powder or granules or both. Some examples of glidant(s) are silicon dioxide, starch, calcium silicate, Cabosil, Syloid, and silicon dioxide aerogels. Typically, silicon dioxide is used.

Lubricants prevent ingredients from clumping together and from sticking to the tablet punches or capsule-filling machine. Lubricants also ensure that tablet formation and injection can occur with low friction between the solid and die wall. Some examples of lubricant(s) are alkali stearates such as magnesium stearate, calcium stearate, zinc stearate, polyethylene glycol, adipic acid, hydrogenated vegetable oils, sodium chloride, sterotex, glycerol monostearate, talc, polyethylene glycol, sodium benzoate, sodium lauryl sulfate, magnesium lauryl sulfate, sodium stearyl fumarate, light mineral oil and the like may be employed. Waxy fatty acid esters, such as glyceryl behenate, sold as “Compritol” products, can be used. Other useful commercial lubricants include “Stear-O-Wet” and “Myvatex TL”. Common minerals like talc or silica, and fats, e.g. vegetable stearin, glycerol monostearate, magnesium stearate or stearic acid are typically used lubricants.

Sorbents are used for moisture proofing by limited fluid sorbing (taking up of a liquid or a gas either by adsorption or by absorption) in a dry state.

Surfactants, wetting agents and solubilisers such as glycerol monostearate, cetostearyl alcohol, cetomacrogol emulsifying wax, sorbitan esters, polyoxyethylene alkyl ethers (e.g., macrogol ethers such as cetomacrogol 1000), polyoxyethlylene castor oil derivatives, polyoxyethylene sorbitan fatty acid esters (e.g., TWEEN™), polyoxyethylene stearates, sodium dodecylsulfate, Tyloxapol (a nonionic liquid polymer of the alkyl aryl polyether alcohol type, also known as superinone or triton) is another useful solubilisers. Most of these solubilisers, wetting agents and surfactants are known pharmaceutical excipients and are described in detail in the Handbook of Pharmaceutical Excipients, published jointly by the American Pharmaceutical Association and The Pharmaceutical Society of Great Britain (The Pharmaceutical Press, 1986).

Typical wetting agents include tyloxapol, poloxamers such as PLURONIC™ F68, F127, and F108, which are block copolymers of ethylene oxide and propylene oxide, and polyxamines such as TETRONIC™ 908 (also known as POLOXAMINE™ 908), which is a tetrafunctional block copolymer derived from sequential addition of propylene oxide and ethylene oxide to ethylenediamine (available from BASF), dextran, lecithin, dialkylesters of sodium sulfosuccinic acid such as AEROSOL™ OT, which is a dioctyl ester of sodium sulfosuccinic acid (available from American Cyanimid), DUPONOL™ P, which is a sodium lauryl sulfate (available from DuPont), TRITON™ X-200, which is an alkyl aryl polyether sulfonate (available from Rohm and Haas), TWEEN™ 20 and TWEEN^{M} 80, which are polyoxyethylene sorbitan fatty acid esters (available from ICI Specialty Chemicals), Carbowax 3550 and 934, which are polyethylene glycols (available from Union Carbide), Crodesta F-110, which is a mixture of sucrose stearate and sucrose distearate, and Crodesta SL-40 (both available from Croda Inc.), and SA90HCO, which is Cg₁₈H₃₇—CH₂ (CON(CH₃)CH₂(CHOH) CF₂₀H)₂.

Wetting agents which have been found to be particularly useful, include Tetronic 908, the Tweens, Pluronic F-68 and polyvinylpyrrolidone. Other useful wetting agents include decanoyl-N-methylglucamide; n-decyl-β-D-glucopyranoside; n-decyl-β-D-maltopyranoside; n-dodecyl-β-D-glucopyranoside; n-dodecyl-β-D-maltoside; heptanoyl-N-methylglucamide; n-heptyl-β-D-glucopyranoside; n-heptyl-β-D-thioglucoside; n-hexyl-β-D-glucopyranoside; nonanoyl-N-methylglucamide; n-octyl-β-D-glucopyranoside; octanoyl-N-methylglucamide; n-octyl-β-D-glucopyranoside; and octyl-β-D-thioglucopyranoside. Another typical wetting agent is p-isononylphenoxypoly(glycidol), also known as Olin-10G or Surfactant 10-G (commercially available as 10G from Olin Chemicals). Two or more wetting agents can be used in combination.

The pharmaceutical formulation or formulation may further include a pegylated excipient. Such pegylated excipients include, but are not limited to, pegylated phospholipids, pegylated proteins, pegylated peptides, pegylated sugars, pegylated polysaccharides, pegylated block-co-polymers with one of the blocks being PEG, and pegylated hydrophobic compounds such as pegylated cholesterol. Representative examples of pegylated phospholipids include 1,2-diacyl 1-sn-gycero-3-phosphoethanolamine-N-[Poly(ethylene glycol) 2000](“PEG 2000 PE”) and 1,2-diacyl-sn-glycero-3-phosphoethanolamine-N-[Poly(ethylene glycol) 5000](“PEG 5000 PE”), where the acyl group is selected, for example, from dimyristoyl, dipalmitoyl, distearoyl, diolcoyl, and 1-palmitoyl-2-oleoyl.

Additional excipients may be included in the formulation of the present invention. Further examples of excipients can include pigments, colorants, flavoring agents, preservatives and sweetners. Flavors and colors are added to improve the taste or appearance of a formulation. Some typical preservatives used in pharmaceutical formulations are antioxidants such as vitamin A, vitamin E, vitamin C, and selenium, amino acids such as cysteine and methionine, citric acid and sodium citrate, or synthetic preservatives such as methyl paraben and propyl paraben. Sweeteners are added to make the ingredients more palatable, especially in chewable tablets such as antacid or liquids like cough syrup. Sugar may be used to disguise unpleasant tastes or smells. While for addictive substances bittering agents may be added make the administration of a non-intact form objectionable.

One skilled in the art can select appropriate exciplents for use in the formulation of the present invention.

The formulation may comprise an excipient that is a swellable material such as a hydrogel in amounts that can swell and expand. Examples of swellable materials include polyethylene oxide, hydrophilic polymers that are lightly cross-linked, such cross-links being formed by covalent or ionic bond, which interact with water and aqueous biological fluids and swell or expand to some equilibrium state. Swellable materials such as hydrogels exhibit the ability to swell in water and retain a significant fraction of water within its structure, and when cross-linked they will not dissolve in the water. Swellable polymers can swell or expand to a very high degree, exhibiting a 2 to 50 fold volume increase. Specific examples of hydrophilic polymeric materials include poly(hydroxyalkyl methacrylate), poly(N-vinyl-2-pyrrolidone), anionic and cationic hydrogels, polyelectrolyte complexes, poly(vinyl alcohol) having a low acetate residual and cross-linked with glyoxal, formaldehyde, or glutaraldehyde, methyl cellulose cross-linked with dialdehyde, a mixture of cross-linked agar and carboxymethyl cellulose, a water insoluble, water-swellable copolymer produced by forming a dispersion of finely divided copolymer of maleic anhydride with styrene, ethylene, propylene, butylene, or isobutylene cross-linked with from 0.001 to about 0.5 moles of a polyunsaturated cross-linking agent per mole of maleic anhydride in the copolymer, water-swellable polymers of N-vinyl lactams, cross-linked polyethylene oxides, and the like. Other examples of swellable materials include hydrogels exhibiting a cross-linking of 0.05 to 60%, hydrophilic hydrogels known as Carbopol acidic carboxy polymer, Cyanamer™ polyacrylamides, cross-linked water-swellable indene-maleic anhydride polymers, Good-rite™ polyacrylic acid, starch graft copolymers, Aqua-Keeps™ acrylate polymer, diester cross-linked polyglucan, and the like. Methods for testing swellable materials with regards to polymer imbibition pressure and hydrogel-water interface interaction are described in U.S. Pat. No. 4,327,725.

In a certain example, the formulation may be coated with salt forming, and/or ion exchanging resin, and/or a non-disintegrating and/or non-semi-permeable coat. Materials useful for forming the non-disintegrating non-semi-permeable coat are ethylcellulose, polymethylmethacrylates, methacrylic acid copolymers and mixtures thereof.

In yet another embodiment, the formulation is coated with a non-disintegrating semipermeable coat. Materials useful for forming the non-disintegrating semipermeable coat are cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. Other suitable polymers are described in U.S. Pat. Nos. 3,845,770, 3,916,899, 4,008,719, 4,036,228 and 4,612,008. The most typical non-disintegrating semipermeable coating material is cellulose acetate comprising an acetyl content of 39.3 to 40.3%, commercially available from Eastman Fine Chemicals.

In an alternative embodiment, the non-disintegrating semipermeable or non-disintegrating non-semi-permeable coat can be formed from the above-described polymers and materials that will form pores or channels in the coat. The pore forming agents or channeling agents dissolve on contact with fluid and form passages through which fluid and active pharmaceutical ingredient(s) can move through the coat. The pore forming agent or channeling agent can be a water-soluble material or an enteric material. Some general examples of pore forming agents or channeling agents are water soluble materials such as cellulose ethers, polyethylene glycols or microcrystalline cellulose. Some further examples of pore forming agents or channeling agents are sodium chloride, potassium chloride, lactose, sucrose, sorbitol, mannitol, polyethylene glycol (PEG), for example PEG 600, polyvinyl pyrolidone, propylene glycol, hydroxypropyl cellulose, hydroxypropyl methycellulose, hydroxypropyl methycellulose phthalate, cellulose acetate phthalate, polyvinyl alcohols, methacrylic acid copolymers and mixtures thereof.

The active pharmaceutical ingredient(s) that are water-soluble or that are soluble under intestinal conditions may also be used to create pores in the coat.

The pore forming agent comprises approximately 0 to about 75% of the total weight of the coating, most typically about 0.5% to about 25% of the total weight of the coating. The pore-forming agent dissolves or leaches from the coat to form pores in the coat for the fluid to enter the core and dissolve the active ingredient.

As used herein the term pore includes an aperture, orifice, bore, channel, hole, a discrete area of weakness or as created by soluble or leachable materials.

Certain general illustrative examples of the formulation or formulations and their uses may be helpful in understanding the present invention and are itemized as follows:

Item 1 is the formulation, which provides zero order release; first order or pseudo-first order release of active substance content after a loading dose has been released.

Item 2 is the formulation which releases less than 60% of active substance in 1 hour using USP basket dissolution apparatus at 100 rpm.

Item 3 is the formulation which releases between 30% to 40% of active substance in 1 hour using USP basket dissolution apparatus 100 rpm.

Item 4 is the formulation which releases between 20% to 30% of active substance in 1 hour using USP basket dissolution apparatus at 100 rpm.

Item 5 is the formulation which provides pulsed delivery.

Item 6 is the formulation which provides chronotherapeutic delivery.

Item 7 is the formulation comprising an active substance and one or more materials selected from the group polyethylene oxide, disintegrant, acrylic polymer, for preventing dose dumping in the presence of alcohol and which makes it difficult for drug abuse.

Item 8 is the formulation comprising active substances with small particle size or large surface area and one or more materials selected from the group polyethylene oxide, disintegrant, acrylic polymer, for preventing dose dumping in the presence of alcohol and or which makes it difficult for drug abuse.

Item 9 is the formulation according to items 1 to 8 which is presented as tablet, pellet, bead, microsphere, nanoparticle or granules

Item 10 is the formulation according to items 1 to 9 for pediatric, adult or geriatric use. Item 11 is the formulation according to items 1 to 10 for use as an implant or subcutaneous application.

Item 12 is the formulation according to items 1 to 11 wherein dissolution using a USP dissolution tester is not significantly affected by the rotation speed of the basket or paddle in the speed range from about 25 rpm to about 150 rpm at least in the first hour.

Item 13 is the formulation according to items 1 to 11 wherein dissolution using a USP dissolution tester is not significantly affected by the rotation speed of the basket or paddle in the speed range from about 50 rpm to about 150 rpm at least in the first hour.

Item 14 is the formulation according to items 1 to 11 wherein dissolution using a USP dissolution tester is not significantly affected by the rotation speed of the basket or paddle in the speed range from about 50 rpm to about 100 rpm at least in the first hour.

Item 15 is the formulation according to items 1 to 11 wherein dissolution using a USP dissolution tester is not significantly affected by the rotation speed of the basket or paddle in the speed range from about 50 rpm to about 75 rpm at least in the first hour.

Item 16 is the formulation according to items 1 to 11 wherein there is less or no dose dumping during dissolution using a USP dissolution tester with basket or paddle assembly in alcoholic media.

Item 17 is the formulation according to items 1 to 11 wherein there is less or no dose dumping during dissolution using a USP dissolution tester with basket or paddle assembly at 50 or 100 rpm in about 1% to about 10% alcoholic media.

Item 18 is the formulation according to items 1 to 11 wherein there is no dose dumping during dissolution using a USP dissolution tester with basket or paddle assembly at 50 or 100 rpm in about 10% to about 20% alcoholic media.

Item 19 is the formulation according to items 1 to 11 wherein there is no dose dumping during dissolution using a USP dissolution tester with basket or paddle assembly at 50 or 100 rpm in about 20% to about 30% alcoholic media.

Item 20 is the formulation according to items 1 to 11 wherein there is no dose dumping during dissolution using a USP dissolution tester with basket or paddle assembly at 50 or 100 rpm in about 30% to about 40% alcoholic media.

Item 21 is the formulation according to items 1 to 11 wherein there is no dose dumping during dissolution using a USP dissolution tester with basket or paddle assembly at 50 or 100 rpm in about about 40% to about 50% alcoholic media.

Item 22 is the formulation according to items 1 to 11 wherein there is no dose dumping during dissolution using a USP dissolution tester with basket or paddle assembly at 50 or 100 rpm in about 50% to about 70% alcoholic media.

Item 23 is the formulation containing one or mixture of medicaments used to manage pain, medicaments used to reduce or eliminate anxiety attack (psychotherapeutic drugs), medicaments that are used as stimulants and sleeping pills, cardiovascular agents, antidiabetics, acid labile drugs and in general medicaments (whose intended effects may be compromised if the intact formulation is heated, burned, microwaved, frozen, perturbed, pulverized or crushed or ground or milled or cut into one or a mixture of sizes ranging from very fine to coarse particles, granules or spheres) which provides zero order release; first order or pseudo-first order release of drug content after a loading dose has been released.

Item 24 is the formulation according to items 1 to 11 and 23 wherein it is enveloped in a coat. The coat may be a pod-like structure or cocoon.

Item 25 is the formulation according to item 24 wherein the pod-like structure or cocoon is made from acrylic polymer and/or polysaccharide and/or ion exchange resin.

Item 26 is the formulation according to item 24 wherein the pod-like structure or cocoon is made from an acrylic polymer and/or polysaccharide and/or ion exchange resin and other pharmaceutically acceptable polymer.

Item 27 is the formulation according to item 24 wherein the pod like structure or cocoon is made from acrylic polymer which dissolves by salt formation at acid pH.

Item 28 is the formulation according to item 27 wherein the loading dose is released after the coat (e.g. pod like structure or cocoon) dissolves.

Item 29 is the formulation according to item 24 wherein multiple peak plasma concentrations are observed on oral administration.

### Method of Making the Formulations

The formulations can be made by any known methods. For example, the core can be made by blending and direct compression without wet granulation; by hot melt extrusion; by hot melt granulation; by roll compaction, slugging or a chilsonator; and/or by extrusion spheronization. The loading dose or any coating may be press coated onto at least a portion of the core as a separate layer(s).

In some embodiments, the loading dose is applied by spraying coating, dry coating, press coating, encapsulation, or by a combination of these methods.

In a specific example, a polymeric coating is prepared by adding polymers, plasticizer, and anti-tacking agent to an organosolvent or aqueous system and mixed until homogenously dissolved or dispersed using a low or high shear mixer. The coating may be applied to a core using standard coating methodology.

In a certain embodiment, there is provided a method for making a loading dose for a formulation, which comprises hot melt extruding a loading dose, wherein the loading dose comprises at least one active substance and at least one excipient; and incorporating the loading dose in the formulation. Hot melt extrusion is advantageous when utilizing insoluble material/components.

The formulations described herein may contain one or more active substance, or specifically one or more opioid agonist or narcotic analgesic or abuse-able substances, may be made by any method wherein the particle size or surface area of active ingredient and/or inactive ingredient, quantity or ratio and type of loading dose, controlled release agents, external coat(s) and excipients is optimum to form a formulation with quick onset of action and sustained action thereafter while still capable of abuse resistant properties when crushed.

Typically, the entire quantity of the core formulation is dry mixed and homogeneously blended, and made into a solid unit (e.g. tablet, bead, compressed granules formed into any shape, etc.). Thereafter, the loading dose or a portion of it is applied directly on the core by press coating as a layer, for example, on top of one side of the unit or solution coating, surrounding or almost completely surrounding the tablet A cold process under room temperature conditions is typical, however solid substances may be heated to their liquid state prior to incorporation, using such methods as hot melt extrusion.

Alternatively, the formulation may be processed in a jacketed vessel, which allows precise control of the processing temperature. Other pharmaceutically acceptable additives, such as those described above, may be incorporated before, after, or during the addition of controlled release agents or narcotic analgesics. Wet granulation can also be used.

The solid particles may be of a size and/surface area such that the active ingredient maintains very intimate and close proximity to the polymers and homogeneity. The solid particles may take any convenient form, including, for example, granules, spheroids, pellets, microspheres, nanospheres, microcapsules, or crystals and can be prepared by wet or dry granulation, by extrusion spheronization, by hot melt extrusion, by powder or solution layering, by microencapsulation techniques, by milling and compression techniques or other suitable known techniques. In certain examples, different types of coats may be applied to the formulation.

In certain examples, the particle size of solid materials is less than about 1000 microns. In certain other examples, the particle size of solid materials is less than about 500, 200. 100, or 50 microns and the formulation maintains very Intimate and close proximity to the polymers and homogeneity especially when crushed. In certain further, examples the solid particles are sufficiently small and have large surface area such that they are in very intimate and close proximity and homogeneity with one another. These types of formulations may resist abuse or inadvertent misuse.

In certain examples, capsules, for example, soft or hard capsules, envelop the formulations. While both soft and hard capsules may be used, hard capsules may be particularly useful. In certain examples, the capsule is made by applying a polymeric coat of material that result in a plastic or elastic shell in any shape (e.g. pod-like envelope). It could also be a hard gelatin capsule or is made of a metal or alloy of metals, cellulose ether, vegetable or animal origin.

One skilled in the art will also know that capsules made from materials other than gelatin may be used. For example, U.S. Patent Application Publication No. 2006/0099246 pertains to a non-gelatin soft capsule system having a predominantly starch and gelling carrageenan based shell. Carrageenan is a collective term for polysaccharides prepared by alkaline extraction (and modification) from red seaweed (Rhodophycae), mostly of genus Chondrus, Eucheuma, Gigartina and Iridaea. Different seaweeds produce different carrageenans. Carrageenan consists of alternating 3-linked-β-D-galactopyranose and 4-linked-α-D-galactopyranose units. Most, if not all, of the galactose units are substituted with sulfate ester groups. In another example, US Patent Appln. Pub. No. 2006/0004193 (Muller) published Jan. 5, 2006 relates to a tough-elastic material based on starch, which on the one hand has high impact toughness at low humidity, and on the other hand still has a high modulus of elasticity at high humidity and has a high elongation capacity in a broad range of humidity and on account of its property profile is suited to use as edible film and for the packaging of active ingredients, as well as high-quality substitution of gelatin in the area of soft and hard capsules. As another example, PCT Publication WO 01/37817 describes a soft capsule based on thermoplastic starch (TPS) with high softener content. As another example, U.S. Patent Application Publication No. 2005/0196436 relates to a method of producing a film-forming blend of different acyl gellan gums with starch having similar textural and functional properties compared to gelatin. As another example, U.S. Patent Application Publication No. 2007/0077293 (Park) published Apr. 5, 2007 relates to a film-forming composition for hard capsules, comprising 7-12% by weight of starch, 1-6% by weight of a plasticizer, 0.7-3% by weight of a gelling agent, and 79-91.3% by weight of water. As another example, U.S. Patent Application Publication No. 2006/0153909 relates to hard capsules made of a base material containing a cellulose derivative including, for example, one or more of hydroxypropyl methylcellulose, methylcellulose, hydroxypropyl cellulose, hydroxyethyl cellulose, hydroxypropyl methylcellulose phthalate, hydroxypropyl methylcellulose acetate succinate, carmelose, carboxymethylethyl cellulose, cellulose acetate phthalate, and ethylcellulose. Also, additives such as a gelling agent, a gelling aid, a colorant, a plasticizer, an emulsifier, a dispersant, and a preservative may be added to the capsule base material. As yet another example, U.S. Patent Application Publication No. 2005/0186268 describes a hard capsule made mainly of a polymer or copolymer obtained by polymerizing or copolymerizing at least one polymerizable vinyl monomer in the presence of polyvinyl alcohol and/or a derivative thereof. Still many other examples exist, as will be recognized by the skilled person.

In certain examples, a controlled release formulation may be in combination with a non-controlled release formulation containing opioid antagonist and/or immediate release non-narcotic analgesics or other pharmaceutically active substances or filled into a capsule or dispensing formulation with non-controlled release composition containing opioid antagonist and/or immediate release non-narcotic analgesics or other pharmaceutically active substances.

In certain examples, dissolution using a USP dissolution tester is not significantly different by the rotation speed of the basket or paddle in the speed range from about 25 rpm to about 150 rpm, or at about 50 rpm and about 100 rpm or at about 50 rpm and about 75 rpm or at about 100 rpm and about 150 rpm. The rotation speed does not interact with or compromise the integrity of the formulation and release mechanism, particularly in the first hour. Formulations that meet these requirements perform consistently in the gastrointestinal tract without fear of collapse or disintegration. These are typically not perturbed, crushed or damaged by gastrointestinal tract content, resident time or motility.

When introducing elements disclosed herein, the articles “a”, “an”, “the”, and “said” are intended to mean that there may be one or more of the elements.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient Although specific terms have been employed herein, such terms are intended in a descriptive sense and not for purposes of limitation.

### EXAMPLES

### Example 1

### Propranolol Sustained Action (SA) 80 mg Tablets

### (80 mg Tablets Contain 70 mg Maintenance Dose and 10 mg Loading Dose)

### Formula for Maintenance Dose

| Ingredients | % w/w |
|---|---|
| Propranolol | 20.00 |
| (particle size 400 microns) | |
| Polyethylene Oxide | 45.00 |
| (particle size <600 | |
| microns) | |
| Lactose | 10.00 |
| Pregelatinized starch | 5.00 |
| Microcrystalline cellulose | 14.00 |
| Eudragit RL | 5.00 |
| Magnesium stearate | 1.00 |

### Formula for Loading Dose

| Ingredients | % w/w |
|---|---|
| Propranolol | 5.00 |
| (particle size 400 microns) | |
| Lactose | 60.00 |
| Hydroxypropyl methyl | 4.00 |
| cellulose | |
| Pregelatinized starch | 5.00 |
| Microcrystalline cellulose | 20.00 |
| Magnesium stearate | 1.00 |

### Formula for the Pod-like Envelope

| Ingredients | % w/w |
|---|---|
| Eudragit E | 52.63 |
| Talc | 22.10 |
| Magnesium stearate | 3.23 |
| Titanium dioxide | 18.94 |
| Polyethylene glycol 6000 | 3.10 |
| Water | qs |
| Isopropyl alcohol/ | qs |
| Acetone | |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with exception of the magnesium stearate and pregelatinized starch from the loading dose formula were charged into a high shear granulator and dry mixed for less than 10 minutes. The dry mixed granules were discharged into a Paterson Kelly V-Blender. The magnesium stearate and pregelatinized starch were then added to the V-Blender. The granules were blended for less than 10 minutes.

Step 2. Preparation of a bi-layer tablet containing maintenance dose and loading dose: The first layer is made from the granules prepared in Step 1a, and the second layer is made from granules prepared in Step 1 b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3 Preparation of a Coating Suspension of the Ingredients of the Pod-Like Envelope Applied to the Bi-Layer Tablet:

(I) Isopropyl alcohol was added into a stainless steel vessel followed by Eudragit E, titanium dioxide, talc and magnesium stearate, step-by-step, while stirring vigorously with a high shear mixer until all ingredients were finely dispersed in a suspension. (II) Polyethylene glycol was dissolved in water. (III) The polyethylene glycol water mixture was added to the Eudragit E suspension while stirring using a high shear mixer.
Step 4. Application of the Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 20 mg/cm² to about 30 mg/cm² of the coat surrounding the bi-layer tablet.

### Example 2

### Hydromorphone Sustained Action (SA) 8 mg Tablets

### (8 mg Tablets Contain 7 mg Maintenance Dose and 1 mg Loading Dose)

### Formula for Maintenance Dose

| Ingredients | % w/w |
|---|---|
| Hydromorphone | 2.00 |
| (particle size 600 microns) | |
| Polyethylene Oxide | 40.00 |
| (particle size <600 | |
| microns) | |
| Lactose | 28.00 |
| Pregelatinized starch | 5.00 |
| Microcrystalline cellulose | 14.00 |
| Eudragit RL | 5.00 |
| Carbomer | 5.00 |
| Magnesium stearate | 1.00 |

### Formula for Loading Dose

| Ingredients | % w/w |
|---|---|
| Hydromorphone | 1.00 |
| (particle size 600 microns) | |
| Lactose | 65.00 |
| Hydroxypropyl methyl | 4.00 |
| cellulose | |
| Pregelatinized starch | 5.00 |
| Microcrystalline cellulose | 20.00 |
| Magnesium stearate | 1.00 |

### Formula for the Pod-Like Envelope

| Ingredients | % w/w |
|---|---|
| Eudragit E | 74.50 |
| Citric Acid | 25.50 |
| Water | qs |

### Processing Techniques

Step 1a. Preparation of Granules for the Maintenance Dose:

All the ingredients with exception of the magnesium stearate from the maintenance dose formula were charged into a high shear granulator and dry mixed for less than 10 minutes. The dry mixed materials were discharged into a Roll Compactor and the materials were forced between two counter rotating rolls in the Roll Compactor in order to form flakes or compacts. The compacts were granulated to reduce their size to uniform particle size distribution by passing them through a size reduction mill fitted with rotating blades and a perforated screen. The granules were discharged into a Paterson Kelly V-Blender. The magnesium stearate was added to the granules in the V-Blender and blend for less than 10 minutes.

Step 1b. Preparation of Granules for the Loading Dose:

All the ingredients, with the exception of the magnesium stearate and pregelatinized starch, from the loading dose formula were discharged into a high shear granulator and dry mixed for less than 10 minutes. The dry mixed materials were discharged into a Roll Compactor and the materials were forced between the two counter rotating rolls in the Roll Compactor in order to form flakes or compacts. The compacts were granulated to reduce their size to uniform particle size distribution by passing them through a size reduction mill fitted with rotating blades and perforated screen. The granules were discharged into a Paterson Kelly V-Blender. The magnesium stearate and pregelatinized starch were added to the granules in the V-Blender and blended for less than 10 minutes.

Step 2. Preparation of a bi-layer tablet containing maintenance dose and loading dose: The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of a Coating Suspension of the Ingredients of the Pod-Like Envelope Applied to the Bi-Layer Tablet:

(I) Water was added into a stainless steel vessel and Eudragit E was gradually added while stirring with a high shear mixer with controlled speed, sufficient to prevent sedimentation and lump formation. (II) The citric acid portion was added until a clear solution is obtained.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 10 mg/cm² to about 20 mg/cm² of the coat surrounding the bi-layer tablet.

### Example 3

### Morphine Sustained Action (SA) 30 mg Tablets

### (30 mg Tablets Contain 25 mg Maintenance Dose and 5 mg Loading Dose)

### Formula for Maintenance Dose

| Ingredients | % w/w |
|---|---|
| Morphine (particle | 5.00 |
| size 1000 microns) | |
| Polyethylene Oxide | 70.00 |
| (particle size 1000 | |
| microns) | |
| Crospovidone | 2.00 |
| Microcrystalline cellulose | 5.00 |
| Eudragit RL | 5.00 |
| Capsicum oleoresin | 5.00 |
| Magnesium stearate | 1.00 |

### Formula for Loading Dose

| Ingredients | % w/w |
|---|---|
| Opadry | 83.00 |
| Capsicum oleoresin | 3.35 |
| Morphine | 16.65 |
| Water | qs |

### Formula for the Pod-Like Envelope

| Ingredients | % w/w |
|---|---|
| Eudragit E (milled) | 59.29 |
| Sodium Lauryl sulfate | 5.93 |
| Stearic acid (milled) | 8.89 |
| Talc | 25.89 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied to the Tablet:

(I) Water was added into a stainless steel vessel. (II) Opadry was added while stirring with a propeller mixer until all ingredients are finely dispersed in a suspension. (III) Morphine and Capsicum oleoresin was added to the Opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

Step 5. Preparation of a Coating Suspension of the Ingredients for a Pod-Like Envelope was Applied to the Coated Tablet from Step 4:

(I) Water was added to a stainless steel vessel, followed by Sodium lauryl sulfate and stearic acid, step-by-step, while stirring vigorously with a high shear mixer until all ingredients are dissolved. (II) Eudragit E was added step-by-step while stirring vigorously with a high shear mixer until all ingredients were dissolved. (III) Talc was added while stirring using a high shear mixer until finely dispersed in the solution.

Step 6. Application of the Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 40 mg/cm² to about 50 mg/cm² of the coat surrounding the coated tablet.

### Example 4

### Codeine Sustained Action (SA) 30 mg Tablets

### (30 mg Tablets Contain 26 mg Maintenance Dose and 5 mg Loading Dose)

### Formula for Maintenance Dose

| Ingredients | % w/w |
|---|---|
| Codeine | 5.00 |
| (particle size 600 microns) | |
| Polyethylene Oxide | 40.00 |
| (particle size <600 | |
| microns) | |
| Lactose | 25.00 |
| Pre-gelatinized starch | 5.00 |
| Microcrystalline cellulose | 19.00 |
| Eudragit RL | 5.00 |
| Magnesium stearate | 1.00 |

### Formula for Loading Dose

| Ingredients | % w/w |
|---|---|
| Codeine | 2.50 |
| (particle size 600 microns) | |
| Lactose | 37.00 |
| Hydroxypropyl | 2.00 |
| methylcellulose | |
| Crospovidone | 7.50 |
| Microcrystalline cellulose | 24.50 |
| Stearic acid | 1.50 |

### Formula for the Pod-Like Envelope

| Ingredients | % w/w |
|---|---|
| Eudragit E | 52.63 |
| Talc | 22.10 |
| Magnesium stearate | 3.23 |
| Titanium dioxide | 18.94 |
| Polyethylene glycol 6000 | 3.10 |
| Water | qs |
| Isopropyl alcohol | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Isopropyl alcohol was added into a stainless steel vessel followed by Eudragit E, titanium dioxide, talc and magnesium stearate, step-by-step, while stirring vigorously with a high shear mixer until all ingredients were finely dispersed in a suspension. (II) Polyethylene glycol was dissolved in water. (III) The polyethylene glycol water mixture was added to the Eudragit E suspension while stirring using a high shear mixer.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 20 mg/cm² to about 30 mg/cm² of the coat surrounding the bi-layer tablet.

### Example 6

### Oxymorphone Sustained Action (SA) Tablets

### (30 mg Tablets Contain 25 mg Maintenance Dose and 5 mg Loading Dose)

### Formula for Maintenance Dose

| Ingredients | % w/w |
|---|---|
| Oxymorphone | 5.00 |
| Polyethylene Oxide | 76.00 |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 83.00 |
| Sucrose Octaacetate | 0.34 |
| Oxymorphone | 16.65 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

Begin by: (I) Water was added into a stainless steel vessel. (II) Opadry was added while stirring with a propeller mixer until all ingredients are finely dispersed in a suspension. (III) Oxymorphone and sucrose octacetate was added to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 45 mg/cm² to about 80 mg/cm² of the coat surrounding the coated tablet.

Representative results are shown in FIGS. 5b and 9.

### Example 6

### Oxycodone and Acetaminophen Sustained Action (SA) 30/325 mg Tablets

### (30/325 mg Tablets Contain 25 mg Maintenance Dose and 5 mg Loading Dose of Oxycodone and 325 mg of Acetaminophen)

### Formula for Maintenance Dose

| Ingredients | % w/w |
|---|---|
| Oxycodone | 6.25 |
| (particle size <400 | |
| microns) | |
| Polyethylene Oxide | 90.00 |
| (particle size <400 | |
| microns) | |
| Lactose Anhydrous DT | 5.25 |
| Crospovidone | 2.00 |
| Eudragit RL | 5.00 |
| Magnesium stearate | 0.50 |

### Formula for Loading Dose

| Ingredients | % w/w |
|---|---|
| Oxycodone (particle size | 1.25 |
| <400 microns) | |
| Acetaminophen (particle | 81.25 |
| size <400 microns) | |
| Hydroxypropyl | 4.00 |
| methylcellulose | |
| Crospovidone | 5.00 |
| Microcrystalline cellulose | 7.50 |
| Stearic Acid | 1.00 |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 100 mg/cm² of the coat surrounding the bi-layer tablet.

### Example 7

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle size | 5.00 |
| <400 microns) | |
| Polyethylene Oxide | 76.00 |
| (particle size <400 | |
| microns) | |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 75 00 |
| Oxycodone | 25.00 |
| (particle size | |
| <400 microns) | |
| water | qs |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone was added to the Opadry water mixture while stirring using a propeller mixer.

Step 4. Application of coating suspension from Step 3 to form part of the loading dose surrounding the tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension is applied to form about 40 mg/cm² to about 80 mg/cm² of the coat surrounding the coated tablet.

Representative results are shown in FIGS. 5a, 7, 8, 10, 11 and 12.

### Example 8

### Zolpidem Sustained Action (SA) 15 mg Tablets

### (15 mg Tablets Contain 15 mg Maintenance Dose and 5 mg Loading Dose)

### Formula for Maintenance Dose

| Ingredients | % w/w |
|---|---|
| Zolpidem (particle size | 5.00 |
| <500 microns) | |
| Polyethylene Oxide | 76.00 |
| (particle size <600 | |
| microns) | |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 75.00 |
| Crospovidone | 5.00 |
| Zolpidem | 20.00 |
| (particle size | |
| <500 microns) | |
| Water | qs |

### Formula for the Pod-Like Envelope

| Ingredients | % w/w |
|---|---|
| Eudragit E | 57.29 |
| Sodium Lauryl sulfate | 5.93 |
| Stearic acid | 8.89 |
| Crospovidone | 2.00 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hats rotary tablet press was used).

Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients are finely dispersed in a suspension. (III) Zolpidem was added to the Opadry water mixture while stirring using a propeller mixer.
Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

Step 5. Preparation of a Coating Suspension of the Ingredients for a Pod-Like Envelope was Applied to the Coated Tablet from Step 4:

(I) Water was added into a stainless steel vessel followed by sodium lauryl sulfate and stearic acid, step-by-step while stirring vigorously with a high shear mixer until all ingredients were dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 20 mg/cm² to about 35 mg/cm² of the coat surrounding the coated tablet.

### Example 9

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle size | 5.00 |
| <400 microns) | |
| Polyethylene Oxide | 76.50 |
| (particle size <400 | |
| microns) | |
| Lactose | 6.00 |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone | 25.00 |
| (particle size | |
| <400 microns) | |
| Water | qs |

### Formula for the Pod-Like Envelope

| Ingredients | % w/w |
|---|---|
| Eudragit E | 50.29 |
| Polacrilin Potassium | 9.00 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2 Preparation of Tablets Containing Maintenance Dose:

A rotary press was set-up to produce tablets (The Hata rotary tablet press was used). Granules from Step 1 were discharged into the feed hopper and compressed to form tablets.

### Step 3 Preparation of Coating Suspension of the Ingredients for the Loading Dose Applied on the Tablet:

(I) Water was added into a stainless steel vessel. (II) Opadry was added while stirring with a propeller mixer until all ingredients are finely dispersed in a suspension. (III) Oxycodone was added to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients were dissolved (II) Eudragit E was added step-by-step while stirring vigorously with a high shear mixer until all ingredients were dissolved. (III) Talc was added followed by Polacrilin potassium and simethicone while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension is applied to form about 15 mg/cm² to about 20 mg/cm² of the coat surrounding the coated tablet.

### Example 10

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle | 5.00 |
| size <400 microns) | |
| Polyethylene Oxide | 76.50 |
| (particle size <400 | |
| microns) | |
| Lactose | 6.00 |
| Crospovidone | 100 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle | 25.00 |
| size <400 microns) | |
| Water | qs |

### Formula for the Pod-Like Envelope

| Ingredients | % w/w |
|---|---|
| Polyvinyl alcohol | 46.73 |
| Polacrilin Potassium | 44.80 |
| Polethylene glycol | 4.67 |
| Talc | 1.90 |
| Titanium dioxide | 1.90 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients are finely dispersed in a suspension. (III) Oxycodone was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Hot water was added into a stainless steel vessel followed by polyethylene glycol, step-by-step, while stirring vigorously with a high shear mixer until dissolved. (II) Polyvinyl alcohol was added, step-by-step, while stirring vigorously with a high shear mixer until all ingredients were dissolved. (III) Polacrilin potassium was added followed by Talc and titanium dioxide while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/cm² to about 40 mg/cm² of the coat surrounding the coated tablet.

### Example 11

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle | 5.00 |
| size <400 microns) | |
| Polyethylene Oxide | 82.50 |
| (particle size <400 | |
| microns) | |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle | 25.00 |
| size <400 microns) | |
| Water | qs |

### Formula for the Pod-Like Envelope

| Ingredients | % w/w |
|---|---|
| Eudragit E | 4.67 |
| Polyvinyl alcohol | 40.19 |
| Polacrilin Potassium | 40.00 |
| Sodium lauryl sulfate | 4.67 |
| Stearic acid | 1.00 |
| Talc | 4.80 |
| Crospovidone | 4.67 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients are finely dispersed in a suspension. (III) Oxycodon was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cots Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of Coating Suspension of the Ingredients for a Pod-Like Envelope:

(I) Hot water was added into a stainless steel vessel followed by Sodium lauryl sulfate, Stearic acid, Eudragit E and Talc, step-by-step while stirring vigorously with a high shear mixer until dissolved. (II) Polyvinyl alcohol was added step-by-step while stirring vigorously with a high shear mixer until all ingredients were dissolved. (III) Polacrilin potassium was added followed by crospovidone while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/cm² to about 40 mg/cm² of the coat surrounding the coated tablet.

### Example 12

### Oxycodone Sustained Action (SA) 40 mg Tablets

### (40 mg Tablets Contain 35 mg Maintenance Dose and 6 mg Loading Dose)

### Formula for Maintenance Dose

| Ingredients | % w/w |
|---|---|
| Oxycodone (particle | 6.5 |
| size <400 microns) | |
| Polyethylene Oxide | 81.00 |
| (particle size <400 | |
| microns) | |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Oxycodone (particle | 2.50 |
| size <400 microns) | |
| Lactose | 70.00 |
| Hydroxypropyl | 4.00 |
| methylcellulose | |
| Crospovidone | 5.00 |
| Microcrystalline cellulose | 17.50 |
| Stearic acid | 1.00 |

### Formula for the Pod-Like Envelope

| Ingredients | % w/w |
|---|---|
| Eudragit E | 42.63 |
| Polyvinyl acetate | 10 |
| Talc | 22.10 |
| Magnesium stearate | 3.23 |
| Titanium dioxide | 18.94 |
| Polyethylene glycol 6000 | 3.10 |
| Water | qs |
| Isopropyl alcohol | qs |

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Polyethylene glycol was dissolved in water. This solution was added to isopropyl alcohol. (II) Eudragit E was added followed by Polyvinyl acetate, step-by-step, while stirring vigorously with a high shear mixer until all ingredients were dissolved. (III) Talc, titanium dioxide and magnesium stearate was added while stirring using a high shear mixer until finely dispersed in the solution.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension is applied to form about 10 mg/cm² to about 100 mg/cm² of the coat surrounding the bi-layer tablet.

### Example 13

### Oxycodone Sustained Action (SA) 40 mg Tablets

(40 mg Tablets contain 35 mg maintenance dose and 5 mg loading dose)

### Formula for Maintenance Dose

| Ingredients | % w/w |
|---|---|
| Oxycodone (particle | 5.00 |
| size <400 microns) | |
| Polyethylene Oxide | 76.50 |
| (particle size <400 | |
| microns) | |
| Lactose | 6.00 |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle | 25.00 |
| size <400 microns) | |
| Water | qs |

### Formula for the Pod-Like Envelope

| Ingredients | % w/w |
|---|---|
| Eudragit E | 36.09 |
| Polysaccharide | 12.00 |
| Sodium Lauryl sulfate | 5.93 |
| Stearic acid | 8.89 |
| Talc | 20.00 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Hot water was added into a stainless steel vessel followed by Sodium lauryl sulfate and Stearic acid, step-by-step, while stirring vigorously with a high shear mixer until dissolved. (II) Eudragit E was added followed by Talc, step-by-step, while stirring vigorously with a high shear mixer until all ingredients were dissolved. (III) Polyssacharide was added, followed by simethicone while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/1 cm² to about 40 mg/cm² of the coat surrounding the coated tablet.

### Example 14

### Oxycodone Sustained Action (SA) 40 mg Tablets

### (40 mg Tablets Contain 35 mg Maintenance Dose and 6 mg Loading Dose)

### Formula for Maintenance Dose

| Ingredients | % w/w |
|---|---|
| Oxycodone (particle | 6.5 |
| size < 400 microns) | |
| Polyethylene Oxide | 70.00 |
| (particle size <400 | |
| microns) | |
| Polacrilin Potassium | 11.00 |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Oxycodone (particle | 2.50 |
| size <400 microns) | |
| Polacrilin Potassium | 2.50 |
| Lactose | 67.50 |
| Hydroxypropyl | 4.00 |
| methylcellulose | |
| Crospovidone | 5.00 |
| Microcrystalline cellulose | 17.50 |
| Stearic acid | 1.00 |

### Formula for the Pod-Like Envelope

| Ingredients | % w/w |
|---|---|
| Eudragit E | 35.34 |
| Polysaccharide | 12.00 |
| Sodium Lauryl sulfate | 5.93 |
| Stearic acid | 8.89 |
| Talc | 20.75 |
| Simethicone | 17.09 |
| Water | qs |

### Processing Techniques

Step 1a. Preparation of Granules for the Maintenance Dose:

An Oxycodone-Polacrilin complex was prepared by continuously stirring Oxycodone and Polacrilin in water for 12 hours followed by filtration and drying of the complex such that less than 10% water is present. The dried complex and all the other ingredients with the exception of the magnesium stearate from the maintenance dose formula were charged into a high shear granulator and dry mix for less than 10 minutes. The granules were discharged into a Paterson Kelly V-Blender. The magnesium stearate was added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of a Coating Suspension of the Ingredients for a Pod-Like Envelope was Applied on the Bi-Layer Tablet:

(I) Water was added into a stainless steel vessel followed by Sodium lauryl sulfate and Stearic acid, step-by-step, while stirring vigorously with a high shear mixer until dissolved. (II) Eudragit E was added followed by Talc, step-by-step, while stirring vigorously with a high shear mixer until all ingredients were dissolved. (III) Polyssacharide was added followed by simethicone while stirring using a high shear mixer until finely dispersed in the solution.

Step 4. Application of a Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension is applied to form about 10 mg/cm² to about 60 mg/cm² of the coat surrounding the bi-layer tablet.

### Example 16

### Oxycodone Sustained Action (SA) 40 mg Tablets

### (40 mg Tablets Contain 35 mg Maintenance Dose and 6 mg Loading Dose)

### Formula for Maintenance Dose

| Ingredients | % w/w |
|---|---|
| Oxycodone (particle | 6.5 |
| size <400 microns) | |
| Polyethylene Oxide | 70.00 |
| (particle size <400 | |
| microns) | |
| Polacrilin Potassium | 11.00 |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 70.00 |
| Polacrilin Potassium | 5.00 |
| Oxycodone (particle | 25.00 |
| size <400 microns) | |
| Water | qs |

### Formula for the Pod-Like Envelope

| Ingredients | % w/w |
|---|---|
| Eudragit E | 38.09 |
| Polysaccharide | 10.00 |
| Sodium Lauryl sulfate | 5.93 |
| Stearic acid | 8.89 |
| Talc | 20.00 |
| Simethicorie | 17.09 |
| Water | qs |

### Processing Techniques

### Step 1. Preparation of Granules for the Maintenance Dose:

An Oxycodone-Polacrilin complex was prepared by continuously stirring Oxycodone and Polacrilin in water for 12 to 24 hours followed by filtration and drying of the complex such that less than 10% water is present. The dried complex and all the other Ingredients with the exception of the magnesium stearate from the maintenance dose formula were charged into a high shear granulator and dry mix for less than 10 minutes. The granules were discharged into a Paterson Kelly V-Blender. The magnesium stearate was added to the V-Blender. The granules were blended for less than 10 minutes.

### Step 2. Preparation of Tablets Containing Maintenance Dose:

A rotary press was set-up to produce tablets (The Hats rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone HCl was added, followed by Polacrilin to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and Stearic acid, step-by-step, while stirring vigorously with a high shear mixer until dissolved. (II) Eudragit E was added, followed by Talc, step-by-step, while stirring vigorously with a high shear mixer until all ingredients were dissolved. (III) Polyssacharide was added followed by simethicone while stirring using a high shear mixer until finely dispersed in the solution.

Step 6. Application of a Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 15 mg/cm² to about 35 mg/cm² of the coat surrounding the coated tablet.

### Example 16

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle | 6.5 |
| size < 400 microns) | |
| Polyethylene Oxide | 70.00 |
| (particle size <400 | |
| microns) | |
| Polacrilin Potassium | 11.00 |
| Crosoovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle | 25.00 |
| size <400 microns) | |
| Water | qs |

### Formula for the Pod-Like Envelope

| Ingredients | % w/w |
|---|---|
| Eudregit E | 41.27 |
| Polysaccharide | 12.00 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

An Oxycodone-Polacrilin complex was prepared by continuously stirring Oxycodone and Polacrilin in water for 12 to 24 hours followed by filtration and drying of the complex such that less than 10% water is present. The dried complex and all the other ingredients with the exception of the magnesium stearate from the maintenance dose formula were charged into a high shear granulator and dry mix for less than 10 minutes. The granules were discharged into a Paterson Kelly V-Blender. The magnesium stearate was added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone HCl was added, followed by Polacrilin to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and Stearic acid, step-by-step, while stirring vigorously with a high shear mixer until dissolved. (II) Eudragit E was added, followed by Talc, step-by-step, while stirring vigorously with a high shear mixer until all ingredients was dissolved. (III) Polyssacharide was added followed by simethicone while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 30 mg/cm² to about 60 mg/cm² of the coat surrounding the coated tablet.

### Example 17

### Morphine Sustained Action (SA) 30 mg Tablets

(30 mg Tablets contain 25 mg maintenance dose and 5 mg loading dose)

### Formula for Maintenance Dose

| Ingredients | % w/w |
|---|---|
| Morphine | 5.00 |
| (particle size 1000 | |
| microns | |
| Polyethylene Oxide | 57.00 |
| (particle size 1000 | |
| microns) | |
| Crospovidone | 5.00 |
| Microcrystalline cellulose | 5.00 |
| Eudragit RL | 5.00 |
| Triethy citrate | 10.00 |
| Capsicum oleoresin | 5.00 |
| Magnesium stearate | 1.00 |

### Formula for Loading Dose

| Ingredients | % w/w |
|---|---|
| Opadry | 83.00 |
| Capsicum oleoresin | 3.35 |
| Morphine | 16.65 |
| Water | qs |

### Formula for the Pod-Like Envelope

| Ingredigrents | % w/w |
|---|---|
| Eudragit E (milled) | 42.29 |
| Sodium Lauryl sulfate | 5.93 |
| Stearic acid (milled) | 8.89 |
| Talc | 25.89 |
| Simethicone | 17.00 |
| Water | qs |

### Processing Techniques

### Step 1. Preparation of Granules for the Maintenance Dose by Hot Melt Extrusion:

All the ingredients with the exception of the magnesium stearate and microcrystalline cellulose from the maintenance dose formula were added into a high shear granulator and dry mixed for less than 10 minutes. The dry mixed granules were discharged into a hopper of a Hot Melt Extruder and gradually fed into the Hot Melt Extruder heated barrel, while mixing by using the rotating screw element of the extruder. The material was extruded through a die attached at the end of a barrel. The extrudates were milled into granules. The milled granules were charged into a Paterson Kelly V-Blender. The magnesium stearate and microcrystalline cellulose were added into the V-Blender and blended for less than 10 minutes.

### Step 2. Preparation of Tablets Containing Maintenance Dose:

A rotary press was set-up to produce tablets (The Hata rotary tablet press was used).

Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Morphine and Capsicum oleoresin was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc was added, followed by simethicone while stirring using a high shear mixer until finely dispersed in the solution.

Step 6. Application of a Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 40 mg/cm² to about 50 mg/cm² of the coat surrounding the two-layered tablet.

### Example 18

### Oxycodone Sustained Action (SA) 30 mg Tablets

### (25 mg Tablets Contain 25 mg Maintenance Dose and 8 mg Loading Dose)

### Formula for Maintenance Dose

| Ingredients | % w/w |
|---|---|
| Oxycodone HC (particle | 5 |
| size <500 microns) | |
| Polyethylene Oxide | 66.00 |
| (particle size <600 | |
| microns) | |
| Polacrilin Potassium | 10.00 |
| Lactose | 6.00 |
| Crospovidone | 2.00 |
| Microcrystalline cellulose | 5.00 |
| Eudragit RL | 5.00 |
| Magnesium stearate | 0.50 |
| Water | qs |

### Formula for Loading Dose

| Ingredients | % w/w |
|---|---|
| Opadry | 75.00 |
| Crospovidone | 5.00 |
| Oxycodene HCl | 20.00 |
| (particle | |
| size <500 microns) | |
| Water | qs |

### Formula for the Pod-Like Envelope

| Ingredients | % w/w |
|---|---|
| Eudragit E | 57.29 |
| Sodium Lauryl sulfate | 5.93 |
| Stearic Acid | 8.89 |
| Crospovidone | 2.00 |
| Talc | 20.75 |
| Simethicone | 17.09 |
| Water | qs |

### Processing Techniques

### Step 1. Preparation of Granules for the Maintenance Dose:

An Oxycodone-Polacrilin complex was prepared by continuously stirring Oxycodone and Polacrilin in water for 12 to 24 hours followed by filtration and drying of the complex such that less than 10% water is present. The dried complex and all the other ingredients with the exception of the magnesium stearate from the maintenance dose formula were charged into a high shear granulator and dry mix for less than 10 minutes. The granules were discharged into a Paterson Kelly V-Blender. The magnesium stearate was added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone HCl was added to the Opadry water mixture while stirring using a propeller mixer.

Step 4 Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid step-by-step while stirring vigorously with a high shear mixer until all ingredients were dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 20 mg/cm² to about 35 mg/cm² of the coat surrounding the coated tablet.

### Example 19

### Oxycodone Sustained Action (SA) Tablets

### (30 mg Tablets Contain 25 mg Maintenance Dose and 6 mg Loading Dose)

### Formula for Maintenance Dose

| Ingredients | % w/w |
|---|---|
| Oxycodone | 5.00 |
| Polyethylene Oxide | 76.00 |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 83.00 |
| Sucrose Octeacetate | 0.34 |
| Oxycodone | 16.65 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2 Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone and sucrose octacetate was added to the opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients were dissolved. (II) Eudragit E was added, step-by-step, while stirring vigorously with a high shear mixer until all ingredients were dissolved. (III) Talc and simethicone were added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cola Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 45 mg/cm² to about 80 mg/cm² of the coat surrounding the coated tablet.

### Example 20

### Hydromorphone Sustained Action (SA) Tablets

### (16 mg Tablets Contain 12 mg Maintenance Dose and 4 mg Loading Dose)

### Formula for Maintenance Dose

| Ingredients | % w/w |
|---|---|
| Hydromorphone | 1.00 |
| Polyethylene Oxide | 56.00 |
| Lactose | 26.00 |
| Crospovidone | 2.00 |
| Microcrystalline cellulose | 9.50 |
| Eudragit RL | 5.00 |
| Magnesium stearate | 0.50 |

### Formula for Loading Dose

| Ingredients | % w/w |
|---|---|
| Opadry | 87.50 |
| Hydromorphone | 12.50 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Hydromorphone was added to the opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients are dissolved. (III) Talc and simethicone was added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 15 mg/cm² to about 55 mg/cm² of the coat surrounding the coated tablet.

### Example 21

### Hydromorphone Sustained Action (SA) 8 mg Tablets

### (8 mg Tablets Contain 7 mg Maintenance Dose and 1 mg Loading Dose)

### Formula for Maintenance Dose

| Ingredients | % w/w |
|---|---|
| Hydromorphone | 2.00 |
| (particle size 600 microns) | |
| Polyethylene Oxide | 40.00 |
| (particle size <600 | |
| microns) | |
| Lactose | 28.00 |
| Crospovidone | 5.00 |
| Fumaric Acid | 5.00 |
| Microcrystalline cellulose | 9.00 |
| Eudragit RL | 5.00 |
| Carbomer | 5.00 |
| Magnesium stearate | 1.00 |

### Formula for Loading Dose

| Ingredients | % w/w |
|---|---|
| Hydromorphone | 1.00 |
| (particle size 600 microns) | |
| Lactose | 60.00 |
| Fumaric Acid | 5.00 |
| Hydroxypropyl methyl | 4.00 |
| cellulose | |
| Pregelatinized starch | 5.00 |
| Microcrystalline cellulose | 20.00 |
| Magnesium stearate | 1.00 |

### Formula for the Pod-Like Envelope

| Ingredients | % w/w |
|---|---|
| Eudragit E | 74.50 |
| Citric Acid | 25.50 |
| Water | qs |

### Formula for the Overcoat

| Ingredients | % w/w |
|---|---|
| Opadry | 23.35 |
| Citric Acid | 76.65 |
| Water | qs |

### Processing Techniques

Step 1a. Preparation of Granules for the Maintenance Dose:

All the ingredients with exception of the magnesium stearate from the maintenance dose formula were charged into a high shear granulator and dry mixed for less than 10 minutes. The dry mixed materials were discharged into a Roll Compactor and the materials were forced between two counter rotating rolls in the Roll Compactor in order to form flakes or compacts. The compacts were granulated to reduce their size to uniform particle size distribution by passing them through a size reduction mill fitted with rotating blades and a perforated screen. The granules were discharged into a Paterson Kelly V-Blender. The magnesium stearate was added to the granules in the V-Blender and blend for less than 10 minutes.

Step 1b. Preparation of Granules for the Loading Dose:

All the ingredients, with the exception of the magnesium stearate and pregelatinized starch, from the loading dose formula were discharged into a high shear granulator and dry mixed for less than 10 minutes. The dry mixed materials were discharged into a Roll Compactor and the materials were forced between the two counter rotating rolls in the Roll Compactor in order to form flakes or compacts. The compacts were granulated to reduce their size to uniform particle size distribution by passing them through a size reduction mill fitted with rotating blades and perforated screen. The granules were discharged into a Paterson Kelly V-Blender. The magnesium stearate and pregelatinized starch were added to the granules in the V-Blender and blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of a Coating Suspension of the Ingredients of the Pod-Like Envelope Applied to the Bi-Layer Tablet:

(I) Water was added into a stainless steel vessel and Eudragit E was gradually added while stirring with a high shear mixer with controlled speed, sufficient to prevent sedimentation and lump formation. (II) The citric acid portion was added until a clear solution is obtained.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 10 mg/cm² to about 20 mg/cm² of the coat surrounding the bi-layer tablet.

Step 5. Preparation of Overcoating Suspension of the Ingredients of the Overcoat Applied to the Coated Tablet from Step 4:

(I) Water was added into a stainless steel vessel and Opadry was gradually added while stirring with a propeller mixer with controlled speed, sufficient to prevent sedimentation and lump formation. (II) The citric acid portion was added stepwise until no lumps was seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid per coated tablet.

### Example 22

### Morphine Sustained Action (SA) 30 mg Tablets

### (30 mg Tablets Contain 25 mg Maintenance Dose and 5 mg Loading Dose)

### Formula for Maintenance Dose

| Ingredients | % w/w |
|---|---|
| Morphine | 5.00 |
| (particle size 1000 | |
| microns) | |
| Polyethylene Oxide | 50.00 |
| (particle size 1000 | |
| microns) | |
| Crospovidone | 7.00 |
| Microcrystalline cellulose | 15.00 |
| Eudragit RL | 10.00 |
| Capsicum oleoresin | 5.00 |
| Magnesium stearate | 1.00 |

### Formula for Loading Dose

| Ingredients | % w/w |
|---|---|
| Opadry | 73.00 |
| Fumaric acid | 10 |
| Capsicum oleoresin | 3.35 |
| Morphine | 16.65 |
| Water | qs |

### Formula for the Pod-Like Envelope

| Ingredients | % w/w |
|---|---|
| Eudragit E (milled) | 59.29 |
| Sodium Lauryl sulfate | 5.93 |
| Stearic acid (milled) | 8.89 |
| Talc | 25.89 |
| Water | qs |

### Formula for the Overcoat

| Ingredients | % w/w |
|---|---|
| Opadry | 23.35 |
| Citric Acid | 76.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hate rotary tablet press was used). Granules from Step 1 were discharged into the feed hopper and compressed to form tablets.

### Step 3. Preparation of a Coating Suspension of the Ingredients for the Loading Dose was Applied to the Tablet:

(I) Water was added into a stainless steel vessel. (II) Opadry was added while stirring with a propeller mixer until all ingredients are finely dispersed in a suspension. (III) Morphine and Capsicum oleoresin was added to the Opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

Step 5. Preparation of a Coating Suspension of the Ingredients for a Pod-Like Envelope was Applied to the Coated Tablet from Step 4:

(I) Water was added to a stainless steel vessel, followed by Sodium lauryl sulfate and stearic acid, step-by-step, while stirring vigorously with a high shear mixer until all ingredients are dissolved. (II) Eudragit E was added step-by-step while stirring vigorously with a high shear mixer until all ingredients were dissolved. (III) Talc was added while stirring using a high shear mixer until finely dispersed in the solution.

Step 6. Application of the Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 20 mg/cm² to about 50 mg/cm² of the coat surrounding the coated tablet.

Step 7. Preparation of Overcoating Suspension of the Ingredients of the Overcoat Applied to the Coated Tablet from Step 6:

(I) Water and Isopropyl alcohol was added into a stainless steel vessel and citric acid was gradually added while stirring with a propeller mixer with controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid per coated tablet.

### Example 23

### Codeine Sustained Action (SA) 30 mg Tablets

### (30 mg Tablets Contain 25 mg Maintenance Dose and 5 mg Loading Dose)

### Formula for Maintenance Dose

| Ingredients | % w/w |
|---|---|
| Codeine | 5.00 |
| (particle size 600 microns) | |
| Polyethylene Oxide | 40.00 |
| (particle size <600 | |
| microns) | |
| Lactose | 25.00 |
| Crospovidone | 4.00 |
| Pre-gelatinized starch | 5.00 |
| Microcrystalline cellulose | 15.00 |
| Eudragit RL | 5.00 |
| Magnesium stearate | 1.00 |

### Formula for Loading Dose

| Ingredients | % w/w |
|---|---|
| Codeine | 2.50 |
| (particle size 600 microns) | |
| Lactose | 37.00 |
| Hydroxypropyl | 2.00 |
| methylcellulose | |
| Crospovidone | 7.50 |
| Citric Acid | 4.50 |
| Microcrystalline cellulose | 20.50 |
| Stearic acid | 1.50 |

### Formula for the Pod-Like Envelope

| Ingredients | % w/w |
|---|---|
| Eudragit E | 52.63 |
| Talc | 22.10 |
| Magnesium stearate | 3.23 |
| Titanium dioxide | 18.94 |
| Polyethylene glycol 6000 | 3.10 |
| Water | qs |
| Isopropyl alcohol | qs |

### Formula for the Overcoat

| Ingredients | % w/w |
|---|---|
| Opadry | 23.35 |
| Furnaric Acid | 76.65 |
| Water | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2 Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Isopropyl alcohol was added into a stainless steel vessel followed by Eudragit E, titanium dioxide, talc and magnesium stearate, step-by-step, while stirring vigorously with a high shear mixer until all ingredients were finely dispersed in a suspension. (II) Polyethylene glycol was dissolved in water. (III) The polyethylene glycol water mixture was added to the Eudragit E suspension while stirring using a high shear mixer.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 20 mg/cm² to about 30 mg/cm² of the coat surrounding the bi-layer tablet.

Step 5. Preparation of Overcoating Suspension of the Ingredients of the Overcoat Applied to the Coated Tablet from Step 4:

(I) Water was added into a stainless steel vessel and Opadry was gradually added while stirring with a propeller mixer with controlled speed, sufficient to prevent sedimentation and lump formation. (II) The fumaric acid portion was added stepwise until no lumps were seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of fumaric acid per coated tablet.

### Example 24

### Oxymorphone Sustained Action (SA) Tablets

### (30 mg Tablets Contain 26 mg Maintenance Dose and 5 mg Loading Dose)

### Formula for Maintenance Dose

| Ingredients | % w/w |
|---|---|
| Oxymorophone | 5.00 |
| Polyethylene Oxide | 56.00 |
| Lactose | 20.00 |
| Crospovidone | 3.00 |
| Microcrystalline cellulose | 10.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 83.00 |
| Sucrose Octaacetate | 0.34 |
| Oxymorphone | 16.65 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

Begin by: (I) Water was added into a stainless steel vessel. (II) Opadry was added while stirring with a propeller mixer until all ingredients are finely dispersed in a suspension. (III) Oxymorphone and sucrose octacetate was added to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 15 mg/cm² to about 80 mg/cm² of the coat surrounding the coated tablet.

Step 7. Preparation of Overcoating Suspension of the Ingredients of the Overcoat Applied to the Coated Tablet from Step 6:

(I) Water and Isopropyl alcohol was added into a stainless steel vessel and citric acid followed by fumaric acid was gradually added while stirring with a propeller mixer with controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 26

### Oxycodone and Acetaminophen Sustained Action (SA) 301325 mg

### Tablets (301326 mg Tablets Contain 25 mg Maintenance Dose and 5 mg Loading Dose of Oxycodone and 325 mg of Acetaminophen)

### Formula for Maintenance Dose

| Ingredients | % w/w |
|---|---|
| Oxycodone | 6.25 |
| (particle size <400 | |
| microns) | |
| Polyethylene Oxide | 50.00 |
| (particle size <400 | |
| microns) | |
| Lactose Anhydrous DT | 25.25 |
| Microcrystalline cellulose | 15.00 |
| Crospovidone | 5.00 |
| Eudragit RL | 7.00 |
| Magnesium stearate | 0.50 |

### Formula for Loading Dose

| Ingredients | % w/w |
|---|---|
| Oxycodone (particle size | 1.25 |
| <400 microns) | |
| Acetaminophen (particle | 81.25 |
| size <400 microns) | |
| Hydroxypropyl | 4.00 |
| methylcellulose | |
| Crospovidone | 5.00 |
| Microcrystalline cellulose | 7.50 |
| Stearic Acid | 1.00 |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 37.50 |
| Fumaric Acid | 62.50 |
| Isopropyl Alcohol | qs |
| Water | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 100 mg/cm² of the coat surrounding the bi-layer tablet.

Step 5. Preparation of Overcoating Suspension of the Ingredients of the Overcoat Applied to the Coated Tablet from Step 4:

(I) Water was added into a stainless steel vessel and Opadry was gradually added while stirring with a propeller mixer with controlled speed, sufficient to prevent sedimentation and lump formation. (II) The fumaric acid portion was added stepwise until no lumps were seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of fumaric acid per coated tablet.

### Example 26

### Oxycodone Sustained Action (SA) 40 mg Tablets

(40 mg Tablets contain 30 mg maintenance dose and 10 mg loading dose)

### Formula for Maintenance Dose

| Ingredients | % w/w |
|---|---|
| Oxycodone (particle size | 4.29 |
| <400 microns) | |
| Polyethylene Oxide | 70.71 |
| (particle size <400 | |
| microns) | |
| Lactose | 5.00 |
| Crospovidone | 3.00 |
| Microcrystalline cellulose | 11.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 84.20 |
| Oxycodone (particle size | 15.80 |
| <400 microns) | |
| water | qs |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.15 |
| Sucrose Octaacetate | 0.50 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

Step 3. Preparation of coating suspension of the ingredients for the loading dose was applied on the tablet:

(I) Water was added into a stainless steel vessel. (II) Opadry was added while stirring with a propeller mixer until all ingredients were finely dispersed in a suspension. (III) Oxycodone was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 40 mg/cm² to about 80 mg/cm² of the coat surrounding the coated tablet.
Step 7. Preparation of Overcoating Suspension of the Ingredients of the Overcoat Applied to the Coated Tablet from Step 6:

(I) Isopropyl alcohol was added into a stainless steel vessel. Sucroase Octaacetate was added followed by water while stirring. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 27

### Oxycodone and Acetaminophen Sustained Action (SA) 301325 mg

### Tablets (301325 mg Tablets Contain 25 mg Maintenance Dose and 5 mg Loading Dose of Oxycodone and 325 mg of Acetaminophen)

### Formula for Maintenance Dose

| Ingredients | % w/w |
|---|---|
| Oxycodone | 6.25 |
| (particle size <400 | |
| microns) | |
| Polyethylene Oxide | 50.00 |
| (particle size <400 | |
| microns) | |
| Lactose Anhydrous DT | 25.00 |
| Sucrose octaacetate | 0.25 |
| Microcrystalline cellulose | 15.00 |
| Crospovidone | 5.00 |
| Eudragit RL | 7.00 |
| Magnesium stearate | 0.50 |

### Formula for Loading Dose

| Ingredients | % w/w |
|---|---|
| Oxycodone (particle size | 1.25 |
| <400 microns) | |
| Acetaminophen (particle | 81.00 |
| size <400 microns) | |
| Sucrose Octaacetate | 0.25 |
| Hydroxypropyl | 4.00 |
| methylcellulose | |
| Crospovidone | 5.00 |
| Microcrystalline cellulose | 7.50 |
| Stearic Acid | 1.00 |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 37.50 |
| Furnaric Acid | 62.50 |
| Isopropyl Alcohol | qs |
| Water | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form from about 10 mg/cm² to about 100 mg/cm² of the coat surrounding the bi-layer tablet.

Step 5. Preparation of Overcoating Suspension of the Ingredients of the Overcoat Applied to the Coated Tablet from Step 4:

(I) Water was added into a stainless steel vessel and Opadry was gradually added while stirring with a propeller mixer with controlled speed, sufficient to prevent sedimentation and lump formation. (II) The fumaric acid portion was added stepwise until no lumps were seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of fumaric acid per coated tablet.

### Example 28

### Oxycodone Sustained Action (SA) 40 mg Tablets

### (40 mg Tablets Contain 30 mg Maintenance Dose and 10 mg Loading Dose)

### Formula for Maintenance Dose

| Ingredients | % w/w |
|---|---|
| Oxycodone (particle size | 4.29 |
| <400 microns) | |
| Polyethylene Oxide | 70.71 |
| (particle size <400 | |
| microns) | |
| Lactose | 5.00 |
| Crospovidone | 3.00 |
| Microcrystalline cellulose | 11.00 |
| Eudragit RL | 5.00 |
| Sucrose Octaacetate | 0.20 |
| Magnesium stearate | 0.50 |

### Formula for Loading Dose

| Ingredients | % w/w |
|---|---|
| Opadry | 84.20 |
| Oxycodone (particle size | 15.80 |
| <400 microns) | |
| Sucrose octaacetate | 0.20 |
| water | qs |

### Formula for the Pod-Like Envelope

| Ingredients | % w/w |
|---|---|
| Eudragit E | 59.00 |
| Sucrose Octaacetate | 0.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.15 |
| Sucrose Octaacetate | 0.50 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry followed by sucrose octaacetate was added while stirring with a propeller mixer until all ingredients were finely dispersed in a suspension. (III) Oxycodone was added to the Opadry/octaacetate water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 20 mg/cm² to about 60 mg/cm² of the coat surrounding the coated tablet.

Step 7. Preparation of Overcoating Suspension of the Ingredients of the Overcoat Applied to the Coated Tablet from Step 6:

(I) Isopropyl alcohol was added into a stainless steel vessel. Sucroase Octaacetate was added followed by water while stirring. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 29

### Oxycodone Sustained Action (SA) 40 mg Tablets

### (40 mg Tablets Contain 30 mg Maintenance Dose and 10 mg Loading Dose)

### Formula for Maintenance Dose

| Ingredients | % w/w |
|---|---|
| Oxycodone (particle size | 4.29 |
| <400 microns) | |
| Polyethylene Oxide | 50.71 |
| (particle size <400 | |
| microns) | |
| Lactose | 20.00 |
| Crospovidone | 6.00 |
| Microcrystalline cellulose | 13.50 |
| Eudragit RL | 5.00 |
| Sucrose Octaacetate | 0.00 |
| Magnesium stearate | 0.50 |

### Formula for Loading Dose

| Ingredients | % w/w |
|---|---|
| Opadry | 84.20 |
| Oxycodone (particle size | 15.80 |
| <400 microns) | |
| water | qs |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. A Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 8 mg/cm² to about 60 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8, Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 30

### Zolpidem Sustained Action (SA) 15 mg Tablets

### (15 mg Tablets Contain 16 mg Maintenance Dose and 5 mg Loading Dose)

### Formula for Maintenance Dose

| Ingredients | % w/w |
|---|---|
| Zolpidem (particle size | 5.00 |
| <500 microns) | |
| Polyethylene Oxide | 76.00 |
| (particle size <600 | |
| microns) | |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 75.00 |
| Crospovidone | 5.00 |
| Zolpidem (particle size | 20.00 |
| <500 microns) | |
| Water | qs |

### Formula for the Pod-Like Envelope

| Ingredients | % w/w |
|---|---|
| Eudragit E | 57.29 |
| Sodium Lauryl sulfate | 5.93 |
| Stearic acid | 8.89 |
| Crospovidone | 2.00 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hate rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients are finely dispersed in a suspension. (III) Zolpidem was added to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

Step 5. Preparation of a Coating Suspension of the Ingredients for a Pod-Like Envelope was Applied to the Coated Tablet from Step 4:

(I) Water was added into a stainless steel vessel followed by sodium lauryl sulfate and stearic acid, step-by-step while stirring vigorously with a high shear mixer until all ingredients were dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cola Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/cm² to about 35 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 31

### Oxycodone Sustained Action (SA) 40 mg Tablets

### (40 mg Tablets Contain 35 mg Maintenance Dose and 5 mg Loading Dose)

### Formula for Maintenance Dose

| Ingredients | % w/w |
|---|---|
| Oxycodone (particle size | 5.00 |
| <400 microns) | |
| Polyethylene Oxide | 46.50 |
| (particle size <400 | |
| microns) | |
| Sucrose octaacetate | 0.50 |
| Lactose | 26.00 |
| Crospovidone | 5.00 |
| Microcrystalline cellulose | 12.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle size | 25.00 |
| <400 microns) | |
| Water | qs |

### Formula for the Pod-Like Envelope

| Ingredients | % w/w |
|---|---|
| Eudragit E | 50.29 |
| Polacrilin Potassium | 9.00 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 76.60 |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose Applied on the Tablet:

(I) Water was added into a stainless steel vessel. (II) Opadry was added while stirring with a propeller mixer until all ingredients are finely dispersed in a suspension. (III) Oxycodone was added to the Opadry water mixture while stirring using a propeller mixer.

Step 4. Application of a Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients were dissolved (II) Eudragit E was added step-by-step while stirring vigorously with a high shear mixer until all ingredients were dissolved. (III) Talc was added followed by Polacrilin potassium and simethicone while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension is applied to form about 15 mg/cm² to about 20 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add fumaric acid while stirring with a propeller mixer under controlled speed, sufficient to prevent sedimentation and lump formation. (II) Add Opadry portion stepwise until no lumps are seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of fumaric acid per coated tablet.

### Example 32

### Pregabalin Sustained Action (SA) 333 mg Tablets

### (333 mg Tablets Contain 111 mg Maintenance Dose and 222 mg Loading Dose of Pregabalin)

### Formula for Maintenance Dose

| Ingredients | % w/w |
|---|---|
| Pregabalin | 37.00 |
| (particle size <400 | |
| microns) | |
| Polyethylene Oxide | 25.00 |
| (particle size <400 | |
| microns) | |
| Lactose Anhydrous DT | 16.40 |
| Microorystalline cellulose | 12.00 |
| Crospovidone | 2.00 |
| Eudragit RL | 7.00 |
| Magnesium stearate | 0.60 |

### Formula for Loading Dose

| Ingredients | % w/w |
|---|---|
| Pregabalin (particle size | 37.00 |
| <400 microns) | |
| Lactose | 22.41 |
| Hydroxypropyl | 5.00 |
| methylcellulose | |
| Crospovidone | 5.00 |
| Microcrystalline cellulose | 12.00 |
| Magnesium stearate | 0.5 |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 37.50 |
| Fumaric Acid | 62.50 |
| Isopropyl Alcohol | qs |
| Water | qs |

### Processing Techniques

Step 1a. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

Step 1b. Preparation of Granules for the Loading Dose:

All the ingredients with the exception of the stearic acid from the loading dose formula were charged into a high shear granulator and dry mixed for less than 10 minutes. The dry mixed granules were discharged into a Paterson Kelly V-Blender. The stearic acid was then added to the V-Blender. The granules were blended for less than 10 minutes.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of Coating Suspension of the Ingredients for a Pod-Like Envelope was Applied on the Bi-Layer Tablet:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 4. Application of Coating Suspension from Step 3 to Form a Pod-Like Envelope Surrounding the Bi-Layer Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form from about 10 mg/cm² to about 100 mg/cm² of the coat surrounding the bi-layer tablet.

Step 5. Preparation of Overcoating Suspension of the Ingredients of the Overcoat Applied to the Coated Tablet from Step 4:

(I) Water was added into a stainless steel vessel and Opadry was gradually added while stirring with a propeller mixer with controlled speed, sufficient to prevent sedimentation and lump formation. (II) The fumaric acid portion was added stepwise until no lumps were seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of fumaric acid per coated tablet.

### Example 33

### Amphetamine Sustained Action (SA) 20 mg Tablets

### (20 mg Tablets Contain 10 mg Maintenance Dose and 10 mg Loading Dose) Equivalent to a Total of 12.5 mg Amphetamine Base

### Formula for Maintenance Dose

| Ingredients | % w/w |
|---|---|
| Dextroamphetamine | 1.25 |
| Sulfate | |
| (particle size <400 | |
| microns) | |
| Amphetamine sulfate | 1.25 |
| (particle size <400 | |
| microns) | |
| Polyethylene Oxide | 50.71 |
| (particle size <400 | |
| microns) | |
| Lactose | 22.79 |
| Crospovidone | 5.00 |
| Microcrystalline cellulose | 13.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 49.75 |
| Sucrose octaacetate | 0.25 |
| Dextroamphetamine | 25.00 |
| Saccharate | |
| (particle size <400 | |
| microns) | |
| Amphetamine Aspartate | 25.00 |
| Monohydrate | |
| (particle size <400 | |
| microns) | |
| water | qs |

### Formula for the Pod-Like Envelope

| Ingredients | % w/w |
|---|---|
| Eudragit E | 50.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hats rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry and sucrose octaacetate were added while stirring with a propeller mixer until all ingredients were finely dispersed in a suspension. (III) Dextroamphetamine Saccharate and Amphetamine Aspartate Monohydrate was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high sheer mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 8 mg/cm² to about 60 mg/cm² of the coat surrounding the coated tablet.

Stem 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 34

### Amphetamine Sustained Action (SA) 20 mg Tablets

### (20 mg Tablets Contain 10 mg Maintenance Dose and 10 mg Loading Dose) Equivalent to a Total of 12.5 mg Amphetamine Base

### Formula for Maintenance Dose

| Ingredients | % w/w |
|---|---|
| Dextroamphetamine | 1.25 |
| Sulfate | |
| (particle size <400 | |
| microns) | |
| Amphetamine sulfate | 1.25 |
| (particle size <400 | |
| microns | |
| Polyethylene Oxide | 50.71 |
| (particle size <400 | |
| microns) | |
| Lactose | 22.79 |
| Crospovidone | 5.00 |
| Microcrystalline cellulose | 13.50 |
| Eudragit RL | 5.00 |
| Magnesium stearate | 0.50 |

### Formula for Loading Dose

| Ingredients | % w/w |
|---|---|
| Opadry | 50.00 |
| Dextroamphetamine | 25.00 |
| Saccharate | |
| (particle size <400 | |
| microns) | |
| Amphetamine Aspartate | 25.00 |
| Monohydrate | |
| (particle size <400 | |
| microns) | |
| water | qs |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.25 |
| Sucrose octaacetate | 0.50 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Dextroamphetamine Saccharate and Amphetamine Aspartate Monohydrate was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc, sucrose octaacetate and simethicone was added while stirring using a high shear mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cots Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 8 mg/cm² to about 60 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet and enough sucrose octaacetate to make the tablet objectionable to taste

### Example 35

### Amphetamine Sustained Action (SA) 20 mg Tablets

### (20 mg Tablets Contain 10 mg Maintenance Dose and 10 mg Loading Dose) Equivalent to a Total of 12.5 mg Amphetamine Base

### Formula for Maintenance Dose

| Ingredients | % w/w |
|---|---|
| Dextroamphetamine | 1.25 |
| Sulfate | |
| (particle size <400 | |
| microns) | |
| Amphetamine sulfate | 1.25 |
| (particle size <400 | |
| microns) | |
| Polyethylene Oxide | 50.71 |
| (particle size <400 | |
| microns) | |
| Lactose | 22.79 |
| Crospovidone | 5.00 |
| Miroccrystalline cellulose | 13.50 |
| Eudragit RL | 5.00 |
| Magnesium stearate | 0.50 |

### Formula for Loading Dose

| Ingredients | % w/w |
|---|---|
| Opadry | 50.00 |
| Dextroamphetamine | 25.00 |
| Saccharate | |
| (particle size <400 | |
| microns) | |
| Amphetamine Aspartate | 25.00 |
| Monohydrate | |
| (particle size <400 | |
| microns) | |
| water | qs |

### Formula for the Pod-Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hats rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Dextroamphetamine Saccharate and Amphetamine Aspartate Monohydrate was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension was applied to form a coat surrounding the tablet.

### Step 5. Preparation of a Coating Suspension of the Ingredients for a Pod-Like Envelope:

(I) Water was added into a stainless steel vessel followed by sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of a Coating Suspension from Step 5 to Form a Pod-Like Envelope Surrounding the Coated Tablet from Step 4:

Tablets from Step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan.

A sufficient amount of the suspension is applied to form about 8 mg/cm² to about 60 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 36

### Oxycodone Sustained Action (SA) 40 mg Tablets

### (40 mg Tablets Contain 36 mg Maintenance Dose and 6 mg Loading Dose)

### Formula for Maintenance Dose

| Ingredients | % w/w |
|---|---|
| Oxycodone (particle size | 5.00 |
| <400 microns) | |
| Polyethylene Oxide | 76.50 |
| (particle size <400 | |
| microns) | |
| Lactose | 6.00 |
| Crospovidone | 2.00 |
| Microcrystalline cellulose | 5.00 |
| Eudragit RL | 5.00 |
| Magnesium stearate | 0.50 |

### Formula for Loading Dose

| Ingredients | % w/w |
|---|---|
| Opadry | 75.00 |
| Oxycodone (particle size | 25.00 |
| <400 microns) | |
| Water | qs |

### Formula for the Pod-Like Envelope

| Ingredients | % w/w |
|---|---|
| Eudragit E | 36.09 |
| Polysaccharide | 12.00 |
| Sodium Lauryl sulfate | 5.93 |
| Stearic acid | 8.89 |
| Talc | 20.00 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Hot water was added into a stainless steel vessel followed by Sodium lauryl sulfate and Stearic acid, step-by-step, while stirring vigorously with a high shear mixer until dissolved. (II) Eudragit E was added followed by Talc, step-by-step, while stirring vigorously with a high shear mixer until all ingredients were dissolved. (III) Polyssacharide was added, followed by simethicone while stirring using a high shear mixer until finely dispersed in the solution.

Step 6. Application of Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/cm² to about 40 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 37

### Morphine Sustained Action (SA) 30 mg Tablets

### (30 mg Tablets Contain 25 mg Maintenance Dose and Mg Loading Dose)

### Formula for Maintenance Dose

| Ingredients | % w/w |
|---|---|
| Morphine | 5.00 |
| (particle size 1000 | |
| microns) | |
| Polyethylene Oxide | 57.00 |
| (particle size 1000 | |
| microns) | |
| Crospovidone | 5.00 |
| Microcrystalline cellulose | 5.00 |
| Eudragit RL | 5.00 |
| Triethy citrate | 10.00 |
| Capsicum oleoresin | 5.00 |
| Magnesium stearate | 1.00 |

### Formula for Loading Dose

| Ingredients | % w/w |
|---|---|
| Opadry | 83.00 |
| Capsicum oleoresin | 3.35 |
| Morphine | 16.65 |
| Water | qs |

### Formula for the Pod-Like Envelope

| Ingredients | % w/w |
|---|---|
| Eudragit E (milled) | 42.29 |
| Sodium Lauryl sulfate | 5.93 |
| Stearic acid (milled) | 8.89 |
| Talc | 25.89 |
| Simethicone | 17.00 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose by Hot Melt Extrusion:

All the ingredients with the exception of the magnesium stearate and microcrystalline cellulose from the maintenance dose formula were added into a high shear granulator and dry mixed for less than 10 minutes. The dry mixed granules were discharged into a hopper of a Hot Melt Extruder and gradually fed into the Hot Melt Extruder heated barrel, while mixing by using the rotating screw element of the extruder. The material was extruded through a die attached at the end of a barrel. The extrudates were milled into granules. The milled granules were charged into a Paterson Kelly V-Blender. The magnesium stearate and microcrystalline cellulose were added into the V-Blender and blended for less than 10 minutes.

### Step 2 Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Morphine and Capsicum oleoresin was added to the Opadry water mixture while stirring using a propeller mixer.

Step 4. Application of Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc was added, followed by slmethicone while stirring using a high shear mixer until finely dispersed in the solution.

Step 6. Application of a Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 40 mg/cm² to about 50 mg/cm² of the coat surrounding the two-layered tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 38

### Oxycodone Sustained Action (SA) 30 mg Tablets

### (25 mg Tablets Contain 25 mg Maintenance Dose and 6 mg Loading Dose)

### Formula for Maintenance Dose

| Ingredients | % w/w |
|---|---|
| Oxycodone HCl (particle | 5 |
| size <500 microns) | |
| Polyethylene Oxide | 66.00 |
| (particle size <600 | |
| microns) | |
| Polacrilin Potassium | 10.00 |
| Lactose | 6.00 |
| Crospovidone | 2.00 |
| Microcrystalline cellulose | 5.00 |
| Eudragit RL | 5.00 |
| Magnesium stearate | 0.50 |
| Water | qs |

### Formula for Loading Dose

| Ingredients | % w/w |
|---|---|
| Opacity | 75.00 |
| Crospovidone | 5.00 |
| Oxycodone HCl (particle | 20.00 |
| size <500 microns) | |
| Water | qs |

### Formula for the Pod-Like Envelope

| Ingredients | % w/w |
|---|---|
| Eudragit E | 57.29 |
| Sodium Lauryl sulfate | 5.93 |
| Stearic Acid | 8.89 |
| Crospovidone | 2.00 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

An Oxycodone-Polacrilin complex was prepared by continuously stirring Oxycodone and Polacrilin in water for 12 to 24 hours followed by filtration and drying of the complex such that less than 10% water is present. The dried complex and all the other ingredients with the exception of the magnesium stearate from the maintenance dose formula were charged into a high shear granulator and dry mix for less than 10 minutes. The granules were discharged into a Paterson Kelly V-Blender. The magnesium stearate was added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone HCl was added to the Opadry water mixture while stirring using a propeller mixer.

Step 4 Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

Step 5. Preparation of a coating suspension of the ingredients for a Pod-like envelope:

(I) Water was added into a stainless steel vessel followed by Sodium lauryl sulfate and stearic acid step-by-step while stirring vigorously with a high shear mixer until all ingredients were dissolved. (II) Eudragit E was added, step-by-step, while stirring vigorously with a high shear mixer until all ingredients were dissolved. (III) Talc and simethicone was added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 20 mg/cm² to about 35 mg/cm^{a} of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 39

### Oxycodone Sustained Action (SA) Tablets

### (30 mg Tablets Contain 28 mg Maintenance Dose and 6 mg Loading Dose)

### Formula for Maintenance Dose

| Ingredients | % w/w |
|---|---|
| Oxycodone | 5.00 |
| Polyethylene Oxide | 76.00 |
| Lactose | 6.00 |
| Crospovidone | 2.00 |
| Microcrystalline cellulose | 5.00 |
| Eudragit RL | 5.00 |
| Sucrose Octaacetate | 0.50 |
| Magnesium stearate | 0.50 |

### Formula for Loading Dose

| Ingredients | % w/w |
|---|---|
| Opadry | 83.00 |
| Sucrose Octaacetate | 0.34 |
| Oxycodone | 16.65 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose:

All the ingredients with the exception of the magnesium stearate from the maintenance dose formula were charged into a high shear granulator and dry mixed for less than 10 minutes. The dry mixed granules were discharged into a Paterson Kelly V-Blender. The magnesium stearate was then added to the V-Blender. The granules were blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Oxycodone and sucrose octacetate was added to the opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients were dissolved. (II) Eudragit E was added, step-by-step, while stirring vigorously with a high shear mixer until all ingredients were dissolved. (III) Talc and simethicone were added while stirring using a high shear mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 45 mgicm² to about 80 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 40

### Hydromorphone Sustained Action (SA) Tablets

### (16 mg Tablets Contain 12 mg Maintenance Dose and 4 mg Loading Dose)

### Formula for Maintenance Dose

| Ingredients | % w/w |
|---|---|
| Hydromorphone | 1.00 |
| Polyethylene Oxide | 6.00 |
| Lactose | 26.00 |
| Crospovidone | 2.00 |
| Microcrystalline cellulose | 9.50 |
| Eudragit RL | 5.00 |
| Magnesium stearate | 0.50 |

### Formula for Loading Dose

| Ingredients | % w/w |
|---|---|
| Opadry | 87.50 |
| Hydromorphone | 12.50 |
| Water | qs |

### Formula for the Pod Like Envelope

| Ingredients | % w/w |
|---|---|
| Eudragit E | 59.29 |
| Sodium Lauryl sulfate | 5.93 |
| Stearic acid | 8.89 |
| Talc | 20.75 |
| Simethicone | 17.09 |
| Water | qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | qs |
| Water | qs |

### Processing Techniques

### Step 1. Preparation of Granules for the Maintenance Dose by Hot Melt Extrusion:

All the ingredients with the exception of the magnesium stearate and microcrystalline cellulose from the maintenance dose formula were added into a high shear granulator and dry mixed for less than 10 minutes. The dry mixed granules were discharged into a hopper of a Hot Melt Extruder and gradually fed into the Hot Melt Extruder heated barrel, while mixing by using the rotating screw element of the extruder. The material was extruded through a die attached at the end of a barrel. The extrudates were milled into granules. The milled granules were charged into a Paterson Kelly V-Blender. The magnesium stearate and microcrystalline cellulose were added into the V-Blender and blended for less than 10 minutes.

### Step 2. Preparation of Tablets Containing Maintenance Dose:

A rotary press was set-up to produce tablets (The Hata rotary tablet press was used). Granules from Step 1 were discharged into the feed hopper and compressed to form tablets.

### Step 3. Preparation of a Coating Suspension of the Ingredients for the Loading Dose was Applied on the Tablet:

(I) Water was added into a stainless steel vessel. (II) Opadry was added while stirring with a propeller mixer until all ingredients were finely dispersed in a suspension. (III) Hydromorphone was added to the opadry water mixture while stirring using a propeller mixer.

Step 4. Application of the Coating Suspension from Step 3 to Form Part of the Loading Dose Surrounding the Tablet from Step 2:

Tablets from step 2 were charged into a rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. The suspension is applied to form a coat surrounding the tablet.

### Step 5. Preparation of a Coating Suspension of the Ingredients for a Pod-Like Envelope:

(I) Water was added into a stainless steel vessel followed by Sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients are dissolved. (III) Talc and simethicone was added while stirring using a high sheer mixer until finely dispersed in the solution.

Step 6. Application of the Coating Suspension from Step 5 to Form a Pod-Like Envelope Surrounding the Coated Tablet from Step 4:

Tablets from step 4 were charged into the rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied to form about 10 mg/cm² to about 55 mg/cm² of the coat surrounding the coated tablet.

Step 7. Preparation of Overcoating Suspension of the Ingredients of the Overcoat Applied to the Coated Tablet from Step 6:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 8. Application of Coating Suspension from Step 7 to Form an Overcoat Surrounding the Coated Tablets from Step 6:

Tablets from step 6 were charged into a rotating drum of a side vented automated Tablet coater (Rama Cota Tablet Film Coater was used). The suspension from Step 7 was applied to the tablets obtained from Step 6, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 41

### Morphine Sustained Action (SA) 30 mg Tablets

### (30 mg Tablets Contain 25 mg Maintenance Dose and 5 mg Loading Dose)

### Formula for Maintenance Dose

| Ingredients | % w/w |
|---|---|
| Morphine | 5.00 |
| (particle size 1000 | |
| microns) | |
| Polyethylene Oxide | 57.00 |
| (particle size 1000 | |
| microns) | |
| Crospovidone | 5.00 |
| Microcrystalline cellulose | 5.00 |
| Eudragit RL | 5.00 |
| Triethy citrate | 10.00 |
| Capsicum oleoresin | 5.00 |
| Magnesium stearate | 1.00 |

### Formula for Loading Dose

| Ingredients | % w/w |
|---|---|
| Soluplus | 54.00 |
| Cremaphor RH 40 | 6.00 |
| Capsicum oleoresin | 3.35 |
| Morphine | 16.65 |
| Microcrystalline cellulose | 9.00 |
| Magnesium Stearate | 1.00 |

### Formula for the Pod-Like Envelope

| Ingredients | % w/w |
|---|---|
| Eudragit E (milled) | 42.29 |
| Sodium Lauryl sulfate | 5.93 |
| Stearic acid (milled) | 8.89 |
| Talc | 25.89 |
| Simethicone | 17.00 |
| Water | Qs |

### Formula for the Overcoat

| Ingredients | % w/w |
|---|---|
| Opadry | 23.35 |
| Fumaric Acid | 38.00 |
| Citric Acid | 38.65 |
| Isopropyl Alcohol | Qs |
| Water | Qs |

### Processing Techniques

Step 1a. Preparation of Granules for the Maintenance Dose by Hot Melt Extrusion:

All the ingredients with the exception of the magnesium stearate and microcrystalline cellulose from the maintenance dose formula were added into a high shear granulator and dry mixed for less than 10 minutes. The dry mixed granules were discharged into a hopper of a Hot Melt Extruder and gradually fed into the Hot Melt Extruder heated barrel, while mixing by using the rotating screw element of the extruder. The material was extruded through a die attached at the end of a barrel. The extrudates were milled into granules. The milled granules were charged into a Paterson Kelly V-Blender. The magnesium stearate and microcrystalline cellulose were added into the V-Blender and blended for less than 10 minutes.

Step 1b. Preparation of the Granules for Loading Dose by Hot Melt Extrusion:

All the ingredients with the exception of the magnesium stearate and microcrystalline cellulose from the maintenance dose formula were added into a high shear granulator and dry mixed for less than 10 minutes. The dry mixed granules were discharged into a hopper of a Hot Melt Extruder and gradually fed into the Hot Melt Extruder heated barrel, while mixing by using the rotating screw element of the extruder. The material was extruded through a die attached at the end of a barrel. The extrudates were milled into granules. The milled granules were charged into a Paterson Kelly V-Blender. The magnesium stearate and microcrystalline cellulose were added into the V-Blender and blended for less than 10 minutes. The barrel section temperatures of the hot melt extruder are typically optimized so that the viscosity of the melt is low enough to allow conveying down the barrel and proper mixing, while keeping temperatures low enough to avoid thermal degradation of the materials; typically about 100 to about 200° C.

### Step 2. Preparation of a Bi-Layer Tablet Containing Maintenance Dose and Loading Dose:

The first layer is made from the granules prepared in Step 1a, and the second layer is made from granules prepared in Step 1b. A double rotary press was set-up to produce a bi-layer tablet (The Karnavati UNIK I FC double rotary and double layer tablet press was used). Granules from Step 1a were charged into a first feed hopper and granules from Step 1b were charged into a second feed hopper and the bi-layer tablet was produced from the double rotary press.

### Step 3. Preparation of a Coating Suspension of the Ingredients for a Pod-Like Envelope to be Applied to the Bi-Layer Tablet:

(I) Water was added into a stainless steel vessel followed by Sodium lauryl sulfate and stearic acid, step-by-step, while stirring vigorously with a high shear mixer until all ingredients are dissolved. (II) Eudragit E was added, step-by-step, while stirring vigorously with a high shear mixer until all ingredients were dissolved. (III) Talc was added, followed by simethicone while stirring using a high shear mixer until finely dispersed in the solution.

Step 4. Application of a Coating Suspension from Step 3 to Form a Pod-Like Envelope Surrounding the Bi-Layer Tablet from Step 2:

Tablets from step 2 were charged into the rotating drum of a side vented automated Tablet coater (Rama Cota Tablet Film Coater was used). The suspension from Step 3 was applied to the tablets obtained from Step 2, using a peristaltic pump and spray gun. The suspension was dried as a film onto the tablets, using heated air drawn through the tablet bed from an inlet fan. A sufficient amount of the suspension was applied to form about 10 mg/cm² to about 50 mg/cm² of the coat surrounding the bi-layer tablet.

Step 5. Preparation of Overcoating Suspension of the Ingredients of the Overcoat Applied to the Coated Tablet from Step 4:

(I) Isopropyl alcohol followed by water was added into a stainless steel vessel. Gradually add citric acid followed by fumaric acid while stirring with a propeller mixer under controlled speed, sufficient to prevent sedimentation and lump formation. (II) The Opadry portion was added stepwise until no lumps were seen.

Step 6. Application of Coating Suspension from Step 5 to Form an Overcoat Surrounding the Coated Tablets from Step 4:

Tablets from step 4 were charged into a rotating drum of a side vented automated Tablet coater (Rama Cota Tablet Film Coater was used). The suspension from Step 5 was applied to the tablets obtained from Step 4, using a peristaltic pump and spray gun. The suspension was dried as a film onto the tablets, using heated air drawn through the tablet bed from an inlet fan. A sufficient amount of the suspension was applied such that the coat contained from 10 mg to 600 mg of citric acid and fumaric acid per coated tablet.

### Example 42

### Results of Vaporization Studies

### Experimental Conditions

Some tablets, as noted in the Examples above, were milled and placed in a flat bottom flask. The flask was connected to a condenser via a 3-way connector. The condenser was sealed at the top with a cotton plug and connected to a vacuum pump to help pull the vapor produced by heating the bottom of the flask using a hot plate at 540° C.

Extraction of Oxycodone HCl (Active) after Subjecting Milled Oxycodone HCl Extended Release Tablets Described Above in the Examples to 6540 OC of Heat Usina a Hot Plate

| | Amount | Amount |
|---|---|---|
| | extracted | extracted |
| Description of source of vapor extract | Sample 1 | Sample 2 |
| Theoretical Amount of Active in milled | 38.18 | 38.16 |
| tablet before heating (mg) | | |
| Amount of Active extracted after heating | 0.57 | 0.52 |
| for 2 minutes in Cotton plug (mg) | | |
| Amount of Active extracted after heating | 0.30 | 0.29 |
| for 2 minutes in Condenser (mg) | 1.33 | 1.58 |
| Amount of Active extracted after heating | | |
| for 2 minutes in 3-Way Connector (mg) | | |
| Total Amount of Active recovered after | 2.20 | 2.39 |
| heatin for 2 minutes (mg) | | |
| Recovery (%) | 5.8 | 6.3 |

| | |
|---|---|
| Mean Recover (%)* | 6.0 |

*Material not completely burnt

| | Amount | Amount | Amount |
|---|---|---|---|
| | extracted | extracted | extracted |
| Description of source of vapor extract | Sample 1 | Sample 2 | Sample 3 |
| Theoretical Amount of Active in milled | 38.16 | 38.16 | 38.16 |
| tablet before heating (mg) | | | |
| Amount of Active extracted after heating | 0.40 | 0.32 | 0.54 |
| for 6 minutes in Cotton plug (mg) | | | |
| Amount of Active extracted after heating | 1.84 | 1.57 | 2.33 |
| for 5 minutes in Condenser (mg) | | | |
| Amount of Active extracted after heating | 3.00 | 3.38 | 2.60 |
| for 6 minutes in 3-Way Connector (mg) | | | |
| Total Amount of Active recovered after | 5.23 | 5.27 | 5.46 |
| heating for 5 minutes | | | |
| Recovery (%) | 13.7 | 13.8 | 14.3 |

| | |
|---|---|
| Mean Recovery %** | 13.9 |

**Material completely burnt

We claim:

1. A formulation comprising:
at least one primary active substance, wherein onset of action of said at least one primary active substance is potentiated by the presence of a loading dose comprising said at least one primary active substance; and
at least one coat comprising Eudragit E (dimethylaminoethyl methacrylate copolymer),
wherein the formulation is free of any active substance external to said at least one coat.

2. The formulation according to claim 1, wherein the release of said at least one primary active substance shows a Point Of Divergence (POD), in a dissolution profile, with the loading dose representing a point in a timeline where the history of the dissolution or release rate changes from an onset of action to another set of points in the timeline represented by a controlled release.

3. The formulation according to claim 1, wherein prior to the POD, there is no significant controlled release.

4. The formulation according to claim 1, wherein the formulation further comprises at least one secondary active substance, wherein the formulation has a quick onset of action of said at least one primary active substance followed by a controlled release of at least one secondary active substance, or vice versa, wherein said at least one primary active substance and said at least one secondary active substance are the same or different.

5. The formulation according to claim 1, wherein the formulation further comprises at least one secondary active substance, wherein said at least one primary active substance in the loading dose is released at a higher rate in comparison to another dose having said at least one secondary active substance, wherein said at least one primary active substance and said at least one secondary active substance are the same or different.

6. The formulation according to claim 1 further comprising a core, wherein said at least one primary active substance is incorporated into the core, external to the core, or a combination thereof.

7. The formulation according to claim 1, wherein the formulation further comprises a maintenance dose having at least one secondary active substance, wherein said at least one secondary active substance is the same or different than said at least one primary active substance.

8. The formulation according to claim 7, wherein the maintenance dose comprises said at least one secondary active substance in a controlled release matrix.

9. The formulation according to claim 8, wherein the formulation comprises at least one layer of said at least one loading dose and at least one layer of said at least one maintenance dose.

10. The formulation according to claim 9, wherein said at least one layer of said at least one loading dose covers at least a portion of said at least one layer of said at least one maintenance dose or vice versa, forming a layered formulation.

11. The formulation according to claim 10, wherein said at least one coat surrounds said layered formulation.

12. The formulation according to claim 8 further comprises a core having said at least one maintenance dose and at least one coat comprising said at least one loading dose.

13. The formulation according to claim 12 further comprises at least one coat comprising at least one maintenance dose, which said at least one maintenance dose in the core is the same or different than said maintenance dose in said at least one coat.

14. The formulation according to claim 13, wherein the core further comprises said at least one loading dose and/or said at least one coat of said loading dose further comprises said at least one maintenance dose, which said at least one loading dose in the core is the same or different than said loading dose in said at least one coat.

15. The formulation according to claim 12, wherein said at least one coat comprising said at least one loading dose significantly covers said core.

16. The formulation according to claim 8 further comprises a core, wherein said at least one loading dose and said at least one maintenance dose are external to the core.

17. The formulation according to claim 1, wherein said at least one coat comprising the Eudragit E further comprises at least one active substance, wherein said at least one active substance and said at least one primary active substance are the same or different.

18. The formulation according to claim 1, wherein said at least one coat controls the release of said at least one primary active substance.

19. The formulation according to claim 18, wherein release of any active substance in the formulation is activated by a pH dependent mechanism, ion-exchange dependent mechanism, bacterial flora/enzymes dependent mechanism, or a combination thereof.

20. The formulation according to claim 19, wherein the pH for the pH dependent mechanism is at most about 5.

21. The formulation according to claim 19, wherein the ion-exchange mechanism is controlled by at least one ion-exchange resin.

22. The formulation according to claim 21, wherein said at least one ion-exchange resin is selected from Cholestyramine, Colestipol, Sodium polystyrene sulfonate, Polacrilex resin, and/or Polacrilin potassium.

23. The formulation according to claim 19, wherein the bacterial flora/enzymes dependent mechanism is controlled by at least one polymer reactive to intestinal bacterial flora/enzymes.

24. The formulation according to claim 23, wherein said at least polymer is selected from polysaccharides such as guar gum, inulin, chondrotin sulphate, alginates, and/or dextran.

25. The formulation according to claim 1, wherein the Eudragit E comprises Eudragit E 100™.

26. The formulation according to claim 1, wherein up to about 55% of the total dose is released as a loading dose to manage pain.

27. The formulation according to claim 26, wherein the loading dose is released within about 60 minutes of ingestion.

28. The formulation according to claim 1, wherein the formulation is configured such that when the formulation is administered in a physically compromised form to a subject, the rate of release of said at least one primary active substance in the loading dose is substantially the same or lower than the rate of release of said at least one primary active substance in the loading dose when the formulation is administered in an intact form.

29. The formulation according to claim 1, wherein when the formulation is pulverized/milled and added to an alcoholic and/or non-alcoholic beverage, the rate of release of said at least one primary active substance in the loading dose is substantially the same or lower than the rate of release of said at least one primary active substance in the loading dose when the formulation is administered in an intact form.

30. The formulation according to claim 29, wherein the beverage is an alcoholic beverage.

31. The formulation according to claim 1, wherein the formulation comprises at least one excipient, wherein dissolution of the pulverized/milled formulation in alcoholic and/or non-alcoholic beverages causes the formulation to agglomerate.

32. The formulation according to claim 31, wherein the at least one excipient comprises at least one swellable material in such an amount that dissolution of the pulverized/milled formulation in alcoholic and/or non-alcoholic beverages causes the formulation to agglomerate.

33. The formulation according to claim 32, wherein said at least one swellable material is at least one pH independent polymer.

34. The formulation according to claim 32, wherein said at least one swellable material is selected from carbomers, polyethylene oxides or hydrophilic polymers that are lightly cross-linked, such cross-links being formed by covalent or ionic bonds, which interact with water and aqueous biological fluids and swell or expand to some equilibrium state.

35. The formulation according to claim 34, wherein said at least one swellable material comprises hydrophobic polymers.

36. The formulation according to claim 35, wherein the hydrophobic polymers are selected from Eudragit RL, Eudragit NE, Eudragit RS and/or Eudragit NM.

37. The formulation according to claim 36, wherein the at least one excipient comprises polyethylene oxide and Eudragit RL.

38. The formulation according to claim 7 further comprises at least one swellable material in such an amount that dissolution of the pulverized/milled formulation in alcoholic and/or non-alcoholic beverages causes the formulation to agglomerate, the amount of swellable material ranges from about 15 wt % to about 90 wt % of the maintenance dose and/or loading dose.

39. The formulation according to claim 1, wherein the formulation is objectionable to chewing, sucking, licking and/or holding in the mouth.

40. The formulation according to claim 38 further comprises a bittering agent and/or irritant.

41. The formulation according to claim 1, wherein the formulation is an oral formulation.

42. The formulation according to claim 1, wherein the formulation is a solid unit form.

43. The formulation according to claim 1, wherein the surface area covered by the Eudragit E in said at least one coat is greater than 5 mg/cm².

44. The formulation according to claim 42, wherein the surface area covered by the Eudragit E in the coat is greater than 10 mg/cm².

45. The formulation according to claim 42, wherein the surface area covered by the Eudragit E in the coat is greater than 20 mg/cm².

46. The formulation according to claim 42, wherein the surface area covered by the Eudragit E in the coat is from about 5 mg/cm² to about 100 mg/cm².

47. The formulation according to claim 42, wherein the surface area covered by the Eudragit E in the coat is from about 10 mg/cm² to about 100 mg/cm².

48. The formulation according to claim 42, wherein the surface area covered by the Eudragit E in the coat is from about 20 mg/cm² to about 100 mg/cm².

49. The formulation according to claim 1, wherein the formulation is capable of withstanding about a 350 N force.

50. The formulation according to claim 1, wherein the formulation is effective in preventing significant dose dumping in any beverage.

51. The formulation according to claim 1 further comprises at least one acid to facilitate release of any active substance in the formulation.

52. The formulation according to claim 7 further comprises at least one organic acid to facilitate release of any active substance in the formulation, wherein at least one of said at least one loading dose, said at least one maintenance dose, or said at least one coat comprises said at least one organic acid.

53. The formulation according to claim 52, wherein at least one of said at least one loading dose and said at least one coat comprises said at least one organic acid and the wt % ratio of the organic acid to said at least one primary active substance is from about 1:100 to about 100:1.

54. The formulation according to claim 52, wherein said at least one loading dose comprises from about 1 wt % to about 15 wt % by weight of said at least one organic acid based on the weight of the loading dose.

55. The formulation according to claim 52, wherein said at least one maintenance dose comprises from about 1 wt % to about 10 wt % by weight of said at least one organic acid based on the weight of the maintenance dose.

56. The formulation according to claim 52, wherein said at least one coat comprises from about 5 wt % to about 100 wt % by weight of said at least one organic acid based on the weight of said at least one coat.

57. The formulation according to claim 53 further comprises an overcoat, wherein the overcoat comprises said at least one organic acid and at least one polymer.

58. The formulation according to claim 57, wherein the amount of said at least one organic acid is from about 5 wt % to less than about 100 wt % of the overcoat.

59. The formulation according to claim 51, wherein said organic acid is selected from lactic acid, phosphoric acid, citric acid, malic acid, fumaric acid, stearic acid, tartaric acid, benzoic acid, or combinations thereof.

60. The formulation according to claim 1, wherein said at least one primary active substance is an addictive substance.

61. The formulation according to claim 60, wherein the addictive substance is an opiod agonist and/or a narcotic analgesic.
